(19) United States
(12) Patent Application Publication
(10) Pub. No.: US 2023/0417756 A1
(43) Pub. Date: Dec. 28, 2023

(54) SENSORS FOR UNBIASED PROTEOMIC STUDIES, METHOD OF MANUFACTURE AND USE THEREOF

(71) Applicant: NanoMosaic Inc., Woburn, MA (US)

(72) Inventors: John Boyce, Boston, MA (US); Audrey Warner, Boston, MA (US); Qimin Quan, Medford, MA (US); Joseph Wilkinson, Windham, NH (US)

(21) Appl. No.: 18/022,614

(22) PCT Filed: Aug. 26, 2021
(86) PCT No.: PCT/US2021/047830

§ 371 Date: Feb. 22, 2023

Related U.S. Application Data
(60) Provisional application No. 63/070,796, filed on Aug. 26, 2020.

(51) Int. Cl. G01N 33/68

(52) U.S. Cl. CPC G01N 33/6842; B82Y 15/00; G01N 2570/00

(57) ABSTRACT

The invention relates generally to articles (e.g., sensors) and methods that facilitate proteome, exome, or exome-codon sequence region wide interrogation for the discovery, screening and/or quantification of one or more proteins that contribute to a phenotype.

The present application claims the benefit of and priority to U.S. Patent Application No. 63/070,796, filed Aug. 26, 2020, the entire disclosure of which is incorporated herein by reference in its entirety.

### FIELD OF THE INVENTION

The invention relates generally to articles and methods relating to proteome, exome or exome-codon sequence (CDS) region wide interrogation for the discovery, screening and/or quantification of proteins that contribute to a phenotype.

### BACKGROUND

Several major challenges currently hinder the fields of medical diagnostics, biomarker discovery and drug discovery. Among the challenges is a lack of quantitative technologies that can be used to interrogate a wide dynamic range of proteins ranging from low abundance proteins, present, e.g., in a patient sample, to more abundant proteins.

The field of proteomic investigation typically involves the selection of proteins for interrogation based on a priori knowledge of pathways and biological interaction of molecules. The resulting protein panels are generally limited by number of proteins, as well as breadth of the proteins across the proteome, that can be interrogated. An additional challenge in determining proteins related to phenotype is providing an approach and a panel of proteins that facilitates a proteome-wide interrogation of wildtype proteins versus affected proteins in order to derive a bias-free approach across a wide range of proteins that may play a role in a condition under investigation.

Current protein panel selection is often limited by the readout technologies employed, where the number of unique sensors dedicated to each protein is not sufficient to cover a wide enough dynamic range to allow the breadth of biomarkers needed for proteome, exome or exome-CDS wide interrogation.

One commercially available approach uses a proximity extension assay where a pair of oligonucleotide-labeled antibodies (“probes”) are allowed to pair-wise bind to the target protein present in the sample in a homogeneous assay, with no need for washing. When the two probes are in close proximity, a novel PCR target sequence is formed by a proximity-dependent DNA polymerization event. The resulting target sequence is subsequently detected and quantified using standard real-time PCR (RT-PCR). A limitation of this approach is the restricted dynamic range and thus the number of proteins that can be interrogated.

Furthermore, scientific and medical investigators typically are limited to selecting targets that are disease-focused, which typically results in panels that facilitate interrogation of a limited number (e.g., under 500) proteins. For example, a commercially available cardiovascular panel enables a multiplex immunoassay (a proximity extension assay) for analysis of approximately 90 cardiovascular disease (CVD)-related protein biomarkers. In another example, a multiplex immunoassay inflammation panel can be interrogated via proximity ligation assay that facilitates the analysis of approximately 90 inflammation-related proteins.

In another approach, antibody-conjugated bead sets detect analytes in a multiplexed sandwich immunoassay format. Each bead in the set is identified by a unique content of two addressing dyes, with a third dye used to read out binding of the analyte via a biotin-conjugated antibody and streptavidin-conjugated second step detector. Data is acquired on a dedicated flow cytometry-based platform. However, such an approach has a limited dynamic range that restricts the markers that can be interrogated and therefore does not facilitate true proteome-wide interrogation. For example, exemplary assays contain a 50-plex bead kit that permit the analysis of 50 human cytokines and chemokines.

Despite the efforts that have been made to date, there is still a need for new approaches for interrogating a significant number of high and low abundance proteins across the proteome, exome or exome-CDS in a bias-free manner, which can be used to identify new biomarkers for a given phenotype.

### SUMMARY

The invention is based, in part, upon the development of an approach for interrogating a significant number of proteins (e.g., high and low abundance proteins) encoded across the genome in a bias-free manner. The approach can be used in conjunction with sensor and readout technologies that facilitate bias-free proteomic analyses.

In one aspect, the disclosure provides a method of determining a protein panel including a set of test proteins selected from a whole protein coding genome of a species to which a study subject belongs or is related to. The method comprises: (a) splicing protein coding genes (e.g., (i) both introns and exons, (ii) exons or (iii) coding-sequence regions) from a whole genome of a species of interest to construct a protein-coding genome (e.g., (i), proteome, (ii) exome, or (iii) exome-CDS, respectively); (b) determining a plurality of marker locations substantially evenly spaced across the protein-coding genome (e.g., (i), proteome, (ii) exome, or (iii) exome-CDS, respectively); and (c) identifying a protein associated with each marker location across the protein-coding genome (e.g., (i), proteome, (ii) exome, or (iii) exome-CDS, respectively) to produce the set of test proteins, wherein each protein is encoded by a gene that includes a single nucleotide polymorphism (SNP) located close to each marker location in the protein-coding genome.

In certain embodiments, the protein coding genes include both exons and introns, and the protein-coding genome is a proteome. Alternatively, the protein coding genes are exons, and the protein-coding genome is an exome. Alternatively, the protein coding genes are coding sequence (CDS) regions and the protein-coding genome is an exome-CDS.

It is contemplated that any of the foregoing methods may include one or more of the following features. For example, the SNPS may be synonymous SNPS, non-synonymous SNPS, or a combination thereof. The marker locations may be spaced apart from one another by about 25 kb, 50 kb, 100 kb, 200 kb, 300 kb, 600 kb, 1,200 kb, 6,000 kb, or 12,000 kb across the protein-coding genome, exome or exome-CDS.

The SNP may be the closest SNP to the marker location in the protein-coding genome, exome, or exome-CDS. The SNP is the closest non-synonymous SNP to the biomarker location, where a binding moiety can specifically bind a protein that is encoded by the gene containing the non-synonymous SNP. The SNP may be the closest synonymous SNP to the marker location, where a binding moiety can specifically bind a protein that is encoded by the gene containing the synonymous SNP.

In another aspect, the invention provides a method of determining a protein panel comprising a set of test proteins selected from a whole protein coding genome of a species to which a study subject belongs or is related to. The method comprises: (a) splicing protein coding genes (e.g., (i) both introns and exons, (ii) exons, or (iii) CDSs) from a whole genome of a species of interest to construct a protein-coding genome (e.g., (i) proteome, (ii) exome or (iii) exome-CDS, respectively), (b) determining a plurality of marker locations substantially evenly spaced across the protein-coding genome (e.g., (i) proteome, (ii) exome or (iii) exome-CDS, respectively); and (c) identifying a protein associated with each marker location across the protein-coding genome (e.g., (i) proteome, (ii) exome or (iii) exome-CDS, respectively) to produce the set of test proteins, wherein each protein is the protein encoded by a region of the genome in which the associated marker is located.

In certain embodiments, the protein coding genes include both exons and introns, and the protein-coding genome is a proteome. Alternatively, the protein coding genes are exons, and the protein-coding genome is an exome. Alternatively, the protein coding genes are coding sequence (CDS) regions and the protein-coding genome is an exome-CDS.

It is contemplated the marker locations may be spaced apart from one another by about 25 kb, 50 kb, 100 kb, 200 kb, 300 kb, 600 kb, 1,200 kb, 6,000 kb, or 12,000 kb across the protein-coding genome, exome, or exome-CDS.

In another aspect, the disclosure provides a sensor for detecting the presence, or quantifying the amount of a plurality of proteins in a sample harvested from a study subject thereby to conduct a bias-free proteome, exome or exome-CDS association study on the sample. The sensor comprises a plate defining a plurality of addressable wells, each well comprising a grid disposed therein, wherein (i) the grid comprises a plurality of nanostructure arrays with each nanostructure array comprising a plurality of nanostructures, and (ii) each nanostructure array is functionalized with one or more binding moieties for binding one or more proteins of a set of test proteins for conducting a bias-free proteome, exome or exome-CDS association study. Optionally, the set of test proteins is previously determined by: (a) determining a plurality of marker locations substantially evenly spaced across a protein-coding genome, exome, or exome-CDS of a species to which the study subject belongs or is related to; and (b) identifying a protein associated with each marker location across the protein-coding genome, exome, or exome-CDS to produce the set of test proteins, wherein each protein is encoded by a gene that includes a single nucleotide polymorphism (SNP) located close to each marker location in the exome.

It is contemplated that the sensor can be configured in a variety of different ways. For example, the SNPS may be synonymous SNPS, non-synonymous SNPS, or a combination thereof. The marker locations may be spaced apart from one another by about 25 kb, 50 kb, 100 kb, 200 kb, 300 kb, 600 kb, 1,200 kb, 6,000 kb, or 12,000 kb across the protein-coding genome, exome, or exome-CDS.

The sensor may include at least 20 different binding moieties for binding each member of the set of test proteins.

The SNP may be the closest SNP to the marker location in the protein-coding genome, exome, or exome-CDS. The SNP may be the closest non-synonymous SNP to the marker location, where a binding moiety can specifically bind a protein that is encoded by the gene containing the non-synonymous SNP. The SNP may be the closest synonymous SNP to the marker location, where a binding moiety can specifically bind a protein that is encoded by the gene containing the synonymous SNP.

The SNP may be located less than 1,000 bases from a corresponding marker location. All the SNPs may be located less than 1,000 bases from each corresponding marker location.

All the nanostructure arrays within a well may be functionalized with a binding moiety (e.g., an antibody, a nanobody, an aptamer, or an affinity probe) for binding a specific protein within the set of test proteins. A portion of the nanostructure arrays within a well may be functionalized with a binding moiety for binding a specific protein within the set of test proteins.

Each nanostructure may comprise or consist essentially of a nanoneedle. The nanostructures (e.g., nanoneedles) may be integral with at least one of a planar support or a flexible substrate.

In another aspect, the disclosure provides a method of producing a sensor for detecting the presence, or quantifying the amount, of a plurality of proteins in a sample harvested from a study subject thereby to conduct a bias-free proteome, exome or exome-CDS association study on the sample. The method comprises: (a) determining a plurality of marker locations substantially evenly spaced across an protein-coding genome, exome or exome-CDS of a species to which the study subject belongs or is related to; (b) identifying a protein associated with each marker location across the protein-coding genome, exome or exome-CDS to produce a set of test proteins, wherein each protein is encoded by a gene that includes a single nucleotide polymorphism (SNP) located closely to each marker location in the exome; and (c) functionalizing nanostructures of the sensor with a plurality of different binding moieties each capable of binding a protein in the set of test proteins thereby to detect the presence, or quantify the amount, of the test proteins if present in the sample.

It is contemplated that the method may include one or more of the following features. Steps (a)-(c) may be repeated to thereby produce a series of sensors, wherein the marker locations used to create a second sensor are shifted by a predetermined distance from the marker locations used to create a first sensor. The marker locations may be spaced apart from one another by 25 kb, 50 kb, 100 kb, 200 kb, 300 kb, 600 kb, 1,200 kb, 6,000 kb, or 12,000 kb across the protein-coding genome, exome, or exome-CDS.

The sensor may include at least 20 different binding moieties for binding the set of test proteins. The binding moiety may be an antibody, nanobody, aptamer or an affinity probe.

The SNPs may be synonymous SNPs, non-synonymous SNPs, or a combination thereof. Depending upon the circumstances, the SNP may be the closest SNP to the marker location in the protein-coding genome, exome, or exome-CDS. The SNP may be the closest non-synonymous SNP to the marker location, where a binding moiety can specifically bind a protein that is encoded by the gene containing the non-synonymous SNP. Alternatively, the SNP may be the closest synonymous SNP to the marker location, where a binding moiety can specifically bind a protein that is encoded by the gene containing the synonymous SNP. The SNP may be located less than 1,000 bases from a corresponding marker location. In certain embodiments, the SNPs may be located less than 1,000 bases from each corresponding marker location.

The disclosure also provides a sensor produced by any of the foregoing methods. The sensor may include a plurality of nanostructures functionalized with a plurality of different binding moieties each capable of binding a protein in the set of test proteins thereby to detect the presence, or quantify the amount, of the test proteins if present in the sample.

In another aspect, the disclosure provides a method of conducting a bias-free proteome, exome or exome-CDS-wide association study on a sample of interest. The method comprises (a) applying at least a portion of the sample to any of the sensors described herein; (b) detecting detectable signals from the nanostructures of the sensor; and (c) determining from the detectable signals the presence and/or amount of the test proteins in the sample.

It is contemplated that the method may include one or more of the following features. Steps (a)-(c) may be repeated with at least one additional sensor to screen a protein panel of the sample of interest. The step of detecting detectable signals may comprise detecting a change in a property (e.g., an optical property) of at least a portion of the nanostructures. The sample may be diluted or not diluted prior to application to the sensor. Depending upon the circumstances, the sample may be a body fluid, a tissue extract, or a cell supernatant.

Other advantages and novel features of the present disclosure will become apparent from the following detailed description of various non-limiting embodiments when considered in conjunction with the accompanying figures.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the disclosure. In the following description, various embodiments of the present invention are described with reference to the following drawings.

FIGS. 1A-1F are directed to methods of identifying markers and marker locations in a genome of interest and associated proteins, sensors and features of such sensors. FIG. 1A is a schematic diagram illustrating an approach for identifying markers evenly spaced at marker locations positioned across a genome of interest, in accordance with an embodiment of the invention. FIG. 1B is a schematic diagram illustrating the determination of a family or families of proteins represented by the selection of at least one member from the family within at least 100 base pairs of the marker location. FIG. 1C is a schematic diagram illustrating the selection of evenly spaced marker nucleotides across the exome and at least one cSNP in between a pair of marker nucleotides, which is at most 3 kb from the nucleotide marker, in accordance with an embodiment of the invention. FIG. 1D is a schematic diagram illustrating the selection of evenly spaced nucleotides across the exome and at least one nscSNP in between a pair of marker nucleotides, which is at most 10 kb from the nucleotide marker, in accordance with an embodiment of the invention. FIG. 1E is a schematic diagram illustrating a panel with a plurality of wells, each well containing a grid of nanostructure arrays, in accordance with an embodiment of the invention. FIG. 1F is a schematic illustration showing the dynamic range of a sensor in accordance with an embodiment of the invention in comparison to prior art assays.

FIG. 2A is a schematic representation of different formats of series of nanostructures in a sensor of interest. FIG. 2B is a schematic illustration depicting a series of exemplary sensors for measuring ultra-low, low, medium, and high concentrations of analytes.

FIGS. 3A-3C show the operability of exemplary sensors of the invention in measuring analyte over a large dynamic range. FIG. 3A is a schematic illustration depicting a sensor containing both digital and analog (color shifting) nanostructure arrays, in accordance with an embodiment of the invention. FIG. 3B is a pictorial representation depicting the quantification of Tau protein over a 6 log dynamic range by a combination of digital single molecule quantification (left hand panel) and by analog quantification (right hand panel). FIG. 3C is an image depicting the operability of a digital sensor as a function of analyte concentration.

FIG. 4 is a graph showing the digital and analog measurements of exemplary data generated by a sensor exemplified in FIG. 3B.

FIG. 5 is a pictorial representation of an exemplary silicon wafer-based sensor containing both a series of digital nanostructures (25,600) and three series of analog nanostructures (1,000 per series), in accordance with an embodiment of the invention.

FIG. 6 is a pictorial representation of another exemplary silicon wafer-based sensor comprising a plurality of series of digital nanostructures and three series of analog nanostructures, in accordance with an embodiment of the invention.

FIG. 7 is a schematic illustration depicting cross-sectional views of exemplary nanostructures, in accordance with embodiments of the invention.

FIG. 8 is a schematic illustration depicting cross-sectional views of exemplary nanostructures composed of two different materials, in accordance with embodiments of the invention.

FIGS. 9A-9D are a series of cross-sectional schematic diagrams illustrating the fabrication of a series of exemplary nanostructures by photoresist patterning, development and etching processes, in accordance with an embodiment of the invention.

FIGS. 10A-10G are a series of cross-sectional schematic diagrams illustrating the fabrication of a series of exemplary nanostructures by deposition of a layer on a substrate, spin coating a photoresist on the deposited layer, patterning and developing the resist, evaporating metal on the resist, removal of the resist in a solution, etching the substrate, and removing the photoresist, in accordance with an embodiment of the invention.

FIGS. 11A-11F are a series of cross-sectional schematic diagrams illustrating the fabrication of a series of exemplary nanostructures by coating two layers on a substrate, patterning the top layer resist, developing the resist, evaporating materials on the patterned resist, lift-off and spin additional low viscosity materials to achieve a particular surface condition, in accordance with an embodiment of the invention.

FIG. 12A-12F are a series of cross-sectional schematic diagrams illustrating the fabrication of a series of exemplary nanostructures by patterning photoresist on an oxide substrate, developing the resist, depositing silicon on the resist, lift-off, and growth of silicon to grow additional structures on the patterned substrate, in accordance with an embodiment of the invention.

FIGS. 13A-13D are a series of cross-sectional schematic diagrams illustrating the patterning of photoresist with a mold, in accordance with an embodiment of the invention.

FIG. 14A is a schematic illustration showing a silicon wafer with multiple series of nanostructures and FIG. 14B is a schematic illustration showing an enlarged image of a single series of nanostructures, in accordance with an embodiment of the invention. FIG. 14C is a schematic diagram of an embodiment of the present invention, wherein a single antibody label-free assay on nanostructure needles is used. Antibodies coupled to the nanostructure needles capture specific analytes in a test sample to produce a quantifiable signal. FIG. 14D is a schematic diagram of an embodiment of the present invention, wherein a single-antibody on nanostructure needles is used. Antibodies coupled to the nanostructure needles capture specific analytes in a test sample to produce a quantifiable signal, and the resultant signal is amplified. FIG. 14E is a schematic diagram of an embodiment of the present invention, wherein a dual antibody (sandwich) assay on nanostructure needles is used. The first antibody is coupled to the nanostructure needles to capture analytes in a test sample to produce a quantifiable signal, a second antibody is added to the reaction to form a sandwich, and the resultant signal is amplified.

FIGS. 15A-15D are schematic depictions of the gasket-based approach sensor design. FIG. 15A depicts a four-plex gasket. FIG. 15B depicts a hybrid 16-plex gasket covering half the sensor and a standard 96-well plate covering the other half FIG. 15C depicts a two gasket-layer approach, where a first layer comprises a four-plex gasket, and a second gasket is layered to cover four of the four-plex wells. FIG. 15D depicts a hybrid four-plex gasket with a second gasket layer covering four of the four-plex wells covering half the sensor and a standard 96-well plate covering the other half.

FIGS. 16A and 16B are perspective views of a nanosensor assembly (consumable) incorporating series of nanostructures in accordance with an embodiment of the invention.

FIGS. 17A and 17B are schematic representations of a cartridge assembly comprising a wafer substrate, gasket and retaining base (FIG. 17A) and an exploded perspective view showing the components of the cartridge assembly (FIG. 17B).

FIG. 18 is a schematic representation of a single plex cartridge and a 1,000-plex cartridge, in accordance with embodiments of the invention.

FIG. 19 is a perspective view of a detection system for use with a sensor, in accordance with an embodiment of the invention.

FIG. 20 is a schematic illustration depicting an exemplary optical detection system for imaging an exemplary sensor, in accordance with an embodiment of the invention.

FIG. 21 is a schematic illustration depicting the interrogation of a sensor, in accordance with an embodiment of the invention. The readout signal can be optical (e.g., imaging), electrical, or mechanical.

FIG. 22 is a schematic representation showing the data analysis of the output of an exemplary sensor containing digital nanostructures.

FIG. 23 is a flowchart illustrating an algorithm in accordance with an embodiment of the invention.

FIGS. 24A and 24B are schematic illustrations depicting series of nanostructures configured to detect and/or quantify multiple analytes at the same time, in accordance with an embodiment of the invention.

FIG. 25 is a schematic illustration depicting the interaction between an analyte and a nanostructure, in accordance with an embodiment of the invention.

FIG. 26 is a schematic representation depicting the binding capacity of a nanostructure, by capturing, from left to right, 1, 2 and 5 analytes, in accordance with an embodiment of the invention.

FIG. 27 is a schematic illustration depicting a non-saturating assay where there are fewer analytes than the number of nanostructures capable of capturing the analytes, in accordance with an embodiment of the invention.

FIG. 28 is a schematic illustration depicting series of nanostructures in an array under non-saturating assay conditions where analytes are bound by a fraction of the nanostructures in the array, in accordance with an embodiment of the invention.

FIG. 29 is a schematic representation depicting an exemplary label-free immunoassay.

FIG. 30 is a schematic representation depicting an exemplary label-based immunoassay.

FIG. 31 is a schematic illustration of an exemplary particle-based assay for determining the presence and/or amount of analyte (antigen) using a pair of antibodies (Ab1 and Ab2) that bind the antigen, where binding occurs in solution prior to detection via (Ab2) antibody capture by an activated nanostructure, in accordance with an embodiment of the invention.

FIG. 32 is a schematic illustration of an exemplary particle-based assay for determining the presence and/or amount of analyte (antigen) using a pair of antibodies (Ab1 and Ab2) that bind the antigen, wherein binding occurs in solution prior to detection via (Ab2) antibody capture by an activated nanostructure, in accordance with an embodiment of the invention.

FIG. 33 is a schematic illustration of an exemplary particle-based assay for determining the presence and/or amount of analyte (antigen) using a pair of antibodies (Ab1 and Ab2) that bind the antigen, wherein binding occurs in solution prior to detection via enzyme (HRP) capture by an activated nanostructure, in accordance with an embodiment of the invention.

FIG. 34 is a schematic illustration of an exemplary particle-based assay for determining the presence and/or amount of analyte (antigen) using a pair of antibodies (Ab1 and Ab2) that binds the antigen, wherein binding occurs in solution prior to detection via oligonucleotide capture by a nanostructure functionalized with a complimentary oligonucleotide, in accordance with an embodiment of the invention.

FIGS. 35A-35C are schematic illustrations depicting reagents for use in an exemplary multiplex assay.

FIG. 36A-H depicts standard titration curves across a concentration range from 1 pg/ml to 10,000 pg/ml for an array of cytokine antibodies tested in the gasket-based design, including IL-1b (FIG. 36A), IL-2 (FIG. 36B), IL-10 (FIG. 36C), IL-15 (FIG. 36D), IL-6 (FIG. 36E), IL-8 (FIG. 36F), GM-CSF (FIG. 36G), and IP-10 (FIG. 36H), respectively.

### DETAILED DESCRIPTION

The present disclosure is based, in part, upon the development of an approach for interrogating a significant number of proteins (e.g., high and low abundance proteins) encoded across the genome in a bias-free manner. The disclosure provides a method for implementing a bias-free proteome, exome or exome-CDS association study of a species (or related to a species) of a subject of interest. In particular, provided is a method for identifying proteins associated with corresponding nucleic acid markers spaced apart at marker locations disposed throughout a proteome, exome or exome-CDS of a species of interest, methods of making sensors for interrogating such proteins, sensors for performing an interrogation of proteins encoded in proteome, exome, or exome-CDS, and methods of using such sensors.

Embodiments of the present invention include protein panels, sensors, assays, and biochemical processes for detecting the presence and/or quantifying amounts of proteins involved in a specific phenotype. Embodiments of this invention may be used, for example, for diagnostic, biomarker discovery or drug development applications.

Described herein is the preparation of a panel of proteins selected from the entire proteome, exome or exome-CDS of a species, which includes selecting proteins (e.g., proteins corresponding to SNPs) in proximity to nucleotide markers evenly spaced throughout a certain region on the genome (e.g., protein-coding genome, exome or exome-CDS (coding sequences)) of the species. The human exome contains approximately thirty million bases and encodes the proteins that are present in the human proteome. The approaches described herein can be used to identify proteins for performing an unbiased interrogation of the entire proteome, exome or exome-CDS of a species of interest.

Described herein are sensors that include nanostructures, such as nanoneedles, functionalized with binding moieties corresponding to determined protein panels.

Also provided are biological assays that work in conjunction with nanostructures, and methodologies that utilize nanostructures that are approximately one thousand times smaller than beads, thereby allowing investigators to employ many more landing sites than there are target molecules, allowing for at least six orders of dynamic range. The wide dynamic range allows for construction of a proteome, exome or exome-CDS wide interrogation panel for bias-free analysis. A novel approach is provided for selecting proteins to construct a panel that covers the proteome to maximize coverage and drive bias-free results.

In some embodiments, the described methodology may be applied in any system with a ratio of at least 2:1 of the number of sensors (e.g., comprising nanostructures) to proteins under interrogation.

### Method of Generation of a Protein Panel

In an aspect, the disclosure provides a method of determining a protein panel including a set of test proteins selected from a whole protein coding genome of a species to which a study subject belongs or is related to. The method comprises: (a) splicing protein coding genes (e.g., (i) both introns and exons, (ii) exons or (iii) coding-sequence regions) from a whole genome of a species of interest to construct a protein-coding genome (e.g., (i), proteome, (ii) exome, or (iii) exome-CDS, respectively); (b) determining a plurality of marker locations substantially evenly spaced across the protein-coding genome (e.g., (i), proteome, (ii) exome, or (iii) exome-CDS, respectively); and (c) identifying a protein associated with each marker location across the protein-coding genome (e.g., (i), proteome, (ii) exome, or (iii) exome-CDS, respectively) to produce the set of test proteins, wherein each protein is encoded by a gene that includes a single nucleotide polymorphism (SNP) located close to each marker location in the protein-coding genome. In certain embodiments, the protein coding genes include both exons and introns, and the protein-coding genome is a proteome. Alternatively, the protein coding genes are exons, and the protein-coding genome is an exome. Alternatively, the protein coding genes are coding sequence (CDS) regions and the protein-coding genome is an exome-CDS.

As used herein, the term “splicing” refers to the process whereby a given subset of nucleotide sequences (e.g., protein-coding genes, exons, and coding-sequence regions) are selected from a given genome, and the resulting nucleotide sequence are then rejoined (e.g., in the same spatial relationship with respect to one another in the genome). In some embodiments, the nucleotide sequences are spliced together by selection of protein-coding genes (e.g., sequences that comprise exons and introns), and resulting protein-coding genes are rejoined to form a proteome. In some embodiments, the nucleotide sequences are spliced together by selection of exons (e.g., sequences that comprise coding-sequence regions and untranslated regions), and resulting exons are rejoined to form an exome. In some embodiments, the nucleotide sequences are spliced together by selection of coding-sequence regions (CDS) and the resulting CDSs are rejoined to form an exome-CDS.

As used herein, the terms “marker” or “marker nucleotide” or the like in the context of a protein-coding genome is understood to mean a nucleotide or group of nucleotides at a given marker location. As used herein, the term “marker location” is understood to mean the location of where markers or marker nucleotides are positioned within a protein-coding genome (e.g., a proteome, exome, or exome-CDS).

In some embodiments, a protein-coding gene refers to the nucleotide sequence associated with a protein and includes the exons and introns of such protein. In some embodiments, a “protein-coding genome” refers to the nucleotide sequences (e.g., exons and introns) of all proteins encoded by the genome, and may also be referred to as a proteome.

In some embodiments, a protein-coding gene refers to the nucleotide sequence associated with a protein and includes the exons (e.g., coding-sequence region (CDS) and untranslated regions (e.g., 5′ and 3′ UTRs)) of such protein. In this embodiment, the intron sequences are removed. In some embodiments, a “protein-coding genome” refers to the nucleotide sequences of all proteins and includes exons (e.g., coding-sequence region (CDS) and untranslated regions (e.g., 5′ and 3′ UTRs)) of all proteins encoded by the genome, and may also be referred to as an exome.

In some embodiments, a protein-coding gene refers to the nucleotide sequence associated with a protein and includes the coding-sequence regions (CDS) of such protein. In this embodiment, introns and untranslated regions of exons are removed. In some embodiments, a “protein-coding genome” refers to the nucleotide sequences of all proteins and includes CDSs of all proteins encoded by the genome, and may also be referred to as an exome-CDS.

In another aspect, the invention provides a method of determining a protein panel comprising a set of test proteins selected from a whole protein coding genome of a species to which a study subject belongs or is related to. The method comprises: (a) splicing protein coding genes (e.g., (i) both introns and exons, (ii) exons, or (iii) CDSs) from a whole genome of a species of interest to construct a protein-coding genome (e.g., (i) proteome, (ii) exome or (iii) exome-CDS, respectively), (b) determining a plurality of marker locations substantially evenly spaced across the protein-coding genome (e.g., (i) proteome, (ii) exome or (iii) exome-CDS, respectively); and (c) identifying a protein associated with each marker location across the protein-coding genome (e.g., (i) proteome, (ii) exome or (iii) exome-CDS, respectively) to produce the set of test proteins, wherein each protein is the protein encoded by a region of the proteome in which the associated marker is located. In certain embodiments, the protein coding genes include both exons and introns, and the protein-coding genome is a proteome. Alternatively, the protein coding genes are exons, and the protein-coding genome is an exome. Alternatively, the protein coding genes are coding sequence (CDS) regions, and the protein-coding genome is an exome-CDS.

As described herein a protein panel is generated by selection of proteins from the entire proteome, exome or exome-CDS of a species, with the proteins corresponding to SNPs in proximity to nucleotide markers evenly spaced throughout a certain region on the genome (e.g., protein-coding genome, exome or exome-coding sequence (CDS)) of the species. In certain embodiments, the protein panel is generated by selection of proteins from the entire proteome of a species, with the proteins selected based upon proximity to nucleotide markers evenly spaced throughout a certain region on the genome (e.g., protein-coding genome, exome or exome-coding sequence (CDS)) of the species, i.e., independent of SNPs.

Following construction of a protein-coding genome, exome or exome-CDS, a plurality of marker locations substantially evenly spaced across the protein-coding genome, exome or exome-CDS are noted, and a protein associated with each marker location across the protein-coding genome, exome or exome-CDS is selected to produce a protein panel. In certain embodiments, each protein is encoded by a gene that includes a single nucleotide polymorphism (SNP) located close to each marker location. The marker locations may be spaced apart from one another by a selected distance, such as 25 kb, 50 kb, 100 kb, 200 kb, 300 kb, 600 kb, 1,200 kb, 6,000 kb, or 12,000 kb across the exome. Depending upon the circumstances, the closest single nucleotide polymorphism (SNP) to each nucleotide marker is then identified. In some embodiments, one or all of the SNPs may be located less than 1,000 bases from a corresponding nucleotide marker location.

The protein associated with the SNP (i.e., the protein encoded by a gene that includes the SNP) is then identified, in order to produce a protein panel. The SNPs may be synonymous SNPs, non-synonymous SNPs, or a combination thereof. The SNP may be the closest SNP to the marker location in the exome. In some embodiments, the SNP may be the closest non-synonymous SNP to the marker location, where a binding moiety can specifically bind a protein that is encoded by the gene containing the non-synonymous SNP. In other embodiments, the SNP is the closest synonymous SNP to the marker location, where a binding moiety can specifically bind a protein that is encoded by the gene containing the synonymous SNP. In some embodiments, the binding moiety is an antibody, nanobody, affinity probe, or an aptamer.

In some embodiments, the selected protein has a commercially available antibody. In some embodiments, the selected protein does not have a commercially available antibody, and a new antibody is generated using techniques known in the art. In some embodiments, the selected protein does not have a commercially available antibody, and, for example, the second-closest SNP to the nucleotide marker is selected, and the protein including said second-closest SNP is included in the sensor.

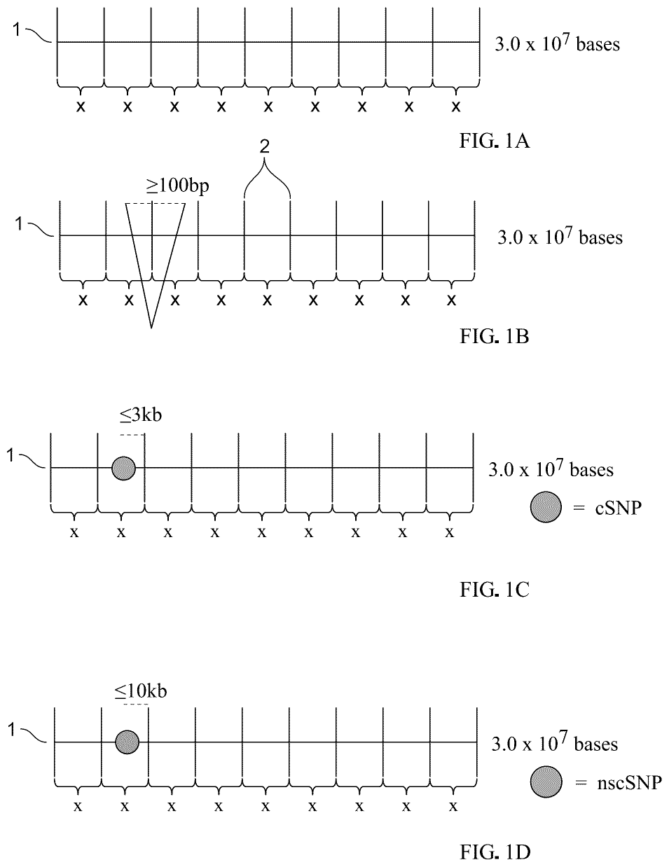

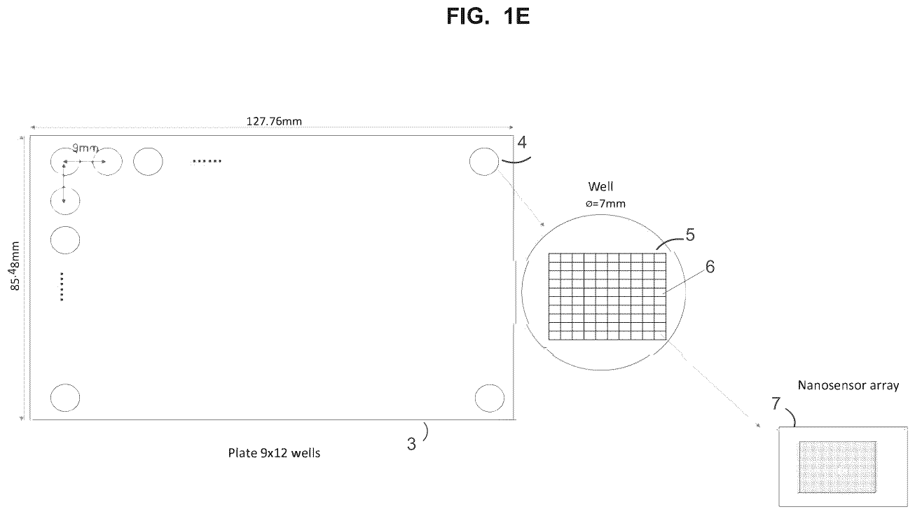

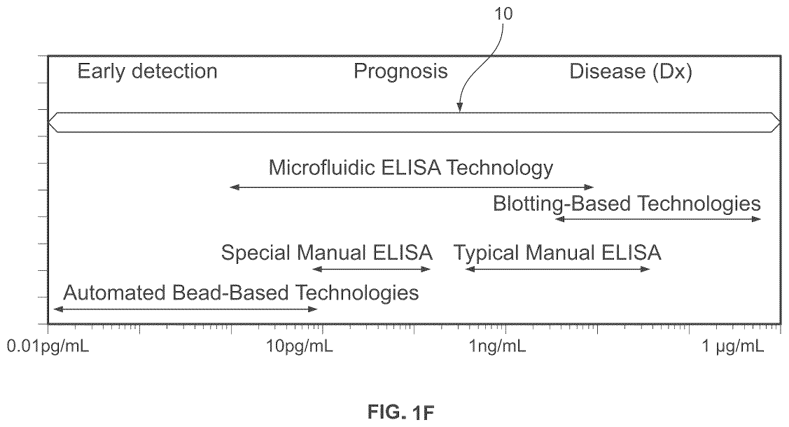

Examples of these methodologies are described with respect to FIGS. 1A-1D as follows. Referring to FIG. 1A, in accordance with embodiments of the invention, a sequence 1 (e.g., a protein-coding genome, exome or exome-CDS) is assembled and aligned. In some embodiments, a protein-coding genome is constructed by splicing all the protein coding genes (e.g., nucleotide sequences comprising both exons and introns) across the whole genome into a continuous sequence. In some embodiments, an exome is constructed by splicing all the exons (e.g., nucleic acid sequences comprising both untranslated sequences (e.g., 5′ and the 3′ UTRs) and coding sequences) across the whole genome. In some embodiments, an exome-CDS is constructed by splicing only the coding sequence regions (e.g., exons with the untranslated regions (e.g., the 5′ and the 3′ UTRs) removed) across the whole genome. The assembly and alignment of the protein-coding genome, exome or exome-CDS take place prior to the steps outlined with reference to FIGS. 1B-1D.

Referring to FIG. 1B, in one embodiment, a panel is constructed by choosing evenly spaced nucleotides (i.e., markers 2) across the exome, as well as 100 base pairs on either side of that marker. Thus, in this example, a distance X between two adjacent markers 5 is 200 base pairs. This area of 100 base pairs is considered a “region” of the exome to select a protein. At least 0.10%, 10% or 10% of the exome is selected. One protein from the family of proteins that each region codes for is chosen for inclusion on the protein panel.

Referring to FIG. 1C, in another embodiment, focused on coding single nucleotide polymorphisms (cSNPs), evenly spaced markers X are chosen across the exome, and at least one cSNP is identified in between adjacent pairs of markers, within at most 3 kilobases (KB) distance from the marker. Each of these areas of 3 KB is considered a region. At least 0.1%, 1% or 10% of the exome is selected. One protein from the family of proteins that each region codes for is chosen for inclusion on the protein panel.

Referring to FIG. 1D, in another embodiment focused on non-coding single nucleotide polymorphisms (nscSNPs), evenly spaced markers are chosen across the exome, and at least one nscSNP is identified between adjacent pairs of markers, within at most 10 kilobases distance from the marker. Each of these areas of ±10 kilobases is considered a region. At least 0.1%, 1% or 10% of the exome is selected. One protein from the family of proteins that each region codes for is chosen for inclusion on the protein panel.

### Method of Manufacture of a Sensor

In another aspect, the disclosure provides a sensor for detecting the presence, or quantifying the amount of a plurality of proteins in a sample harvested from a study subject thereby to conduct a bias-free proteome, exome or exome-CDS association study on the sample. The sensor comprises a plate defining a plurality of addressable wells, each well comprising a grid disposed therein, wherein (i) the grid comprises a plurality of nanostructure arrays with each nanostructure array comprising a plurality of nanostructures, and (ii) each nanostructure array is functionalized with one or more binding moieties for binding one or more proteins of a set of test proteins for conducting an bias-free proteome, exome or exome-CDS wide association study. The terms “bias-free” or “unbiased” in the context of a proteome, exome or exome-CDS wide association study are used interchangeably and are understood to mean that target proteins (or biomarker proteins) for interrogation are selected based primarily on locations of the genes encoding the proteins or peptides in the genome of a species of interest, without consideration of whether the protein or peptide is associated with a specific disease, disorder, or biological pathway. Optionally, the set of test proteins is previously determined by: (a) determining a plurality of marker locations substantially evenly spaced across a protein-coding genome, exome, or exome-CDS of a species to which the study subject belongs or is related to; and (b) identifying a protein associated with each marker location across the protein-coding genome, exome, or exome-CDS to produce the set of test proteins, wherein each protein is encoded by a gene that includes a single nucleotide polymorphism (SNP) located close to each marker location in the exome.

The sensor enables detecting the presence or quantifying the amount of a plurality of proteins (e.g., a plurality of proteins from a protein panel generated as described above) in a sample harvested from a study subject, to conduct a bias-free proteome, exome or exome-CDS association study on the sample. A plurality of nucleotide marker locations substantially evenly spaced across a protein-coding genome, exome or exome-CDS of a given species are determined using the approaches described above. The marker locations may be spaced apart from one another by a selected distance, such as 25 kb, 50 kb, 100 kb, 200 kb, 300 kb, 600 kb, 1,200 kb, 6,000 kb, or 12,000 kb across the exome. In one embodiment, 100 random markers are selected from across the exome, with markers spaced 300 kb apart.

In certain embodiments, the closest single nucleotide polymorphism (SNP) to each nucleotide marker is then identified. In some embodiments, a nucleotide marker is equidistant to two or more SNPs, and the SNP is randomly selected. The protein associated with the SNP (i.e., the protein being encoded by a gene that includes the SNP) is then identified, to produce a set of randomly selected test proteins spanning the entire protein-coding genome, exome or exome-CDS. The SNPs may be synonymous SNPs, non-synonymous SNPs, or a combination thereof. The SNP may be the closest SNP to the marker location in the exome. In some embodiments, the SNP may be the closest non-synonymous SNP to the marker location, where a binding moiety can specifically bind a protein that is encoded by the gene containing the non-synonymous SNP. In other embodiments, the SNP is the closest synonymous SNP to the marker location, where a binding moiety can specifically bind a protein that is encoded by the gene containing the synonymous SNP.

In some embodiments, proteins are chosen independent of neighboring SNPs, i.e., based on their distance to the nucleotide marker. In some embodiments, a protein that is directly closest to the nucleotide marker is selected. In some embodiments, a protein that is closest to the nucleotide marker for which an antibody is commercially-available is selected.

In some embodiments, the selected protein has a commercially-available antibody or aptamer. In some embodiments, the selected protein does not have a commercially-available antibody or aptamer, and a new antibody is generated. In some embodiments, the selected protein does not have a commercially-available antibody or aptamer, and, for example, the second-closest SNP to the nucleotide marker is selected, and the protein including said second-closest SNP is included in the sensor. In some embodiments, no commercial antibodies or aptamers are available to the proteins that includes the third-closest SNP, recombinant antibodies or aptamers will be developed for the selected protein. For example, recombinant antibodies or nanobodies can be developed by screening libraries on a phase display or yeast display.

One or all of the SNPs may be located less than 1,000 bases from a corresponding nucleotide marker location.

Nanostructures of the sensor are functionalized with a plurality of different binding moieties each capable of binding a protein in the set of test proteins thereby to detect the presence, or quantify the amount, of the test proteins if present in the sample. The sensor may include a wide range of different binding moieties, such as at least 20, 25, 50, 100, 150, 300, 600, or 1200 different binding moieties, for binding the set of test proteins. The binding moiety may be an antibody, nanobody, affinity probe, or an aptamer.

In some embodiments, the binding moiety, e.g., antibody, is used to screen for the presence or absence of a protein. In some embodiments, the binding moiety, e.g., antibody, is used to screen for the total amount of a protein. In some embodiments, the binding moiety, e.g., antibody, is used to screen for particular variants of a protein, e.g., a mutant variant of the protein. In some embodiments, the binding moiety, e.g., antibody, is used to screen for particular post-translational modification of a protein, e.g., a phosphorylated or glycosylated form of the protein.

These steps may be repeated to produce a series of sensors, with the nucleotide marker locations used to create a second sensor being shifted by a predetermined distance from the marker locations used to create a first sensor. This approach can be repeated to create a series of sensors, wherein each sensor is capable of detecting proteins encoded by nucleotide sequences long the genome that are off-set from proteins that are detected by the other sensors in the series. Such iterative sensor production may be used to generate a series of unbiasedly selected marker proteins across the human proteome, exome or exome-CDS.

In some embodiments, following the initial screening of the unbiasedly selected proteins, there is a significant change in a protein of the sensor. A second, targeted-protein sensor (e.g., a sensor capable of detecting related proteins, such as family members), may be used to further probe changes in protein levels, protein signaling, etc.

Referring to FIG. 1E, a sensor for detecting presence or quantifying the amount of a plurality of proteins in a sample includes a plate. The plate 3 (also referred to herein as a panel or a protein panel) in accordance with an embodiment of the invention may include an array of addressable wells, e.g., 8×12 (96 plate), 16×24 (384 plate), 32×48 (1536 plate) wells. As an example, each well 4 of the 96 well plate includes a grid 5 disposed therein, e.g., a 10×10 grid, with each block 6 of the grid being, e.g., about 400 microns×400 microns, and functionalized with different binding moieties, e.g., antibodies. More specifically, each block 6 of the grid 5 includes one nanostructure array 7, with each nanostructure array including a plurality of nanostructures, as discussed below. Each nanostructure array is functionalized with one or more binding moieties, such as antibodies, nanobodies, affinity probes, or aptamers, for binding one or more proteins of a set of test proteins for conducting a proteome, exome or exome-CDS association study. In some embodiments, all the nanostructure arrays within a well are functionalized with a binding moiety for binding a specific protein within the set of test proteins. In other embodiments, a portion of the nanostructure arrays within a well are functionalized with a binding moiety for binding a specific protein within the set of test proteins.

The sensor may include about 25, 50, 100, 150, 300, 600, or 1200 different binding moieties for binding each member of the set of test proteins.

The set of test proteins is determined by first determining a plurality of marker locations substantially evenly spaced across the protein-coding genome, exome or exome-CDS of a species to which the study subject belongs or is related to. The marker locations may be spaced apart from one another by about 25 kb, 50 kb, 100 kb, 200 kb, 300 kb, 600 kb, 1,200 kb, 6,000 kb, or 12,000 kb across the protein-coding genome, exome or exome-CDS.

Then, a protein associated with each marker location across the protein-coding genome, exome or exome-CDS is identified to produce the set of test proteins. Each protein is encoded by a gene that includes a single nucleotide polymorphism (SNP) located close to each marker location in the exome. The SNPs may be synonymous SNPs, non-synonymous SNPs, or a combination thereof. The SNP may be the closest SNP to the marker location in the protein-coding genome, exome or exome-CDS. In some embodiments, the SNP is the closest non-synonymous SNP to the marker location, where a binding moiety can specifically bind a protein that is encoded by the gene containing the non-synonymous SNP. In other embodiments, the SNP is the closest synonymous SNP to the marker location, where a binding moiety can specifically bind a protein that is encoded by the gene containing the synonymous SNP. The SNP—or all the SNPs—may be located less than 1,000 bases from a corresponding marker location.

### Methods of Use

A bias-free proteome, exome or exome-CDS wide association study may be conducted on a sample of interest as follows. The sample may be, e.g., a body fluid (e.g., blood, serum, plasma, saliva, etc.), a tissue extract, or a cell supernatant. A portion of the sample may be applied to any embodiment of the sensor described above. Depending upon the circumstances, the sample may be or need not be diluted before application to the sensor.

Detectable signals from the nanostructures of the sensor are then quantified. For example, a change in property, e.g., an optical property, e.g., fluorescence, of at least a portion of the nanostructures may be detected. The presence and/or amount of the test proteins in the sample is determined from the detectable signals. These steps may be repeated with at least one additional sensor to screen the proteome, exome or exome-CDS of the sample of interest.

As used herein, the term “subject” refers to an organism to be tested by the methods and compositions of the present invention. Such organisms preferably include mammals (e.g., human, mouse, rat, guinea pig, dog, cat, horse, cow, pig, or non-human primate, such as a monkey, chimpanzee, baboon, and rhesus), and more preferably humans.

Applications of the sensors described in the present application include, without limitation, biomarker identification, diagnostics (e.g., diagnostics for identifying a subject with a disease or disorder, or companion diagnostics), patient stratification protocols, and drug-development. Biomarker identification applications include, without limitation, identification of biomarkers for a given phenotype of interest (e.g., tolerance to a drug or therapeutic, resistance to a drug or therapeutic, metabolic sensitivities, etc.) or for a particular disease-state (e.g., cardiovascular disease, inflammatory disease, autoimmune disease, psychological conditions, neurodegenerative disease, cancer, etc.). Such biomarkers may be associated with the presence of the phenotype and/or disease-state in a subject, or indicate an elevated risk of developing the phenotype and/or disease-state of the subject relative to the general population. Diagnostics applications include, without limitation, risk-assessment and/or identification of a particular disease-state in a subject (e.g., cardiovascular disease, inflammatory disease, autoimmune disease, psychological conditions, neurodegenerative disease, cancer, etc.) in an affected subject, companion diagnostics for identifying whether a subject may be responsive or non-responsive to a drug. Patient stratification applications include, without limitation, the identification of patients for clinical studies or identifying patients likely to respond to a given drug. Drug-development applications include, without limitation, screening of known or novel therapeutics and/or biologics for a particular disease-state (e.g., cardiovascular disease, inflammatory disease, autoimmune disease, psychological conditions, neurodegenerative disease, cancer, etc.) across the protein panel, for a desired response.

In BMC Res Notes (2019) 12:315, Piovesan et. al. extracted the information of human protein coding genes from the NCBI Gene Web. In one embodiment, based on Piovesan's Gene Table, the Gene ID, Gene symbol, Chromosome accession number, the start and end location of all protein-coding genes and displayed in the order of their location in the human genome from chromosome 1 to chromosome X and Y. All the protein coding genes are then spliced together for continuous numbering of the protein-coding genome, for a total length of 1,255,970,826 bp.

In one embodiment, to construct a 100-plex protein panel in a bias-free manner, 100 nucleotide position markers are placed along the spliced genes, each located at 12,559,708*i, where i is the sequence of the marker. The spacing between the markers is 12,559,708. For the i^{th} marker, using a Single Nucleotide Polymorphism Database (dbSNP), a SNP that is nearest to the position marker i is located. Then, the gene that contains the identified SNP is located and included in the panel as the i^{th} protein. The protein list following the above procedure is compiled and further described in Example 1 below.

In one embodiment, to construct a 100-plex protein panel in a bias-free manner, a protein panel is constructed from an exome (e.g., nucleotide sequences that exclude introns from the protein coding genes). One isoform of a protein can be was chosen from Piovesan's Gene Table (described above), and the start end locations of the 3′ UTR, CDS and 5′ UTR are recorded to identify exons. All exons can then be spliced together, which results in a total exome length of 62,184,186 bp.

A 100-plex protein panel can be generated in a bias-free manner from the above-described exome, by placing 100 position markers along the spliced genes, each located at 621,842*i, where i is the sequence of the marker. The spacing between the markers is 621,842 bp. For the i^{th} marker, using the Single Nucleotide Polymorphism Database (dbSNP), a SNP that was nearest to the position marker i can be located. Then, the gene containing the identified SNP is located and included in the panel as the i^{th} protein. The resultant protein list generated from the above protocol is shown in Table 5.

After the protein is identified, the detectable moiety (e.g., antibody, nanobody, affinity probe, or aptamer) specific to the protein will be incorporated on the surface of the sensor. In some embodiments, a recombinant antibody or nanobody can be developed with various display technologies (e.g., phase-display or yeast-display). In some embodiments, an aptamer can be developed with the SELECT technology. In one example, a single antibody or a dual antibody pairs can be developed for each of the targets. In one example, a dual antibody pair can be developed for each of the targets. Next, for a panel of 100 proteins, the 100 different affinity probes specific to each protein will be spotted on each grid with printing techniques such as inkjet or piezoelectric printing. The concentrations of the proteins can be measured, for example, using the methods described below.

Additional details regarding the sensor structure, operation, and fabrication as well as the functionalization of the nanostructures and assays, are provided below.

### I. Sensor Considerations

The sensors disclosed herein facilitate the detection and/or quantification, with high sensitivity over a large dynamic range, of the amount of an protein or peptide in a sample of interest. Also disclosed herein is a cartridge incorporating such a sensor, a detection system, and methods of using such a sensor, cartridge and system, to detect and/or quantify the amount of proteins or peptides in a sample in order to facilitate a proteome, exome or exome-CDS association study.

FIG. 1F illustrates the dynamic range 10 achievable with a sensor described herein that can detect analytes in a sample within a concentration range between less than 0.01 pg/mL (10 fg/mL) and 1 μg/mL or greater (at least 8 logs). In general, other commercially available assay systems (for example, typical manual ELISA, special manual ELISA, microfluidic-based ELISA assays, blotting-based technologies (e.g., Western blotting and dot blotting technologies) and automated bead-based technologies) can measure analytes in samples of interest but cannot measure analytes over the entire dynamic range achievable with a sensor disclosed herein. As a result, use of the sensor described herein may facilitate the measurement of concentrations of analyte over a concentration range that heretofore could only be achieved using a combination of prior art assay systems.

(A) Sensor Configurations

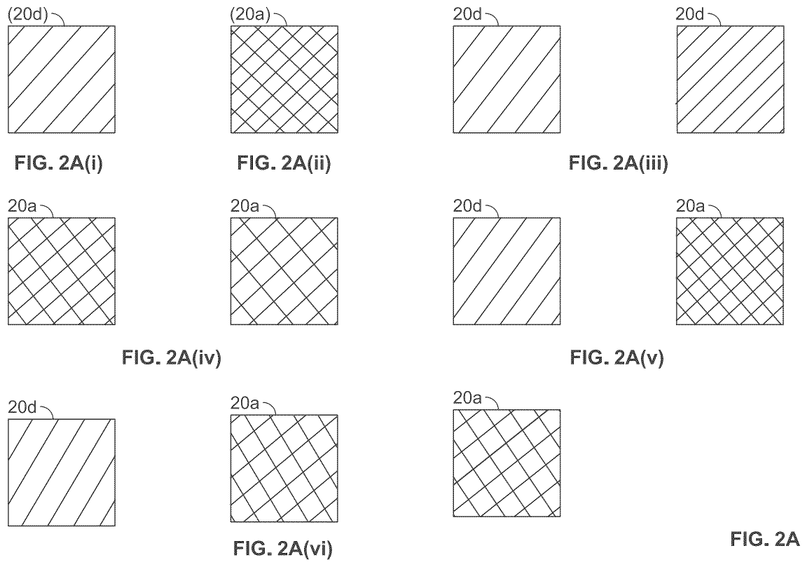

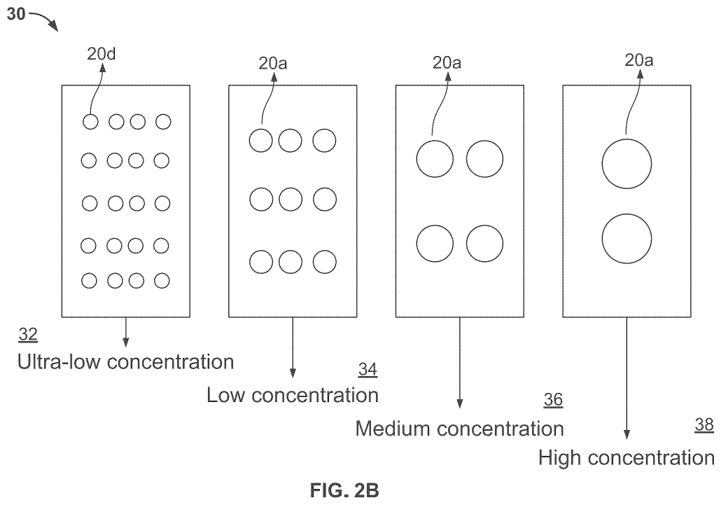

It is contemplated that the sensor may comprise nanostructures in a variety of configurations. For example, as shown in FIG. 2A, the sensor may comprise a first series of nanostructures 20d, for example, a series of nanostructures configured for digital quantification (FIG. 2A(i)); a second series of nanostructures 20a, for example, a series of nanostructures configured for analog quantification (FIG. 2A(ii)); two series of nanostructures 20d (FIG. 2A(iii)); two series of nanostructures 20a (FIG. 2A(iv)); two series of nanostructures one of 20d and one of 20a (FIG. 2A(v)); and three series of nanostructures one of 20d and two of 20a (FIG. 2A(vi)). It is contemplated that the sensor may comprise other series of nanostructures in different configurations depending upon the analytes (e.g., proteins or peptides) to be detected and the dynamic range desired.

As used herein, the term “nanostructure” is understood to mean any structure, for example, a nanosensor, that has at least one dimension having a length in the range of at least 1 nm to less than 1,000 nm. As used herein, the term “digital quantification” is understood to mean a quantification process whereby individual nanostructures in a series of nanostructures are detected (for example, optically detected) that flip from one state to another upon binding one or more analytes. A “digital series” or “digital array” is understood to mean a respective series or array of nanostructures configured to permit digital quantification.

As used herein, the term “analog quantification” is understood to mean a quantification process whereby a substantially uniform change in a detectable property (for example, an optically detectable property, e.g., a color) of nanostructures in a series of nanostructures is detected, when the nanostructures bind a plurality of analytes. In certain embodiments, changes in the detectable property (e.g., color changes) occur as a function of the concentration of analyte in a sample of interest across a precalibrated concentration range of the analyte to be detected. The term “substantially uniform” is understood to mean that, at least 60%, 70%, 80%, 90% or 95% of the nanostructures share the same detectable property, for example, color. An “analog series” or “analog array” is understood to mean a respective series or array of nanostructures configured to permit analog detection.

In one exemplary sensor for detecting the presence, or quantifying the amount, of an analyte in a sample of interest, the sensor comprises a first region and a second region. The first region comprises a first series of nanostructures capable of binding the analyte and producing a detectable signal indicative of a concentration of the analyte in the sample within a first concentration range. The second region comprises a second series of different nanostructures capable of binding the analyte and producing a detectable signal indicative of a concentration of the analyte in the sample within a second, different concentration range, wherein the sensor is capable of quantifying the amount of analyte in a sample across both the first concentration range and the second concentration range. The first concentration range can have a lower detectable value than that of the second concentration range and/or the second concentration range can have a higher detectable value than that of the first concentration range. It is contemplated that the first concentration range can overlap the second concentration range.

It is understood that the sensors described herein are capable of detecting the concentration of analyte in the sample across a range (also referred to as dynamic range) spanning at least 3, 4, 5, 6, 7, 8, 9, 10, 11 or 12 orders of magnitude (or 3, 4, 5, 6, 7, 8, 9, 10, 11 or 12 logs). In certain embodiments, the sensor is capable of detecting the concentration of analyte in the sample across a concentration range spanning at least 5, 6, 7, 8 or 9 orders of magnitude (or 5, 6, 7, 8 or 9 logs). The sensor maybe configured to measure the concentration of a given analyte in the range from less than 1 pg/mL to greater than 100 ng/mL, from less than 0.1 pg/mL to greater than 1 μg/mL, or from less than 0.01 pg/mL to greater than 100 μg/mL, or from less than 1 fg/mL to greater than 1 mg/mL, where, for example, the sample does not need to be diluted prior to application to the sensor.

In one exemplary sensor, the first region comprises a first series of nanostructures capable of binding the analyte and producing a detectable signal indicative of a concentration of the analyte in the sample within a first concentration range, wherein individual nanostructures of the first series that bind the analyte are detected (for example, optically detected) upon binding the analyte, whereupon the concentration of analyte in the sample, if within the first concentration range, is determined from a number of individual nanostructures in the first series that have bound molecules of analyte. The second region comprises a second series of different nanostructures capable of binding the analyte and producing a detectable signal indicative of a concentration of the analyte in the sample within a second, different concentration range, wherein the concentration of analyte in the sample, if within the second concentration range, is determined by analog detection of a substantially uniform change in a detectable property (for example, an optically detectable property, such as color) of the nanostructures in the second region as a function of the concentration of the analyte, wherein the sensor is capable of quantifying the amount of analyte in a sample across both the first concentration range and the second concentration range.

The first concentration range has a lower detectable value than that of the second concentration range and/or the second concentration range has a higher detectable value than that of the first concentration range. It is contemplated that the first concentration range can overlap the second concentration range.

In each of the foregoing sensors, the first region of the sensor optionally comprises one or more of: (i) center-to-center spacing of adjacent nanostructures of at least 1 μm; (ii) a minimum cross-sectional dimension or diameter of each nanostructure of at least 10 nm; (iii) a maximum cross-sectional dimension or diameter of each nanostructure of no more than 200 nm; or (iv) a height of each nanostructure in a range of 50 nm to 1000 nm. The sensor optionally further comprises one or more of a (i) a fiducial marker or (ii) a nanostructure fabrication control feature.

It is contemplated that any of the sensors may comprises one or more of the following features. For example, it is contemplated that the sensor may further comprise a third region comprising a third series of further different nanostructures capable of binding the analyte and producing a detectable signal indicative of the concentration of the analyte in the sample within a third concentration range, wherein the sensor is capable of quantifying the amount of the analyte in the sample across the first, second and/or third concentration ranges.

Similarly, the nanostructures in any second series can comprise one of more of (i) an average height, (ii) an average volume, (iii) an average surface area, (iv) an average mass, and (v) an average number of analyte binding sites, that is greater than that of the nanostructures in the first series.

Furthermore, whenever the sensor comprises a third series, the nanostructures of the third series can comprise one of more of (i) an average height, (ii) an average volume, (iii) an average surface area, (iv) an average mass, and (v) an average number of analyte binding sites, that is greater than that of the nanostructures in any second series.

The nanostructures in the first series, and where applicable, the second and third series, are functionalized with a binding agent that binds the analyte, for example, binding agent, for example, a biological binding agent, that binds the analyte. The biological binding agent can be, for example, an antibody, an aptamer, a member of a ligand-receptor pair, an enzyme, or a nucleic acid. Under certain circumstances, it may be advantageous to use a binding agent in the first series that has a higher binding affinity for the analyte than the binding agent in a second, third or subsequent series.

The sensor may be designed to detect and/or quantify any analyte of interest in a sample. For example, the analyte may be a biological molecule, for example, a protein, including, for example, a protein, glycoprotein, lipoprotein, nucleoprotein and a peptide, including a peptide fragment of the foregoing proteins. Furthermore, a nanostructure or series of nanostructures in a given sensor may be configured to bind, detect and/or quantify a plurality of different analytes simultaneously or sequentially. For example, the sensor can comprise a plurality of different binding agents for detecting a corresponding plurality of different analytes in the test sample.

The sensor can be configured to detect the binding of an analyte via a change in an optical property, electrical property, or mechanical property. For example, sensor can be configured to detect the binding of an analyte via a change in an optically detectable property (for example, color, light scattering, refraction, or resonance (for example, surface plasmon resonance, electric resonance, electromagnetic resonance, and magnetic resonance)) of at least one series of nanostructures.

It is contemplated that the sensors may be configured in a variety of different ways. For example, at least one of the first, second or third series of nanostructures can comprise an array of nanostructures. Alternatively, each of the first, second and third series of nanostructures can comprise an array of nanostructures. It is contemplated that sensor may comprise a single series of nanostructures or a plurality of series of nanostructures, for example, a plurality of series of nanostructures operative to detect analyte within different concentration ranges. When the sensor comprises a plurality of series of nanostructures, the different series of nanostructures may operate (i) in the same manner (for example, via digital detection where single nanostructures are detected or quantified, or via analog detection where a cumulative change in an optical property of the nanostructures within a given series is detected as a function of concentration) or (ii) in a different manner, for example by a combination of digital detection and analog detection. Furthermore, it is contemplated that the sensor may comprise a plurality of different series that operate by digital detection and/or analog detection. For example, the sensor may comprise a plurality of series that operate to detect an analyte by digital detection within the same concentration range and/or a plurality of series that operate to detect an analyte by analog detection over different concentration ranges.

For example, during digital detection, in the first series of nanostructures, individual nanostructures that bind the analyte are detected upon binding either a single molecule of analyte or less than a predetermined number of molecules of the analyte, whereupon the concentration of analyte in the sample, if present in the first concentration range, is determined from a number of individual nanostructures in the first series that have bound molecules of the analyte. For example, the concentration of analyte in the sample is determined by digital counting of the number of individual nanostructures in the first series that have bound the analyte relative to either (i) a remaining number of individual nanostructures that have not bound analyte or (ii) a total number of nanostructures in the first series.

In this approach, a large number of nanostructures typically are densely patterned in a region of a sensor. When the number of the nanostructures is greater than the number of analytes to be detected, each nanostructure typically captures at most a single analyte, for example, based on mass transfer and Poisson distribution effects. Each nanostructure can have one of two states (for example, denoted as 1 or 0) depending upon whether analyte is bound or not. Accordingly, the number of nanostructures with state 1 after exposure to a sample with analytes can equal to the number of analytes. In certain embodiments, each individual nanostructure may have only a limited number of binding sites to capture one or a few (for example, less than 10) analytes, e.g., proteins or peptides. Each nanostructure has a corresponding signal scale from 1 to a few (<10), and thus counting the number of molecules can be equivalent to counting the discrete signals of each nanostructure. The different signal level of the series of nanostructures forms a nanomosaic pattern, which can be detected.

Similarly, the concentration of analyte, if within the second range, as depicted in FIG. 2A(iii), or the third range, can be determined by digital counting of the number of individual nanostructures in the second and/or third series that have bound the analyte relative to either (i) a remaining number of individual nanostructures in the appropriate series that have not bound analyte or (ii) a total number of nanostructures in the corresponding second and/or third series. In other words, the concentration of analyte in a sample across both the first concentration range, the second concentration range, and the optional third (or more) concentration range is determined from a number of individual nanostructures in each of the first series, the second series, and/or the optional third (or more) series that have bound molecules of the analyte.

Alternatively or in addition, the concentration of analyte, if within the second concentration range or the optional third concentration range, can be determined by analog detection of a substantially uniform change in an optically detectable property of the nanostructures in the second region and/or the third region as a function of the concentration of the analyte. For example, the change in the optically detectable property can be a substantially uniform color change created by the second series and/or the optional third series as a function of the concentration of the analyte. In other words, the concentration of analyte in a sample across both the second concentration range and optional third (or more) concentration range(s) is determined by analog detection of a substantially uniform change in an optically detectable property of the nanostructures in each of the second region and/or the third region.

Each individual series (or region) of nanostructures may comprise binding sites for up to 10,000 molecules of the analyte of interest. Each region has a precalibrated continuous signal scale (analog scale) that relates to the number of proteins captured by the region. The analog scale for each region corresponds to a gradual change of physical signal for readout. Different scales may correspond to, for example, different colors from each region under a detector (for example, an optical detector). The region defines a nanomosaic that has a continuum of a property change (for example, color change) as a function of analyte concentration. In the case of optical detection, for example, the different scales may relate to one or more of (i) a light intensity of the region under a microscope which has a continuum of intensity change as a function of concentration or (ii) an electronic measurement, e.g., a current or voltage signal of each region, which has a continuum of current or voltage signal as a function of concentration.

It is contemplated that the nanostructures in a given series can be planar-faced and/or curve-faced nanostructures. The nanostructures can be disposed upon a planar support and/or a flexible substrate, where the nanostructures can be integral with the planar support and/or the flexible substrate. The nanostructures can be fabricated from a semi-conductive material (e.g., silicon) or a metal.

It is contemplated that the sensor may further comprise a fiducial marker, e.g., a fiducial marker that is optically detectable by light field microscopy and/or dark field microscopy. The fiducial marker can be used to calibrate the location of the sensors within the field of detection by the detection system. The sensor may also contain one or more nanostructure fabrication controls that demonstrate, e.g., that the nanostructures fabricated show a change in color as a function of the diameter of the nanostructures.

In another exemplary sensor, as depicted in FIG. 2A(i), the sensor comprises a first region comprising a first series of nanostructures capable of binding the analyte and producing a detectable signal indicative of a concentration of the analyte in the sample within a first concentration range, wherein individual nanostructures of the first series that bind the analyte are optically detected upon binding the analyte, whereupon the concentration of analyte in the sample, if within the first concentration range, is determined from a number of individual nanostructures in the first series that have bound molecules of analyte. The first region of the sensor optionally comprises one or more of: (i) center-to-center spacing of adjacent nanostructures of at least 1 μm; (ii) a minimum cross-sectional dimension or diameter of each nanostructure of at least 10 nm; (iii) a maximum cross-sectional dimension or diameter of each nanostructure of no more than 200 nm; or (iv) a height of each nanostructure in a range of 50 nm to 1000 nm. The sensor optionally further comprises a second region comprising one or more of a (i) a fiducial marker or (ii) a nanostructure fabrication control feature.

In another exemplary sensor, as depicted in FIG. 2A(ii), the sensor comprises a first region comprising a first series of nanostructures capable of binding the analyte and producing a detectable signal indicative of a concentration of the analyte in the sample within a first concentration range, wherein the concentration of analyte in the sample, if within the first concentration range, is determined by analog detection of a substantially uniform change in an optically detectable property of the nanostructures in the first region as a function of the concentration of the analyte. The first region further comprises one or more of: (i) center-to-center spacing of adjacent nanostructures of at least 1 μm; (ii) a minimum cross-sectional dimension or diameter of each nanostructure of at least 100 nm; (iii) a maximum cross-sectional dimension or diameter of each nanostructure of no more than 300 nm; or (iv) a height of each nanostructure in a range of 50 nm to 1000 nm. The sensor optionally further comprises a second region comprising one or more of (i) a fiducial marker or (ii) a nanostructure fabrication control feature.

The sensing region of the disclosed sensors is the physical spot that interacts with biological analytes. In certain embodiments, the sensing region is divided into different parts, with each part targeting a specific concentration range. At very low concentrations, an array of single molecule nanostructures can be used. If analytes are captured by the single molecule sensor, the sensor produces a digital “yes” signal, and thus, the concentration of molecules can be related to the counts of digital sensors. At low-to-medium concentration ranges, a larger nanostructure that has a certain dynamic range to produce an analog signal is used to measure the concentration of analytes. The read-out signal can be resonance spectrum associated with the nanostructure, or scattering intensity, etc. To improve the detection accuracy, an array of these sensors may be used to achieve a statistical average.

As a non-limiting example, the sensing area of a sensor may be divided into multiple regions. By way of example, FIG. 2B is a schematic illustration of a sensor 30 with four sensor regions 32, 34, 36, 38. Each region comprises a series of nanostructures 20. In one embodiment, the series of nanostructures 20d of the ultra-low concentration sensor region 32 define a single molecule sensitivity. As a result, the concentration of analytes correlates with the number of single molecule nanostructures 20d that flip to produce a detectable signal, for example, a “yes” digital signal. The nanostructures 20a of the low, medium and high concentration sensor regions 34, 36, 38 have increasing size and, therefore, lower sensitivities but increasingly larger dynamic ranges. Each of the regions 32, 34 36, 38 are optimized for a specific dynamic range. Together, the results obtained from each region can be aggregated to provide a dynamic range that results from an aggregation of the dynamic ranges achievable by regions 32, 34, 36, 38.

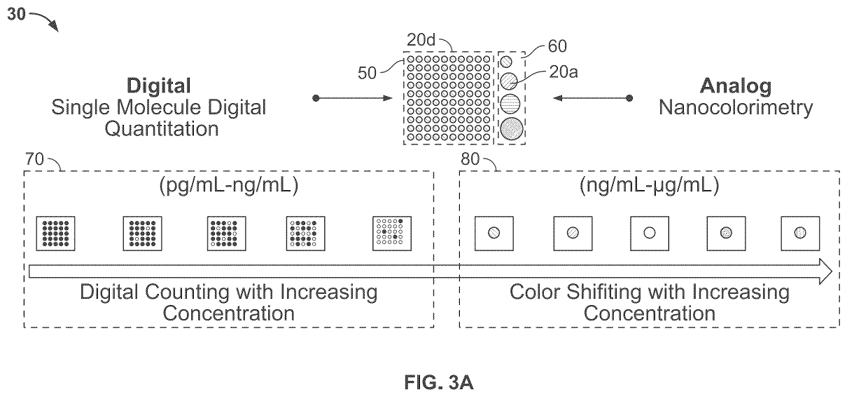

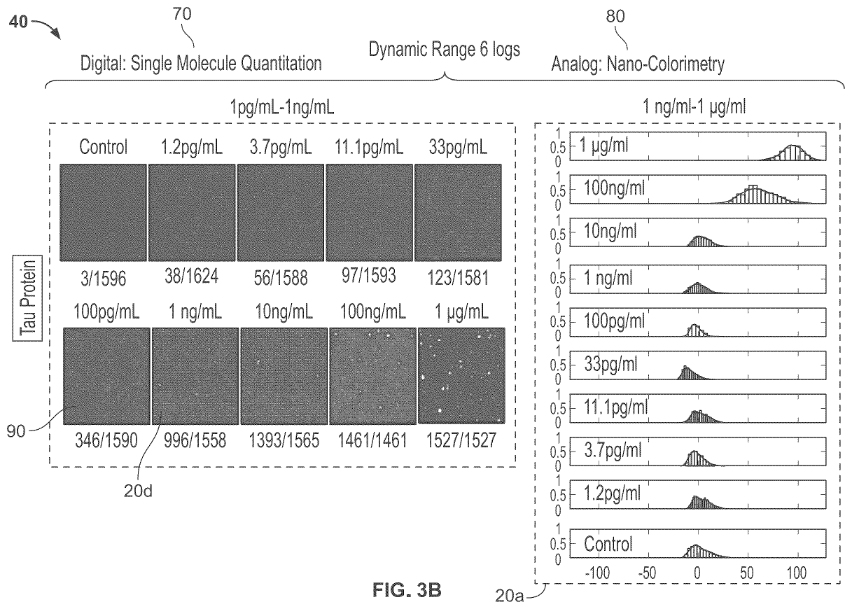

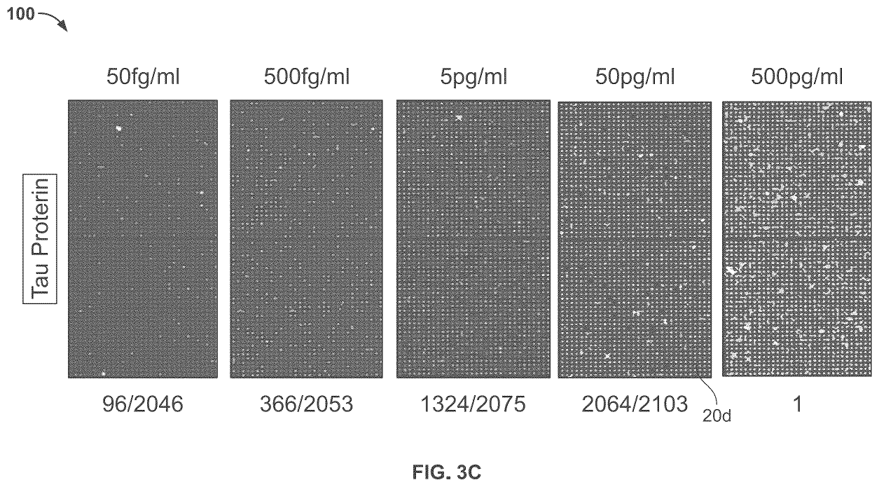

FIG. 3A depicts a schematic representation of an exemplary sensor and the quantification of an analyte of interested achieved using such a sensor. This sensor 30 includes a first region 50 with a series of nanostructures 20d configured for digital quantification and a second region 60 with a series of nanostructures 20a configured for analog quantification where shifts in color indicate different concentrations. In this example, digital quantification 70 is performed for analyte concentrations ranging from pg/mL to ng/mL, and analog quantification 80 is performed for analyte concentration ranging from ng/mL to μg/mL. When concentrations of analyte are in the range of pg/mL to ng/mL, the analyte concentration can be measured based on the number of nanostructures in the series in region 50 that change state (e.g., flip from one state to another). However, as the concentrations of analyte reach the upper limits of the detectable range, the sensor in region 50 becomes saturated and the sensor cannot quantify higher concentrations of analyte. Saturation of the first series may occur when at least 60%, 70%, 80%, 90%, 95%, or greater of the binding sites have bound an analyte. A s a result, this sensor 30 also includes a plurality of series of nanostructures that change their optical properties (for example, detected as a color change) when the concentration of analyte in the sample falls within the range of analyte concentrations that is detectable by a given series of nanostructures. In this embodiment, the series of nanostructures in region 60 are calibrated to change their optical properties (for example, color) in adjacent or overlapping concentration ranges.

In FIG. 3B, sensor 40 includes a series of nanostructures for digital detection/quantification 70 and a series of nanostructures for analog detection/quantification 80. In particular, the series of nanostructures for digital detection 70 comprises nanostructures 20d in the form of an array. As the concentration of analyte (e.g., Tau protein) increases from 1.2 pg/mL to 10 ng/mL, the number of nanostructures that have flipped from one state another increases, as indicated by the ration under each panel 90. At analyte concentrations at or above 10 ng/mL, the series of nanostructures saturates as all or substantially all of the nanostructures (for example, at least 60%, 70%, 80%, 90%, 95% of the binding sites have bound analytes) have flipped from one state to the other. The right-hand side box illustrates the change in optical properties (e.g., colorimetric change) in a series of nanostructures 20a configured for analog detection 80. For example, as the concentration of analyte increases up to 10 ng/mL, the change in optical property (for example, color hue) of the series of nanostructures does not shift. However, as the concentration of analyte is greater than 10 ng/mL, a change in an optical property of the series of nanostructures becomes detectable, for example, as a change in color as a function of analyte concentration. Greater dynamic ranges can be achieved by including in a sensor additional series of nanostructures (for example, digital arrays and/or analog arrays) calibrated to detect and quantify analyte in other concentration ranges.

FIG. 3C illustrates digital quantification performed by a sensor 100 described herein. As illustrated, the sensor is able to detect analyte molecules (molecules of Tau protein) at a concentration 50 fg/mL, with 96 out of 2046 digital nanostructures (20d) being flipped from one optical property to another that is detectable by a detector. In this particular example, the sensor 100 becomes saturated at molecule concentrations at about 50 pg/mL, when all or substantially all of the nanostructures are flipped from one optical state to the other.

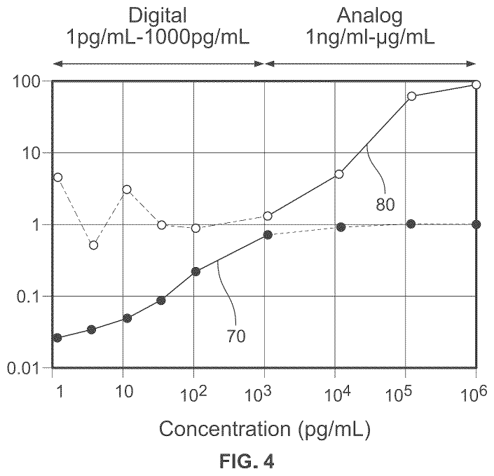

FIG. 4 is a graph depicting data compiled from measurements obtained by the exemplary sensor 40 of FIG. 3B. In the analyte concentration range of 1 pg/mL to 1 ng/mL, the digital quantification mode 70 provides high sensitivity and a dynamic range of 3 logs. In the analyte concentration range of 1 ng/mL to 1 μg/mL, the analog colorimetric measurement 80 extends the detectable concentration range by an additional 3 logs. The transition between the digital quantification measurements and analog quantification measurements to form a continuous curve spanning the entire dynamic range can be automated using an algorithm of the type described herein. In this example, a 6 log dynamic range is achieved using a combination of a series of nanostructures configured for digital quantification with a series of nanostructures configured for analog quantification. It has been discovered that the sensors described herein can achieve large dynamic ranges (for example, 6 logs or more) with high sensitivity (for example, 50 fg/mL) using small volumes of sample (for example, less than 100 μL, 50 μL, 25 μL, 10 μL or 5 μL).

The nanostructure may have any suitable shape and/or size. In some cases, for example, the nanostructure may be a nanoneedle, a nanowire, a nanorod, a nanocone, or the like. Other shapes are also possible, e.g., nanoribbons, nanofilaments, nanotubes, or the like. In certain embodiments, the nanostructures are vertically aligned, although other angles or alignments are also possible. Nanostructures such as nanoneedles, nanodots, nanodisks, nanopillars, etc. have single molecule level sensitivity due to their ability to confine electromagnetic energy through coupling to surface polaritons.

The physical form of a sensor may be an array or matrix of nanostructures, for example, nanoneedles, nanowires, nanopillars, nanodots, etc., fabricated on a surface by bottom-up and/or top-down methods. The surface can be a flat surface, such as a top surface of a wafer. Alternatively, the surface may also be curved or flexible, or part of a three dimensional structure such as a fiber or a wire or the like.

The functional form of the sensor can comprise nano-optical structures, nanomechanical structures or nano-electrical structures. Accordingly, the read-out signal includes but is not limited to optical signals, electrical signals and mechanical signals. Accordingly, the concentration of the analytes may be determined by changes in optical, electrical or nanomechanical properties of the nanostructures. The optical features include, for example, surface plasmon resonance, nanophotonic resonance, electric resonance, magnetic resonance, scattering, absorption, fluorescence, color changes, or the like. The electrical features include, e.g., resistance, capacitance, current, voltage, or the like. The nanomechanical features include, for example, vibrational resonance, vibration magnitude, mechanical mass, or the like.

The foregoing structures may also be used to detect high concentration of analytes by observing changes in their optical properties, for example, surface plasmon resonances, scattering intensities, or absorptions. Sensitivity and detection ranges of these structures are closely related to the sizes of the structures. Planar fabrication technology enables scalable and flexible integration of differently sized and shaped nanostructures in one device. Different nanostructures may be used to achieve high sensitivity and a high dynamic range for the determination of molecules and analytes in a biological sample.

In certain embodiments, the surface properties of different structures can be designed such that the nanostructures in a first series of nanostructures may have higher binding affinities for binding the analyte than that of the second and/or third series of nanostructures. This can be achieved using binding agents having different binding affinities to a given analyte. As a result, at low concentrations, analytes are preferentially captured and detected by the single molecule nanostructures. As the concentration increases, the nanostructures of the first series saturate and signals from other series of nanostructures can be used to extend the dynamic range.

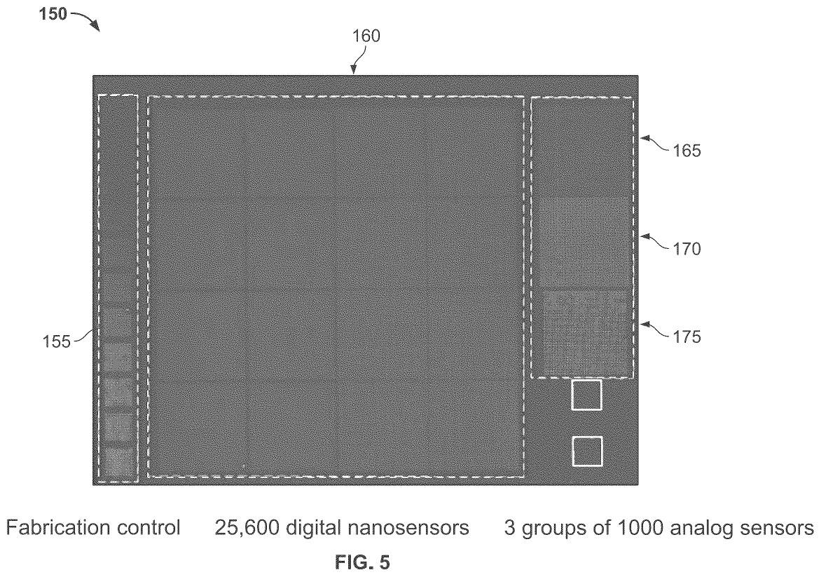

FIG. 5 is a pictorial representation of an exemplary sensor (for example, a nanomosaic chip) 150 which includes multiple series of nanostructures. In the column on the left hand side of sensor 150, the separate regions represent fabrication control structures 155 which demonstrate that the nanostructures change color as the diameter of the nanostructures is increased. The middle region 160 represents multiple separate arrays (i.e., 16 arrays) each defining a corresponding series of nanostructures (collectively comprising 25,600 nanostructures that each define single molecule nanostructures) configured for digital quantification for measuring ultra-low concentration levels of analytes. The region on the right hand side comprises three series of nanostructures (e.g., a second, third, and fourth series of nanostructures) depicted as regions 165, 170, 175, for analog quantification. Each of the regions 165, 170, 175 are calibrated to measure analyte concentrations within three separate adjacent or overlapping concentration ranges. In certain embodiments, the three regions may each comprise 1,000 nanostructures.

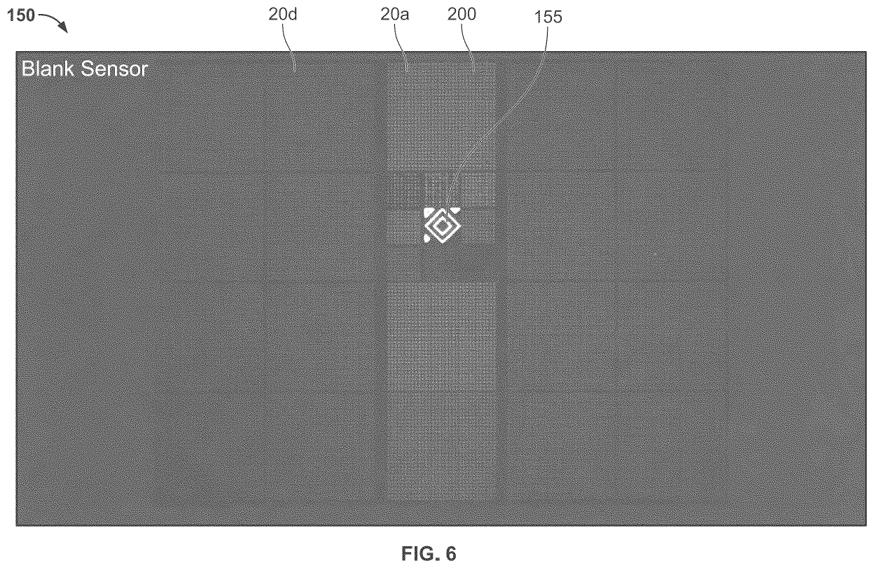

In an alternative embodiment, as shown pictorially in FIG. 6, another exemplary sensor (e.g., a nanomosaic chip) 150 comprises numerous series (regions) of nanostructures. In the center, a fiducial marker 200 is located to assist in aligning the sensor with an optical detection system. The fiducial marker can be any desired design. For example, as shown in FIG. 6, the fiducial marker 200 comprises a diamond pattern and three triangular patterns arranged in a way that does not have rotational symmetry to provide location and rotational orientation information. As a result, the fiducial marker can be used to (i) locate the sensor position, and (ii) align the horizontal and vertical planes of the nanostructures. Fabrication control structures 155 are disposed around the fiducial. Arrays of digital single molecule nanostructures 20d are disposed on the left and the right regions of the sensor, and arrays of analog molecule nanostructures 20a are disposed in the center row surrounding the fiducial and fabrication control structures. The fabrication control shown in FIG. 6 comprises 8 blocks of nanostructures (e.g., nanoneedles) whose diameters range from 80 nm to 150 nm. The color of the nanostructures (nanoneedles) under dark field imaging changes as the diameter increases.

In certain embodiments, the nanostructure has a length, determined from an end or a point of attachment with a substrate, of less than about 500 nm, 450 nm, 350 nm, 300 nm, 250 nm, 200 nm, 150 nm, 100 nm, 50 nm, 30 nm, 20 nm, 10 nm, 5 nm, 3 nm, or 2 nm. In certain embodiments, the length of the nanostructure may be at least about 2 nm, 3 nm, 4 nm, 5 nm, 6 nm, 6 nm, 7 nm, 8 nm, 9 nm, 10 nm, 20 nm, 30 nm, 40 nm, 50 nm, 60 nm, 70 nm, 80 nm, 90 nm, 100 nm, 150 nm, 200 nm, 250 nm, 300 nm, 350 nm, 400 nm, 450 nm, or 500 nm.

The nanostructure may have any suitable cross-sectional shape, for example, square, circular, triangular, ellipsoidal, polygonal, star, irregular shape, etc. The nanostructure may maintain the same cross-sectional shape throughout its length, or may have different cross-sectional shapes in different portions of the nanostructure. In addition, the nanostructures may have any suitable cross-sectional diameter. The cross-sectional diameter may be constant (e.g., as in a nanoneedle or a nanorod), or varying (e.g., as in a nanocone). The average cross-sectional diameter may be, for example, less than about 1,000 nm, 750 nm, 500 nm, 400 nm, 300 nm, 200 nm, 175 nm, 150 nm, 125 nm, 100 nm, 75 nm, 50 nm, 40 nm, 30 nm, 20 nm, or 10 nm. In certain embodiments, the cross-sectional diameter may be at least about 10 nm, 20 nm, 30 nm, 40 nm, 50 nm, 75 nm, 100 nm, 125 nm 150 nm, 175 nm, 200 nm, 300 nm, 400 nm, 500 nm, 750 nm, or 1,000 nm. Combinations are also possible in various embodiments. For example, the average diameter of the nanostructures may be between 50 nm and 300 nm, 75 nm and 250 nm, or 100 nm to 200 nm.

(B) Fabrication Considerations

The nanostructure may be formed out of any suitable material, and may be the same or different from a substrate upon which it is disposed. In certain embodiments, the nanostructures (e.g., nanoneedles) can be formed from silicon and/or other suitable semi-conductive materials (e.g., germanium). Additional, non-limiting examples of materials include metals (e.g., nickel or copper), silica, glass, or the like.

In certain embodiments, the nanostructure (e.g., nanoneedle) may be disposed on a substrate can be formed from a unitary material. In other words, the nanostructure (e.g., nanoneedle) and the underlying substrate (e.g., planar substrate) maybe unitary and may be formed from the same material. In other approaches, the nanostructure (e.g., nanoneedle) maybe bonded or adhered to an underlying substrate (e.g., planar substrate), which may be formed from the same material or from different materials.

It is contemplated that the sensors described herein can be fabricated by a number of different approaches, for example, using semiconductor manufacturing approaches. A s discussed above and in more detail below, any suitable method can be used to form the series of nanostructures useful in creating the sensors described herein. Examples include, but are not limited to, lithographic techniques such as e-beam lithography, photolithography, X-ray lithography, extreme ultraviolet lithography, ion projection lithography, etc. Alternatively or in addition, the nanostructure may be formed from one or more materials that are susceptible to etching with a suitable etchant.

For example, in certain embodiments, the nanostructures may be formed from one or more materials that are susceptible to etching with a suitable etchant. For instance, the nanostructures may comprise materials such as silica or glass, which can be etched using HF (hydrofluoric acid) or BOE (buffered oxide etch). As another example, the nanostructures may comprise a metal such as copper, iron, nickel, and/or steel, which can be etched using acids such as HCl (hydrochloric acid), HNO₃ (nitric acid), sulfuric acid (H₂SO₄), and/or other etching compounds such as such as ferric chloride (FeCl₃) or copper sulfate (CuSO₄). As yet another example, the nanostructures may comprise silicon or other semiconductor materials, which can be etched using etchants such as EDP (a solution of ethylene diamine and pyrocatechol), KOH (potassium hydroxide), and/or TMAH (tetramethylammonium hydroxide). The nanostructures may also comprise, in some cases, a plastic or a polymer, e.g., polymethylmethacrylate, polystyrene, polyperfluorobutenylvinylether, etc., which can be etched using KOH (potassium hydroxide), and/or other acids such as those described herein.

(i) Nanostructure Fabrication

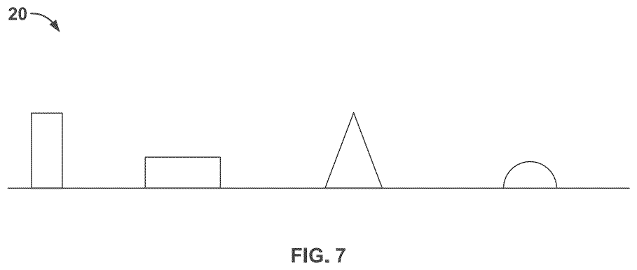

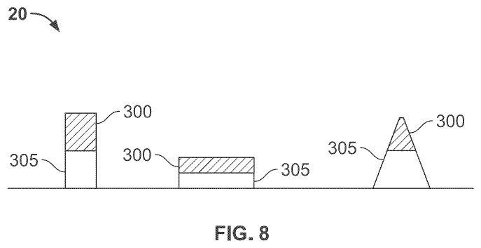

It is contemplated that the sensors described herein can be fabricated by conventional semiconductor manufacturing technologies, for example, CMOS technologies, that have led to high manufacturing capacity, at high throughputs and yields in a cost-effective manner. Using such approaches it is possible to make sensors containing one of more series of nanostructures, e.g., nanoneedles, nanodots, nanodisks, nanowires, and nanopillars disposed upon or integral with a substrate. Exemplary nanostructures are depicted schematically in FIGS. 7 and 8. As non-limiting examples, FIG. 7 illustrates several nanostructures 20 that can be directly formed on a substrate with current nanofabrication technologies, including electron beam lithography, photolithography, nanoimprinting, etc. For example, the nanostructure 20 can be a nanopillar (a uniform nanoneedle), a nanodisk, a cone-shaped nanoneedle, or a nanodot. In addition, FIG. 8 depicts nanostructures 20 (e.g., nanoneedles) fabricated from two or more materials, e.g., first and second materials 300 and 305, respectively. The compositions of each material can be used to control the binding capacity of the nanostructures for binding analyte or to achieve specific optical, electrical, or magnetic properties, as discussed below.

The fabrication of nanostructures may be performed either at wafer scale or at chip scale with equivalent scaling capability. In this type of approach, a mask is first made for the designed nanostructure. In certain embodiments, an inverse to the design structure is used as the pattern on the mask. For example, a photoresist is coated onto the wafer or on the chip, for example, using a spin-coating or dip-coating process. The photoresist may then be exposed to electromagnetic radiation through the mask to the photoresist. Thereafter, the exposed photoresist is developed. In certain embodiments, the pattern on the photoresist can also be directly written by means of a laser beam or an electron beam. The pattern on the photoresist can then be transferred to the substrate by physical vapor deposition, including thermal evaporation, electron beam evaporation, sputter or chemical deposition, or atomic layer deposition of a desired material.

In certain embodiments, the pattern on the photoresist can be transferred to the substrate using top down etching process, including wet etching, dry etching such as reactive ion etching, sputter etching, and/or vapor phase etching. The patterning, deposition, etching, and functionalization processes can be repeated for multiple cycles. In certain embodiments, arrays of nanoneedles, nanopillars, nanodots and/or nanowires can be fabricated using semiconductor manufacturing processes. In other embodiments, arrays of nanoneedles, nanopillars, nanodots and/or nanowires can be fabricated using mold-stamping process.

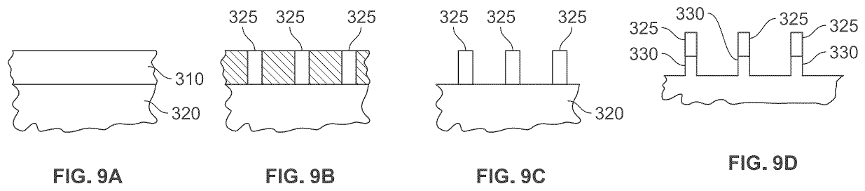

An exemplary fabrication approach is depicted in the cross-sectional views shown in FIGS. 9A-9D. Referring to FIG. 9A, more specifically, a layer of ebeam resist or photoresist 310 is coated onto a semiconductor substrate 320, such as a silicon substrate. Referring to FIG. 9B, the resist layer is then patterned by electron beam exposure or electromagnetic radiation exposure to form resist layer features 325, for example, by using an Elionix or Raith electron beam lithography system. Referring to FIG. 9C, the resist is developed in resist developer, to remove portions thereof and leaving only the resist features 325. Referring to FIG. 9D, an etching process is then performed with the patterned resist serving as a mask. The etching process may be, e.g., a wet or a dry etch. A suitable wet etch can be, for example, a solution of ethylenediamine pyrocatechol (EDP), potassium hydroxide (KOH), or tetramethylammonium hydroxide (TMAH). As a result, silicon nanoneedles 330 are created with resist 325 disposed upon the top surface of the nanoneedles. The height of the nanoneedles can range from 2 nm to 1000 nm. The diameter of the nanoneedles can range from 10 nm to 1000 nm. Resist features 325 may be removed using a conventional wet etching buffer (not shown).

The surface of the etched structure can be chemically activated using chemical vapor deposition or atomic layer deposition or a hybrid of both. This activation process can also be performed in a wet solution. The chemically activated structure is then ready to bind a biological material, a binding agent described herein via, for example, chemisorption (e.g., covalent binding) or physisorption.

A suitable silicon substrate can be, for example, a round 12″ silicon wafer. In order to comply with Society of Biomolecular Screening (SBS) recommended microplate specifications, the round wafer is diced into a rectangular shape. The dicing step can be performed at the end of the fabrication process as described above. Alternatively, dicing into half of the depth of the wafer can be performed in the beginning of the fabrication process; then, after completion of all fabrication steps (including spin coating, patterning, deposition and etching), the wafers can be easily cleaved into the SBS format.

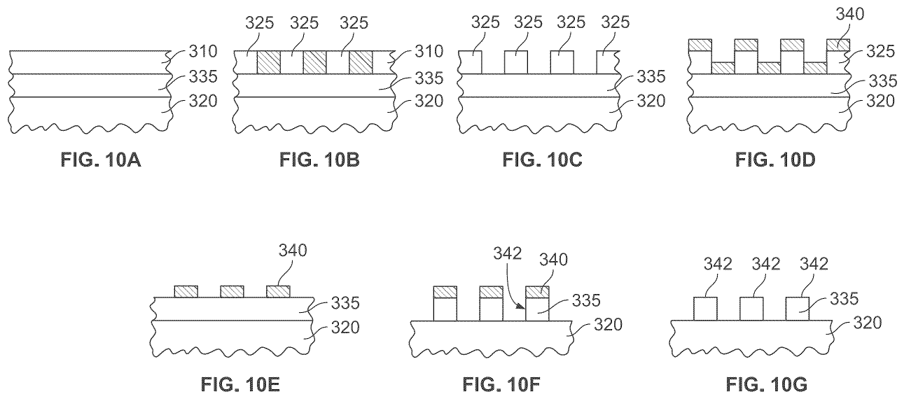

Another fabrication approach is depicted in the cross-sectional views shown in FIGS. 10A-10G. Referring to FIG. 10A, a silicon dioxide layer 335 is formed on a top surface of a silicon substrate 320 using chemical vapor deposition, atomic layer deposition or a combination of both. The thickness of the layer can range from 2 nm to 100 nm. A resist layer 310 comprising, e.g., polymethyl methacrylate, is spun coated onto the silicon dioxide layer 335. Referring to FIGS. 10B and 10C, the resist layer 310 is patterned by an electron beam or electromagnetic radiation, and then developed in resist developer to form resist features 325. Referring to FIG. 10D, an aluminum layer 340 is deposited over the patterned resist layer features 325 by, e.g., thermal evaporation (or electron evaporation) with, e.g., a Sharon thermal evaporator or Denton e-beam evaporator. The aluminum layer 340 is preferably 20 nm to 100 nm thick. Referring to FIG. 10E, a lift-off process is performed to remove the resist layer features 325, leaving behind an aluminum mask over the silicon dioxide layer 335. Referring to FIG. 10F, an etching process, such as a reactive ion etch with an STS ICP RIE system or an Oxford plasma RIE system is performed to etch silicon oxide nanoneedles 335. The RIE etching can further proceed into the silicon layer 320, resulting in a two layer SiO2-Si nanostructures. Referring to FIG. 10G, the aluminum mask 340 may be etched off the tops of silicon nanoneedles 342 in an aluminum etchant buffer, e.g., a mixtures of 1-5% HNO₃, H₃PO₄ and CH₃COOH.

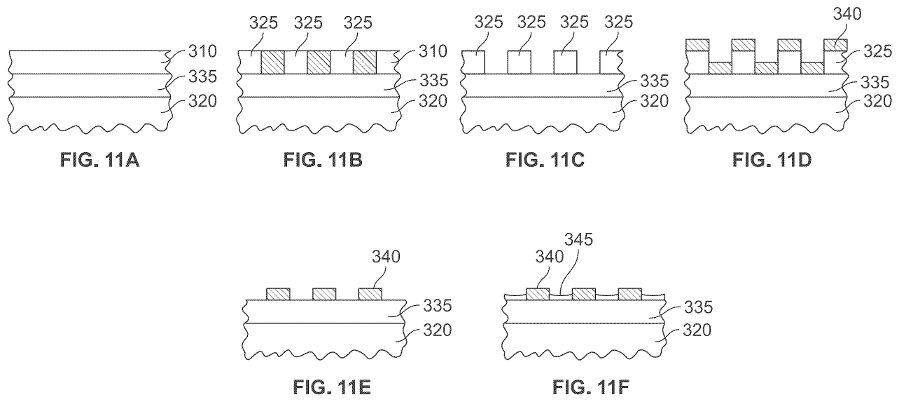

Yet another fabrication approach is depicted in the cross-sectional views shown in FIGS. 11A-11F. Referring to FIG. 11A, a silicon dioxide layer 335 is grown on a top surface of a silicon substrate 320. A resist layer 310 is spun coated onto the silicon dioxide layer 335. Referring to FIGS. 11B and 11C, the resist layer 310 is patterned by electron beam or electromagnetic radiation, and then developed in resist developer to form resist features 325. Referring to FIG. 11D, a metal layer, such as an aluminum layer 340, is deposited over the patterned resist layer 310 by, for example, a thermal evaporation (or electron evaporation) process. Referring to FIG. 11E, a lift-off process is then performed to remove the resist layer 310, leaving behind aluminum nanoneedles disposed upon the oxide layer on the substrate. Referring to FIG. 11F, a coating layer 345 can be spun coated to modify the surface properties of the substrate. The coating layer can be a hydrophobic material, such as TEFLON, or a layer of polyethylene glycol molecules. The thickness of the coating layer is smaller than the height of the aluminum nanoneedles.

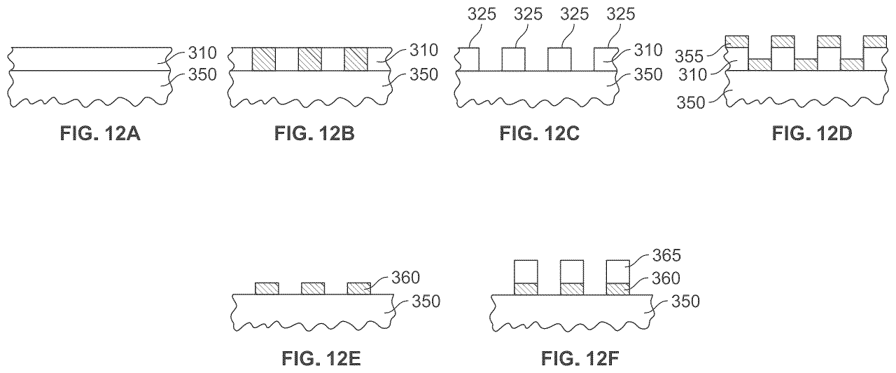

Another fabrication approach is depicted in the cross-sectional views shown in FIGS. 12A-12F. Referring to FIG. 12A, a resist layer 310 is spun coated on an oxide substrate 350. The oxide layer can be a thermally grown silicon oxide, or formed by chemical vapor deposition. In some embodiments, the substrate 350 may be a glass slide. Referring to FIGS. 12B and 12C, electromagnetic radiation can be used to pattern features in the resist layer 310, which is then developed in resist developer to form resist features 325. Referring to FIG. 12D, a silicon layer 355 is deposited over the patterned resist layer 310 by, for example, using chemical vapor deposition. Referring to FIG. 12E, a lift-off process is performed to remove the patterned resist layer 310, which results in a silicon nanodot 360 structure on the oxide substrate. Referring to FIG. 12F, silicon nanoneedle structures 365 may be epitaxially grown using the silicon nanodots 360 as seeds, by, e.g., VLS (vapor-liquid-solid) method.

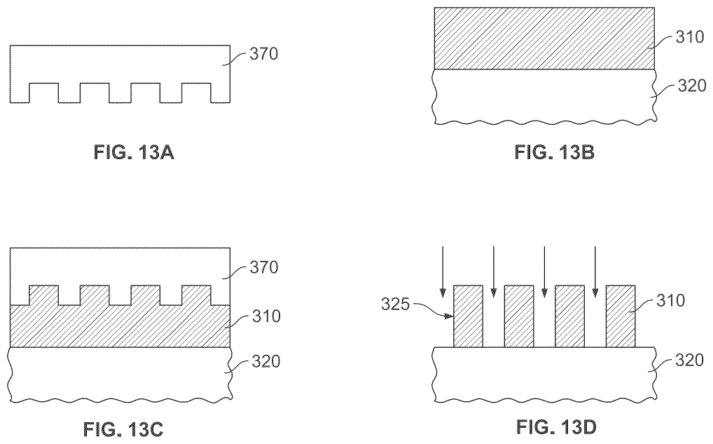

Another fabrication approach is depicted in the cross-sectional views shown in FIGS. 13A-13D, in which a photoresist layer may be patterned by using a mold. Referring to FIG. 13A, a mold 370 is made from e.g., Si or quartz. The mold can be made by high resolution patterning technology, such as ebeam lithography. The mold has feature sizes similar to that of the target nanostructures to be replicated. Referring to FIG. 13B, a resist layer 310 is spun coated on silicon substrate 320. Referring to FIG. 13C, the features in mold 370 are then stamped into the resist by nanoimprinting or nanostamping, and then crosslinked by e.g., UV or heat. Referring to FIG. 13D, the imprinted photoresist can be used as the mask for the subsequent etching process to obtain the silicon nanostructures.

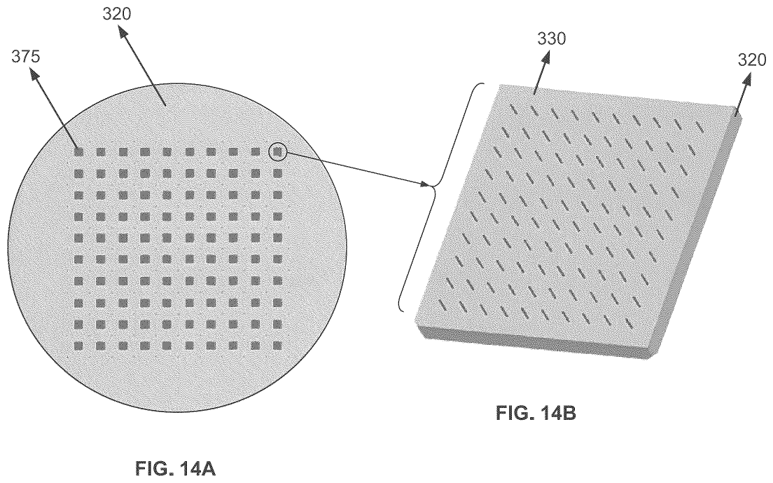

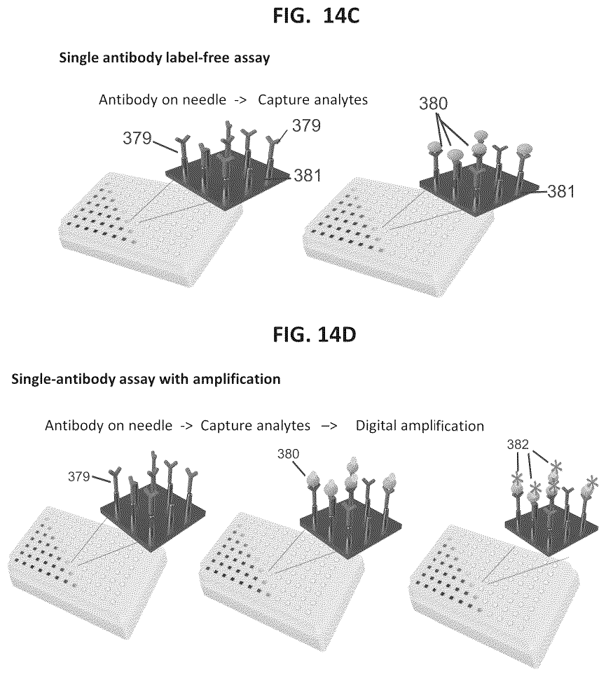

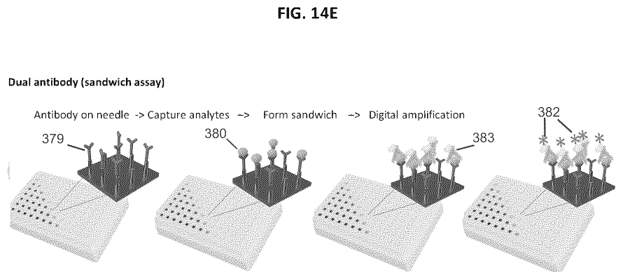

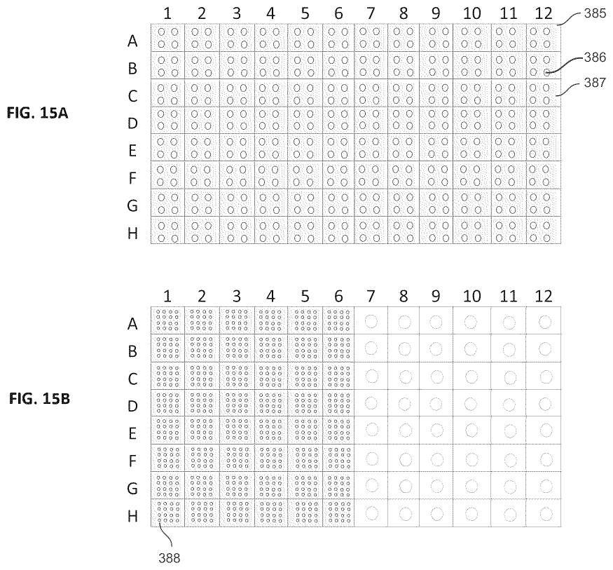

With reference to FIGS. 14A and 14B, by replicating the fabrication steps described hereinabove it is possible to produce a plurality of sensors 375 fabricated on a wafer 320, to create, for example, a 10×10 array of sensors disposed on each wafer 320. As shown in FIG. 14B, each sensor comprises an array of nanostructures, e.g., nanoneedles 330 disposed upon a silicon substrate.

It should be noted that the nanostructures depicted in FIGS. 10-14 have at least one dimension in the range of 1-999 nm, 1-750 nm, 1-500 nm, 1-400 nm, 1-300 nm, 1-200 nm, 1-100 nm, 10-999 nm, 10-750 nm, 10-500 nm, 10-400 nm, 10-300 nm, 10-200 nm, 10-100 nm, 20-999 nm, 20-750 nm, 20-500 nm, 20-400 nm, 20-300 nm, 20-200 nm, 20-100 nm, 30-999 nm, 30-750 nm, 30-500 nm, 30-400 nm, 30-300 nm, 30-200 nm, 30-100 nm, 40-999 nm, 40-750 nm, 40-500 nm, 40-400 nm, 40-300 nm, 40-200 nm, 40-100 nm, 50-999 nm, 50-750 nm, 50-500 nm, 50-400 nm, 50-300 nm, 50-200 nm, or 50-100 nm. The pitch, i.e., center-to-center distance, between nanostructures, for example in FIG. 14B, is typically 1-100 μm, for example, at least 1.5 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, or 90 μm. Other dimensions may be used for the pitches of the structures. The array of nanostructures in FIG. 14B, in its entirety, can also be arranged in an array format, as shown in FIG. 14A. For example, the pitch in between two arrays of nanostructures, shown in FIG. 14A may range from less than 100 μm to larger than a few centimeters. Furthermore, it is contemplated that the pitch and size of the nanostructures may be different in different parts of the chip, or within each series of nanostructures. Combinations of any of these are also possible in various embodiments.

Furthermore, the distance or pitch between nanostructures in a periodic structure may be controlled, for example, such that the nanostructures form a meta-surface. For example, the pitch may be set to be less than the wavelength of the incident light. For instance, the pitch may be less than 700 nm, 600 nm, 500 nm, 400 nm, 300 nm, 200 nm, 100 nm, 50 nm, 25 nm, 10 nm, 9 nm, 8 nm, 7 nm, 6 nm, 5 nm, 4 nm 3 nm or 2 nm, and/or greater than 1 nm, 2 nm, 3 nm, 4 nm, 5 nm, 6 nm, 7 nm, 8 nm, 9 nm 10 nm, 25 nm, 50 nm, 100 nm 200 nm, 300 nm, 400 nm, 500 nm, 600 nm or 700 nm. For example, under certain circumstances, the pitch may be between 400 nm and 500 nm. The nanostructures may have any of the dimensions provided herein. Under certain circumstances, the average cross-sectional diameter or minimum or maximum cross-sectional dimension of the nanostructure is less than the wavelength of the incident light. Under certain circumstances, the individual nanostructures are configured to be optically resolvable, where, for example, the pitch may be less than 100 μm, less than 10 μm, less than 5 μm, and/or greater than 1 μm, or greater than 5 μm.

Table 1 describes exemplary parameters of the nanostructures described herein for optical read-outs.

TABLE 1

| | Minimum | Typical Value or | Maximum | |
|---|---|---|---|---|
| Parameter | Value | Range | Value | Units |
| Digital nanostructure cross-sectional | 10 | 60-95 | 150 | nm |
| dimension or diameter | | | | |
| Analog nanostructure cross-sectional | 100 | Depends on analyte | 1,000 | nm |
| dimension or diameter | | concentration (e.g., | | |
| | | can be 110-130) | | |
| Center-center spacing of adjacent | 1 | 1.5-3 | Depends on | μm |
| nanostructures | | | substrate | |
| | | | size | |
| Height of the nanostructure | 50 | 100-250 | 1,000 | nm |

Table 2 describes exemplary parameters of the nanostructures described herein for a mechanical read-out.

TABLE 2

| | Minimum | Typical Value or | Maximum | |
|---|---|---|---|---|
| Parameter | Value | Range | Value | Units |
| Digital nanostructure cross-sectional | 0.1 | 60-95 | 100 | nm |
| dimension or diameter | | | | |
| Analog nanostructure cross-sectional | 100 | Depends on analyte | 100,000 | nm |
| dimension or diameter | | concentration | | |
| Center-center spacing of adjacent | 10 | 10-100 | Depends on | μm |
| nanostructures | | | substrate | |
| | | | size | |
| Height of the nanostructure | 50 | 100-1,000 | 10,000 | nm |

Table 3 describes exemplary parameters of the nanostructures described herein for an electrical read-out.

TABLE 3

| | Minimum | Typical Value or | Maximum | |
|---|---|---|---|---|
| Parameter | Value | Range | Value | Units |
| Digital nanostructure cross-sectional | 5 | 10-100 | 500 | nm |
| dimension or diameter | | | | |
| Analog nanostructure cross-sectional | 100 | Depends on analyte | 1000 | nm |
| dimension or diameter | | concentration | | |
| Center-center spacing of adjacent | 10 | 100-1,000 | Depends on | μm |
| nanostructures | | | substrate | |
| | | | size | |
| Height of the nanostructure | 10 | 100-500 | 10,000 | nm |

(ii) Nanostructure Functionalization

The nanostructures in the first series and, where applicable, the second and third series, are functionalized with a binding agent that binds the analyte, for example, binding agent, for example, a biological binding agent, that binds the analyte. The biological binding agent can be, for example, an antibody, an aptamer, a member of a ligand-receptor pair, an enzyme, or a nucleic acid. Under certain circumstances, for example, when the first series is used to measure very low concentrations of analyte, it may be advantageous to use a binding agent in the first series that has a higher binding affinity for the analyte than the binding agent in a second, third or subsequent series.

The number of binding agents applied to a given nanostructure may vary depending upon the desired assay, for example, the required dynamic range, number of analytes to be detected, etc. For example, under certain circumstances, a nanostructure may be functionalized with 1, 5, 10, 20, 25, 50, 75, 100 or more binding agents. These values may range from 1-1,000, 1-500, 1-250, 1-100, 1-50, 1-25, 1-10 or 1-5 binding agents per nanostructure.

The sensor may be designed to detect and/or quantify any analyte of interest in a sample. Furthermore, a nanostructure or series of nanostructures in a given sensor may be configured to bind, detect and/or quantify plurality of different analytes simultaneously or sequentially. For example, the sensor can comprise a plurality of different binding agents for detecting a corresponding plurality of different analytes in the test sample.

Analytes may be detected and/or quantified in a variety of samples. The sample can be in any form that allows for measurement of the analyte. In other words, the sample must permit analyte extraction or processing to permit detection of the analyte, such as preparation of thin sections. Accordingly, the sample can be fresh, preserved through suitable cryogenic techniques, or preserved through non-cryogenic techniques. In certain embodiments, the sample is a body fluid sample, such as a blood, serum, plasma, urine, cerebrospinal fluid, or interstitial fluid sample. In certain embodiments, the sample is a tissue extract obtained, for example, from a biopsy sample obtained by using conventional biopsy instruments and procedures. Endoscopic biopsy, excisional biopsy, incisional biopsy, fine needle biopsy, punch biopsy, shave biopsy and skin biopsy are examples of recognized medical procedures that can be used by one of skill in the art to obtain tissue samples. Suitable techniques for tissue preparation for subsequent analysis are well-known to those of skill in the art. In certain embodiments, the sample is a cell sample or a cell supernatant sample.

Analytes include biological molecules, for example, a protein which includes a protein, glycoprotein, lipoprotein, nucleoproteins, and a peptide, including a peptide of any one of the foregoing proteins. Exemplary protein-based analytes include, for example and without limitation, cytokines, antibodies, enzymes, growth factors, hormones, structural proteins, transport proteins, receptors, DNA-binding proteins, RNA-binding proteins, immune system proteins, chaperone proteins, etc.

In certain embodiments, the analyte is a cytokine, e.g., an interferon (e.g., IFNα, IFNβ, and IFNγ), interleukin (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In certain embodiments, the analyte is an antibody. Examples of antibodies include, but are not limited to, anti-EGFR, anti-HER2, anti-PD1, anti-PIK3CA, anti-anti-Tau, anti-RhoA, anti-β-actin, anti-α-tubulin, anti-β-tubulin, anti-YAP, anti-TAZ, anti-NRF2, anti-SIRT1, anti-SIRT2, anti-GIRK2, anti-IL-6, anti-IL-9, anti-FLT3, anti-BCMA, anti-ghrelin, anti-oxytocin, anti-prolactin, and anti-relaxin.

In certain embodiments, the analyte is an enzyme. Examples of enzymes include, but are not limited to, nitrite reductase, nitrate reductase, glutathione reductase, thioredoxin reductase, sulfite oxidase, cytochrome p450 oxidase, nitric oxide dioxygenase, thiaminase, alanine transaminase, aspartate transaminase, cysteine desulfurase, lipoyl synthase, phospholipase A, acetylcholinesterase, cholinesterase, phospholipase C, fructose bisphosphatase, phospholipase D, amylase, sucrase, chinitase, lysozyme, maltase, lactase, beta-galactosidase, hyaluronidase, helicase, ATPase, and DNA Polymerase.

In certain embodiments, the analyte is a growth factor. Examples of growth factors include, but are not limited to, Colony-stimulating factors (CSFs), Epidermal growth factor (EGF), Fibroblast growth factor (FGF), Platelet-derived growth factor (PDGF), Transforming growth factors (TGFs), and Vascular endothelial growth factor (VEGF).

In certain embodiments, the analyte is a hormone. Examples of hormones include, but are not limited to, epinephrine, melatonin, norepinephrine, triiodothyronine, thyroxine, dopamine, prostaglandins, leukotrienes, prostacyclin, thromboxane, amylin (or islet amyloid polypeptide), anti-mullerian hormone (or mullerian inhibiting factor or hormone), adiponectin, adrenocorticotropic hormone (or corticotropin), angiotensinogen and angiotensin, antidiuretic hormone (or vasopressin, arginine vasopressin), atrial-natriuretic peptide (or atriopeptin), brain natriuretic peptide, calcitonin, cholecystokinin, corticotropin-releasing hormone, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor (or somatomedin), leptin, lipotropin, luteinizing hormone, melanocyte stimulating hormone, motilin, orexin, osteocalcin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone (or thyrotropin), thyrotropin-releasing hormone, vasoactive intestinal peptide, guanylin, uroguanylin, testosterone, dehydroepiandrosterone, androstenedione, dihydrotestosterone, aldosterone, estradiol, estrone, estriol, cortisol, progesterone, calcitriol (1,25-dihydroxyvitamin D3), and calcidiol (25-hydroxyvitamin D3).

In certain embodiments, the analyte is a structural protein. Examples of structural proteins include, but are not limited to, actin, myosin, catenin, keratin, plakin, collagen, fibrillin, filaggrin, gelatin, claudin, laminin, elastin, titin, and sclerotin.

In certain embodiments, the analyte is a transport protein. Examples of transport proteins include, but are not limited to, EAAT1, EAAT2, EAAT3, EAAT4, EAAT5, glucose transporter, dopamine transporter, norepinephrine transporter, serotonin transporter, vesicular monoamine transporter, ATP-binding cassette transporter, V-type ATPases, P-type ATPases, F-Type ATPases, and rhodopsin.

In certain embodiments, the analyte is a receptor. Examples of receptors include, but are not limited to, G protein coupled receptors, adrenergic receptors, olfactory receptors, receptor tyrosine kinases, Epidermal growth factor receptor (EGFR), Insulin Receptor, Fibroblast growth factor receptors, high affinity neurotrophin receptors, Ephrin receptors, Integrins, low affinity Nerve Growth Factor Receptor, and NMDA receptor.

In certain embodiments, the analyte is a DNA-binding protein. Examples of DNA-binding proteins include, but are not limited to, H1/H5, H2, H3, H4, protamines, and transcription factors (e.g., c-myc, FOXP2, FOXP3, MyoD, p53, etc.).

In certain embodiments, the analyte is an RNA-binding protein. Examples of RNA-binding proteins include, but are not limited to, Serrate RNA effector molecule homolog (SRRT), TAP/NXF1, ZBP1, GLD-1, GLD-3, DAZ-1, PGL-1, OMA-1, OMA-2, Pumilio, Nanos, FMRP, CPEB and Staufen.

In certain embodiments, the analyte is an immune system protein. Examples of immune-system proteins include, but are not limited to, CD34, CD31, CD117, CD45, CD11B, CD15, CD24, CD44, CD114, CD182, CD4, CD8, CD3, CD16, CD91, CD25, CD56, CD30, CD31, CD38, CD47, CD135, and FOXP3.

In certain embodiments, the analyte is a chaperone protein. Examples of chaperone proteins include, but are not limited to, GRP78/BIP, GRP94, GRP170, calnexin, calreticulin, HSP47, ERp29, protein disulfide isomerase (PDI), prolyl isomerase, ERp57, HSP70, HSP90, and HSP100.

The nanostructures can be functionalized using standard chemistries known in the art. As an initial matter, the surfaces of the nanostructures may be activated for binding a binding agent using standard chemistries, including standard linker chemistries.

The binding agent may contain or be engineered to contain a functional group capable of reacting with the surface of the nanostructure (e.g., via silanol groups present on or at the surface of the nanostructure), either directly or via a chemical linker.

In one approach, the surface silanol groups of the nanostructure may be activated with one or more activating agents, such as an alkoxy silane, a chlorosilane, or an alternative silane modality, having a reactive group (e.g., a primary amine). Exemplary alkoxy silanes having a reactive group may include, for example, an aminosilane (e.g., (3-aminopropyl)-trimethoxysilane (APTMS), (3-aminopropyl)-triethoxysilane (APTES), (3-aminopropyl)-diethoxy-methylsilane (APDEMS), 3-(2-aminoethyaminopropyl)trimethoxysilane (AEAPTM)), a glycidoxysilane (e.g., (3-glycidoxypropyl)-dimethyl-ethoxysilane (GPMES)), or a mercaptosilane (e.g., (3-mercaptopropyl)-trimethoxysilane (MPTMS) or (3-mercaptopropyl)-methyl-dimethoxysilane (MPDMS). Exemplary chlorosilanes having a reactive group include 3-(trichlorosilyl)propyl methacrylate (TPM) and 10-isocyanatodecyltrichlorosilane.

Thereafter, a functional group on the binding agent, for example, a primary amine on the side chain on a lysine residue can be attached to the reactive group added to the surface of the nanostructure using a variety of cross-linking agents. Exemplary cross-linking agents can include, e.g., homobifunctional cross-linking agents (e.g., glutaraldehyde, bismaleimidohexane, bis(2-[Succinimidooxycarbonyloxy]ethyl) sulfone (BSOCOES), [bis(sulfosuccinimidyl)suberate] (BS3), (1,4-di-(3′-[2pyridyldithio]-propionamido)butane) (DPDPB), disuccinimidyl suberate (DSS), disuccinimidyl tartrate (DST), sulfodisuccinimidyl tartrate (Sulfo DST), dithiobis(succinimidyl propionate (DSP), 3,3′-dithiobis(sulfosuccinimidyl propionate (DTSSP), ethylene glycol bis(succinimidyl succinate) (EGS), bis(β-[4-azidosalicylamido]-ethyl)disulfide iodinatable (BASED), homobifunctional NHS crosslinking reagents (e.g., bis N-succinimidyl-[pentaethylene glycol] ester (Bis(NHS)PEO-5), and homobifunctional isothiocyanate derivatives of PEG or dextran polymers) and heterobifunctional cross-linking agents (e.g., succinimidyl 4-(N maleimidomethyl) cyclohexane-1-carboxylate (SMCC), succinimidyl-4-(N maleimidomethyl)-cyclohexane-1-carboxy(6-amidocaproate) (LC-SMCC), N maleimidobenzoyl-N-hydroxysuccinimide ester (MBS), succinimide 4-(p-maleimidophenyl) butyrate (SMPB), N-hydroxy-succinimide and N-ethyl-‘(dimethylaminopropyl)carbodiimide (NHS/EDC), (N-ε-maleimido-caproic acid)hydrazide (sulfoEMCS), N-succinimidyl-S-acetylthioacetate (SATA), monofluoro cyclooctyne (MFCO), bicyclo[6.1.0]nonyne (BCN), N-succinimidyl-S-acetylthiopropionate (SATP), maleimido and dibenzocyclooctyne ester (a DBCO ester), and 1-ethly-3-[3-dimethylaminopropyl]carbodiimide hydrochloride (EDC)).

By way of example, the nanostructures described herein, may be activated via an alkoxy silane (e.g., APTMS) to modify the free hydroxyl groups of the surface silanol groups to create a reactive group (for example, primary amines). The reactive group (for example, primary amines) created on the nanostructure then may be reacted with a cross-linking agent, for example, glutaraldehyde, that forms a covalent linkage with the free amine group present, for example, in the side chain of a lysine amino acid in a protein, for example, an antibody of interest.

It is contemplated that other activation and conjugation chemistries known in the art can be used to covalently couple one or more binding agents to the surface of the nanostructures described herein.

It is contemplated that a given nanostructure or series of nanostructures may be functionalized with a binding agent that binds an analyte of interest. The term “binding agent” as used herein refers to an agent that binds specifically to an analyte of interest. The terms “bind preferentially,” or “binds specifically” as used in connection with a binding agent refers to an agent that binds and/or associates (i) more stably, (ii) more rapidly, (iii) with stronger affinity, (iv) with greater duration, or (v) a combination of any two or more of (i)-(iv), with a particular target analyte than it does with a molecule other than the target analyte. For example, a binding agent that specifically or preferentially binds a target analyte is a binding domain that binds a target analyte, e.g., with stronger affinity, avidity, more readily, and/or with greater duration than it binds a different analyte. The binding agent may be an affinity for the analyte of about 100 nM, 50 nM, 20 nM, 15 nM, 10 nM, 9 nM, 8 nM, 7 nM, 6 nM, 5 nM, 4 nM, 3 nM, 2 nM, 1 nM, 0.5 nM, 0.1 nM, or 0.01 nM, or stronger, as determined by surface plasmon resonance. For example, the binding agent may have an affinity for the analyte within the range from about 0.01 nM to about 100 nM, from about 0.1 nM to about 100 nM, or from about 1 nM to about 100 nM. It is understood that a binding agent that binds preferentially to a first target analyte may or may not preferentially bind to a second target analyte. As such, “preferential binding” does not necessarily require (although it can include) exclusive binding.

Exemplary binding agents include enzymes (for example, that bind substrates and inhibitors), antibodies (e.g., that bind antigens), antigens (e.g., that bind target antibodies), receptors (e.g., that bind ligands), ligands (for example, that bind receptors), nucleic acid single-strand polymers (e.g., that bind nucleic acid molecules to form, e.g., DNA-DNA, RNA-RNA, or DNA-RNA double strands), and synthetic molecules that bind with target analytes. Natural, synthetic, semi-synthetic, and genetically-altered macromolecules may be employed as binding agents. Binding agents include biological binding agents, e.g., an antibody, an aptamer, a receptor, an enzyme, or a nucleic acid.

As used herein, unless otherwise indicated, the term “antibody” is understood to mean an intact antibody (e.g., an intact monoclonal antibody) or antigen-binding fragment of an antibody (for example, an antigen-binding fragment of a monoclonal antibody), including an intact antibody or antigen-binding fragment that has been modified, engineered, or chemically conjugated. Examples of antibodies that have been modified or engineered include chimeric antibodies, humanized antibodies, and multispecific antibodies (e.g., bispecific antibodies). Examples of antigen-binding fragments include Fab, Fab′, (Fab′)₂, Fv, single chain antibodies (e.g., scFv), minibodies, and diabodies.

In certain embodiments, an antibody binds to its target with a K_{D} of about 300 pM, 250 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, or 10 pM, or lower. An antibody may have a human IgG1, IgG2, IgG3, IgG4, or IgE isotype.

Methods for producing antibodies as well as other protein binding agents are known in the art. For example, the protein binding agents may be purified from natural sources or produced using recombinant DNA technologies. For example, DNA molecules encoding, for example, a protein binding agent can be synthesized chemically or by recombinant DNA methodologies. The resulting nucleic acids encoding desired protein-based binding agents can be incorporated (ligated) into expression vectors, which can be introduced into host cells through conventional transfection or transformation techniques. The transformed host cells can be grown under conditions that permit the host cells to express the genes that encode the proteins of interest. Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art. If the engineered gene is to be expressed in eukaryotic host cells, e.g., CHO cells, it is first inserted into an expression vector containing a suitable eukaryotic promoter, a secretion signal, a poly A sequence, and a stop codon. The gene construct can be introduced into eukaryotic host cells using conventional techniques. Thereafter, the host cells are cultured under conditions that permit expression of the protein based binding agent. Following expression, the polypeptide can be harvested and purified or isolated using techniques known in the art including, for example, affinity tags such as glutathione-S-transferase (GST) or histidine tags.

Exemplary nucleic acid based binding agents include aptamers and spiegelmers. Aptamers are nucleic acid-based sequences that have strong binding activity for a specific target molecule. Spiegelmers are similar to aptamers with regard to binding affinities and functionality but have a structure that prevents enzymatic degradation, which is achieved by using nuclease resistant L-oligonucleotides rather than naturally occurring, nuclease sensitive D-oligonucleotides.

Aptamers are specific nucleic acid sequences that bind to target molecules with high affinity and specificity and are identified by a method commonly known as Selective Evolution of Ligands by Evolution (SELEX), as described, for example, in U.S. Pat. Nos. 5,475,096 and 5,270,163. Each SELEX-identified nucleic acid ligand is a specific ligand of a given target compound or molecule. The SELEX process is based on the observation that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

The SELEX method applied to the application of high affinity binding involves selection from a mixture of candidate oligonucleotides and step-wise iterations of binding, partitioning and amplification, using the same general selection scheme, to achieve virtually any desired criterion of binding affinity and selectivity. Starting from a mixture of nucleic acids, preferably comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand enriched mixture of nucleic acids, then reiterating the steps of binding, partitioning, dissociating and amplifying through as many cycles as desired to yield highly specific high affinity nucleic acid ligands to the target molecule. Thus, this method allows for the screening of large random pools of nucleic acid molecules for a particular functionality, such as binding to a given target molecule.

The SELEX method also encompasses the identification of high-affinity nucleic acid ligands containing modified nucleotides conferring improved characteristics on the ligand, such as improved in vivo stability and protease resistance. Examples of such modifications include chemical substitutions at the ribose and/or phosphate and/or base positions. SELEX process-identified nucleic acid ligands containing modified nucleotides are described in U.S. Pat. Nos. 5,660,985 and 5,580,737, which include highly specific nucleic acid ligands containing one or more nucleotides modified at the 2′ position with, for example, a 2′-amino, 2′-fluoro, and/or 2′-O-methyl moiety.

Instead of using aptamers, which may require additional modifications to become more resistant to nuclease activity, it is contemplated that spiegelmers, mirror image aptamers composed of L-ribose or L-2′deoxyribose units (see, U.S. Pat. Nos. 8,841,431, 8,691,784, 8,367,629, 8,193,159 and 8,314,223) can be used in the practice of the invention. The chiral inversion in spiegelmers results in an improved plasma stability compared with natural D-oligonucleotide aptamers. L-nucleic acids are enantiomers of naturally occurring D-nucleic acids that are not very stable in aqueous solutions and in biological samples due to the widespread presence of nucleases. Naturally occurring nucleases, particularly nucleases from animal cells are not capable of degrading L-nucleic acids.

Using in vitro selection, an oligonucleotide that binds to the synthetic enantiomer of a target molecule, e.g., a D-peptide, can be selected. The resulting aptamer is then resynthesized in the L-configuration to create a spiegelmer (from the German “spiegel” for mirror) that binds the physiological target with the same affinity and specificity as the original aptamer to the mirror-image target. This approach has been used to synthesize spiegelmers that bind, for example, hepcidin (see, U.S. Pat. No. 8,841,431), MCP-1 (see, U.S. Pat. Nos. 8,691,784, 8,367,629 and 8,193,159) and SDF-1 (see, U.S. Pat. No. 8,314,223).

In an exemplary assay, one nanostructure array in one block of the well is functionalized with a binding agent (e.g., an antibody) that binds an analyte of interest. Each nanostructure array in each block of the well is functionalized with a different binding agent (e.g., an antibody). After functionalization, a sample (e.g., a plasma/serum sample) is added to the well under conditions to permit the binding agent to form a binding agent-analyte complex, if the analyte is present in the sample. The binding of analyte to the antibody results in a change in an optically detectable property of the nanostructure array, e.g., fluorescence.

A printing technique may be used to put different binding moieties, such as antibodies, on different nanostructures in a grid of nanostructure arrays disposed in a well. Printing may include, for example, contact printing (Gesim microcontact printer, Arrayit NanoPrint), inkjet printing (ArrayJet, Fujifilm), or piezo-electric dispensing (Perkin Elmer Piezorray, Biodot piezoelectric dispenser, Gesim NanoPlotter) of antibodies.

It is further contemplated that, for certain assays, for example, a label-free assay, formation of the binding agent-analyte complex (e.g., antibody 379-analyte 380 complex) alone results in a change in an optically detectable property of the nanostructure or array of nanostructures, e.g., nanoneedles 381 (FIG. 14C). For other assays, for example, label-based assays, the analyte in the sample can be universally attached with a functional group (for example, biotin). A second binding agent (for example, streptavidin, streptavidin-HRP or streptavidin-AP) can be used to further react to the functional group (e.g., biotin) that forms a complex with the analyte, which results in, or increases the change in, an optically detectable property of the nanostructure or array of nanostructures (e.g., a color change 382) (FIG. 14D). It is also contemplated that a third chemical reagent (for example, 3,3′,5,5′-Tetramethylbenzidine (TMB)) can have a chemical reaction with the second binding agent to form an additional substance on the nanostructure that further increases the change in an optically detectable property of the nanostructures, e.g., fluorescence.

In an exemplary sandwich immunoassay, one nanostructure array in one block of the well is functionalized with a binding agent (e.g., an antibody) that binds an analyte of interest. Each nanostructure array in each block of the well is functionalized with a different binding agent (e.g., an antibody). After functionalization, a sample (e.g., a plasma/serum sample) to be analyzed for the presence and/or amount of a target analyte is added to the well under conditions that permit the first binding agent to form a first binding agent-analyte complex, if the analyte is present in the sample. Then a second group of binding agents (e.g., a mix of secondary antibodies, e.g., a secondary antibody 383) that binds the analyte of interest is added to the nanostructure or series of nanostructures under conditions to permit the second binding agent to form a second binding agent-analyte complex. The binding of the analyte to the first and second binding agents results in a complex in a “sandwich” configuration. The formation of the sandwich complex can result in a change in an optically detectable property of the nanostructure or arrays of nanostructures (e.g., a color change 382). It is also contemplated that the second antibody can be labeled with a functional group (e.g., biotin), thus a third binding agent (e.g., streptavidin) can be further attached to the second binding agent to form additional substance on the nanostructure that further increase the change in an optically detectable property of the nanostructures (FIG. 14E).

The binding agent can be monoclonal antibodies, polyclonal antibodies, recombinant antibodies, nanobodies, fractions of antibodies and etc. The binding agent can also be aptamers. Aptamers are specific nucleic acid sequences that bind to target molecules with high affinity and specificity and are identified by a method commonly known as Selective Evolution of Ligands by Evolution (SELEX), as described, for example, in U.S. Pat. Nos. 5,475,096 and 5,270,163. Each SELEX-identified nucleic acid ligand is a specific ligand of a given target compound or molecule. The SELEX process is based on the observation that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size or composition can serve as targets.

The nanostructures can be functionalized using standard chemistries known in the art. As an initial matter, the surfaces of the nanostructures may be activated for binding a binding agent using standard chemistries, including standard linker chemistries.

The binding agent may contain or be engineered to contain a functional group capable of reacting with the surface of the nanostructure (e.g., via silanol groups present on or at the surface of the nanostructure), either directly or via a chemical linker.

In one approach, the surface silanol groups of the nanostructure may be activated with one or more activating agents, such as an alkoxy silane, a chlorosilane, or an alternative silane modality, having a reactive group (e.g., a primary amine). Exemplary alkoxy silanes having a reactive group may include, for example, an aminosilane (e.g., (3-aminopropyl)-trimethoxysilane (APTMS), (3-aminopropyl)-triethoxysilane (APTES), (3-aminopropyl)-diethoxy-methylsilane (APDEMS), 3-(2-aminoethylaminopropyl)trimethoxysilane (AEAPTM)), a glycidoxysilane (e.g., (3-glycidoxypropyl)-dimethyl-ethoxysilane (GPMES)), or a mercaptosilane (e.g., (3-mercaptopropyl)-trimethoxysilane (MPTMS) or (3-mercaptopropyl)-methyl-dimethoxysilane (MPDMS). Exemplary chlorosilanes having a reactive group include 3-(trichlorosilyl)propyl methacrylate (TPM) and 10-isocyanatodecyltrichlorosilane.

Thereafter, a functional group on the binding agent, for example, a primary amine on the side chain on a lysine residue can be attached to the reactive group added to the surface of the nanostructure using a variety of cross-linking agents. Exemplary cross-linking agents can include, for example, homobifunctional cross-linking agents (e.g., glutaraldehyde, bismaleimidohexane, bis(2-[Succinimidooxycarbonyloxy]ethyl) sulfone (BSOCOES), [bis(sulfosuccinimidyl)suberate] (BS3), (1,4-di-(3′-[2pyridyldithio]-propionamido)butane) (DPDPB), disuccinimidyl suberate (DSS), disuccinimidyl tartrate (DST), sulfodisuccinimidyl tartrate (Sulfo DST), dithiobis(succinimidyl propionate (DSP), 3,3′-dithiobis(sulfosuccinimidyl propionate (DTSSP), ethylene glycol bis(succinimidyl succinate) (EGS), bis(β-[4-azidosalicylamido]-ethyl)disulfide iodinatable (BASED), homobifunctional NHS crosslinking reagents e.g., bis N-succinimidyl-[pentaethylene glycol] ester (Bis(NHS)PEO-5), and homobifunctional isothiocyanate derivatives of PEG or dextran polymers) and heterobifunctional cross-linking agents (e.g., succinimidyl 4-(N maleimidomethyl) cyclohexane-1-carboxylate (SMCC), succinimidyl-4-(N maleimidomethyl)-cyclohexane-1-carboxy(6-amidocaproate) (LC-SMCC), N maleimidobenzoyl-N-hydroxysuccinimide ester (IBS), succinimide 4-(p-maleimidophenyl) butyrate (SMPB), N-hydroxy-succinimide and N-ethyl-‘(dimethylaminopropyl)carbodiimide (NHS/EDC), (N-ε-maleimido-caproic acid)hydrazide (sulfoEMCS), N-succinimidyl-S-acetylthioacetate (SATA), monofluoro cyclooctyne (MFCO), bicyclo[6.1.0]nonyne (BCN), N-succinimidyl-S-acetylthiopropionate (SATP), maleimido and dibenzocyclooctyne ester (a DBCO ester), and 1-ethyl-3-[3-dimethylaminopropyl]carbodiimide hydrochloride (EDC)).

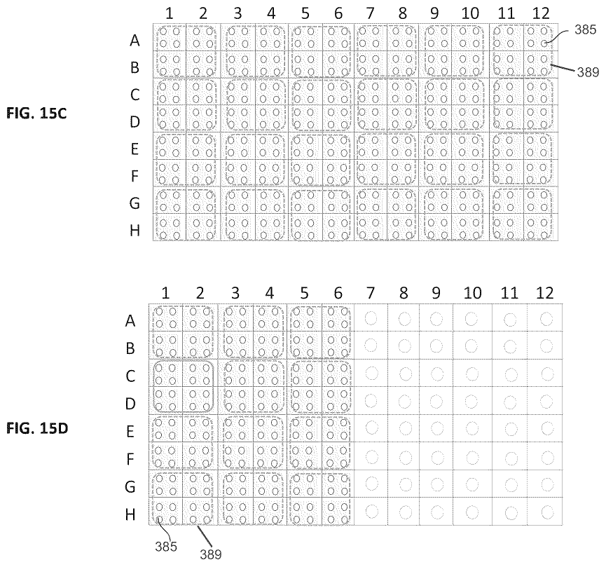

In some embodiments, a customizable gasket based approach can be used to mask areas of the chip and, e.g., antibodies, aptamers, or other binding reagents can be functionalized at designated positions on the chip. For example, FIG. 15A shows a 4-plex gasket 385 that matches with the SBS 96 plate layout. The gasket has four small wells 386 inside the dimension of the SBS 96 single well 387. Solutions can be either hand-pipetted or spotted with liquid handlers into each well. The number of the small wells can be different across the entire 96-well plate. In a modification of this embodiment, see FIG. 15B, where half of the plate has 16 small wells (16-plex 388) within the SBS 96 single well, and the other half of the plate has only one well inside the SBS 96 single well. It is contemplated herein that a fraction of a plate (e.g., ¼ or ½ the plate) may contain a gasket of one size, and the other fraction or fractions may contain a gasket of another size, or no gasket.

In some embodiments, the gasket is made using vinyl cutting for coarse dimensions. In certain embodiments, said coarse dimensions are at or above about 1 mm. In some embodiments, laser cutting can be used to achieve a feature size at or above about 25 μm. In some embodiments, soft-lithography patterning can be used to achieve at or above about 0.5 μm feature sizes. In some embodiments, soft-lithography patterning can be used to achieve at or above about 0.5 μm feature sizes.

In some embodiments, samples are loaded onto the chip, and different groups of wells are covered under a second gasket layer. Such an embodiment is shown in FIG. 15C, where the first layer gasket has four small wells inside a single SBS 96 well. Different binding reagents are functionalized on the surface of each of the small wells separately. Next, a second gasket layer that covers four of the SBS 96 well (thus, covering 16 small wells) is made to mask the surface of the chip. Finally, samples are loaded into the large wells 389 (indicated in broken lines in FIG. 15C). Similar to the first layer, wells in the second layer do not need to be the same dimensions. An example of this embodiment is shown in FIG. 15D, where the wells on the left side half of the second gasket layer have a dimension that covers four of the SBS 96 single wells, and the wells on the right half cover only one SBS 96 single well.

### II. Cartridge

The sensors described herein, once fabricated, can be included in, or otherwise assembled into, a cartridge for use within a detection system. The cartridge may be used for detecting the presence, or quantifying the amount, of an analyte in a sample of interest. The cartridge comprises a housing defining at least one well comprising any one or more of the foregoing sensors. The housing may define a plurality of wells, each well comprising any one or more of the foregoing sensors. The wells can be defined by (e.g., integral with) the substrate or can be defined by a hole formed in a gasket disposed upon the substrate.

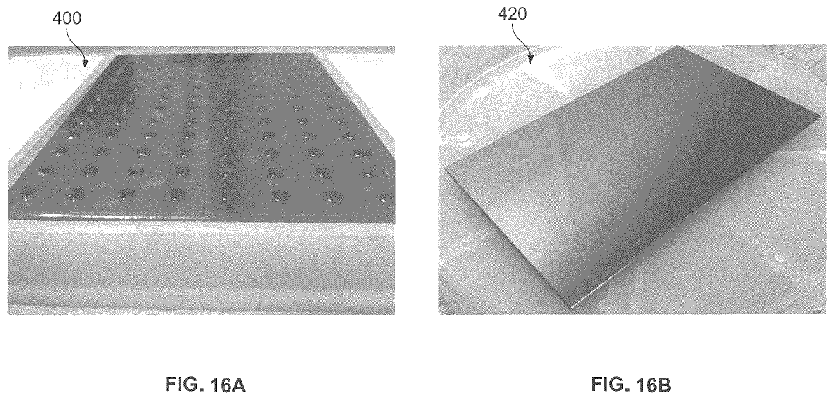

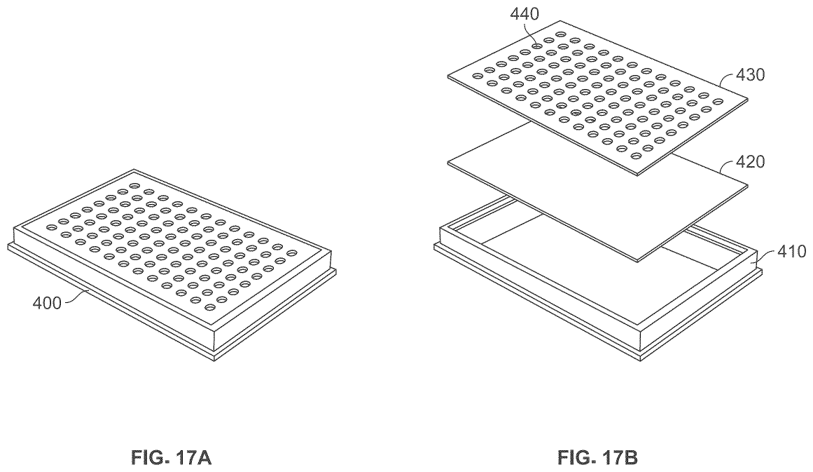

Referring to FIGS. 16A, 16B, 17A and 17B, the sensors described herein may be incorporated into a cartridge assembly (a consumable assembly) 400. The cartridge assembly may include a housing or base 410, a wafer substrate 420 upon which the series of nanostructures are disposed, and gasket 430. The gasket 430, when placed over wafer substrate 420, can define wells, wherein the base of each well can comprise one or more sensors. The wafer substrate interfits into housing or base 410, which is configured to hold the substrate and to be easily insertable into a detection system. The housing or base may be made from a variety of different materials, for example, a metal such as aluminum, as well as plastic or rubber. The housing or base may have a feature, such as an angled corner, to facilitate placement thereof into the sensor system and/or to confirm orientation.

Gasket 430 can be fabricated, for example, from silicone or plastic, sized and shaped to be placed over the wafer substrate, with openings 440 dimensioned to create wells with the wafer substrate containing the sensors disposed upon or within the wafer substrate. The openings 440 that define the wells may be dimensioned to contain at least a portion of the sample, for example, 2, 3, 4, 5, 6, 7, 8, 9, 10, 20, or 50 μL, to be analyzed. Typically, a well includes walls defined by the gasket 430 and a bottom portion defined by the wafer substrate 420, with a sensor being disposed on the substrate in the well. A diameter of the well may range from 600 μm to 90 mm (e.g., from 1 mm to 80 mm) and may have a thickness of 1 mm. In some embodiments, the wells may be formed integrally with the substrate during the fabrication process.

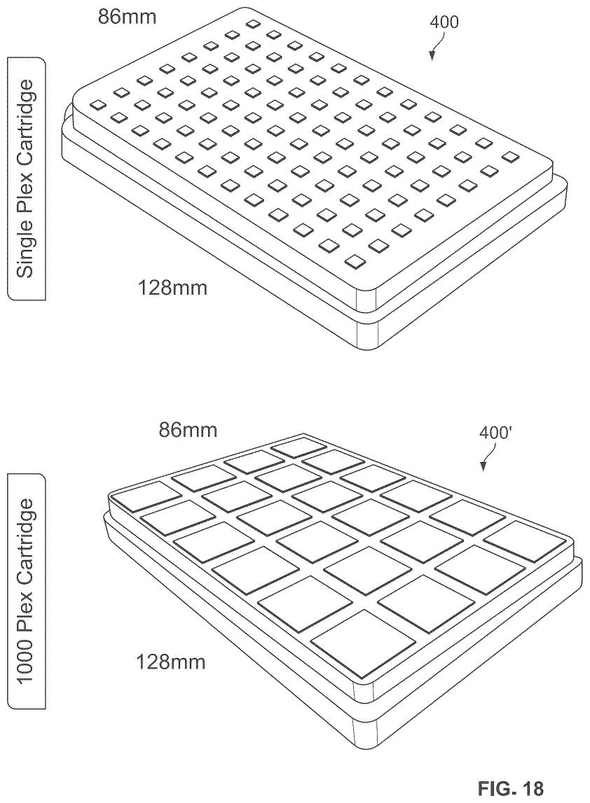

FIG. 18 shows a perspective view of a single plex consumable cartridge 400 and a 1,000 plex consumable cartridge 400′. In these embodiments, the sensor for the single plex cartridge is configured to detect and/or quantify a single analyte, whereas the 1,000 plex cartridge is configured to simultaneously detect and/or quantify up to 1,000 different analytes. Also, the dimensions and placement of wells 440 in the gasket 430 is adjusted to accommodate the number of sensors to be included in a single well. It is understood that the technologies described herein are scalable and the cartridge may be fabricated in a wide range of shapes and sizes. In certain embodiments, the cartridge is configured to meet Society for Biomolecular Screening (SBS) dimensional standards for microplates, for example, standard 96 well microplates. Accordingly, both the wafer substrate and the base may be rectangular in shape, with the base having a length of 128 mm and a width of 86 mm, which facilitates interfacing with various liquid handling systems and ease of portability on various liquid handling platforms.

### III. System Considerations

Also described herein is a system for detecting the presence, or quantifying the amount, of an analyte in a sample of interest. The system comprises (a) a receiving chamber for receiving any one or more of the foregoing sensors any one or more of the foregoing cartridges; (b) a light source for illuminating at least the first series and/or any second series and/or any third series of nanostructures; and (c) a detector for detecting a change in an optical property in at least the first series and/or any second series and/or any third series of nanostructures; and optionally (d) a computer processor implementing a computer algorithm that identifies an interface between the first concentration range and optionally any second concentration range and optionally an interface between any second concentration range and any third concentration range.

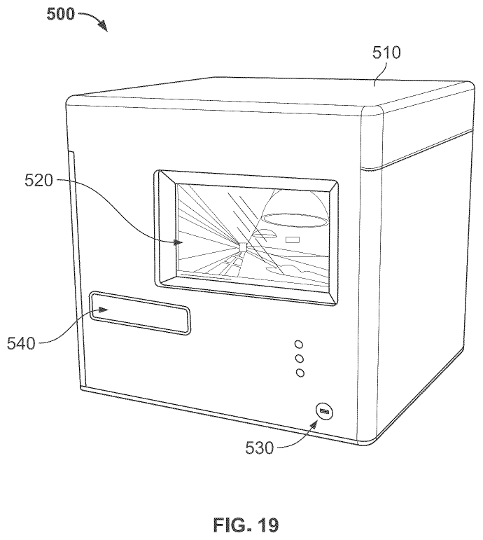

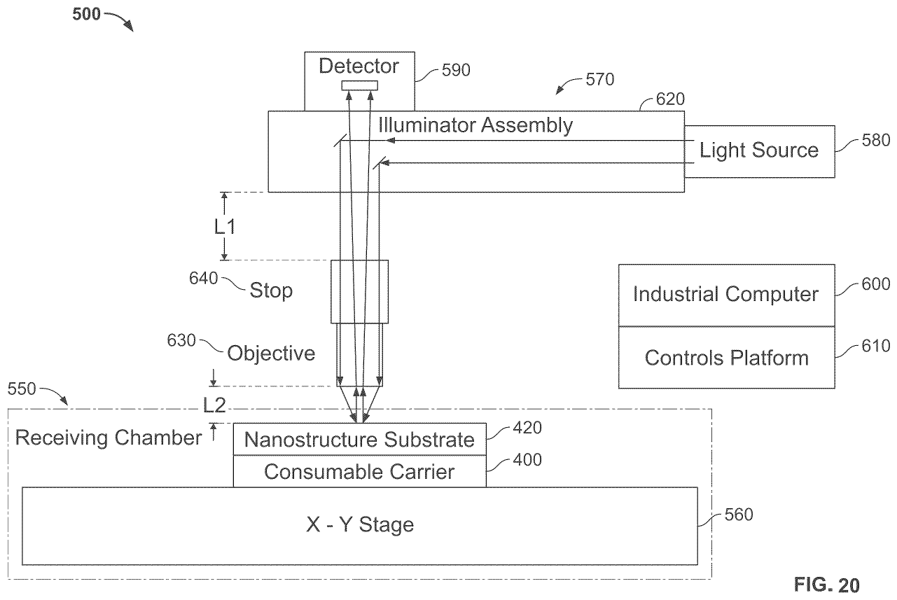

With reference to FIGS. 19 and 20, an exemplary sensor system 500 is configured to facilitate the detection, or quantification of the amount, of an analyte in a sample of interest. The sensor system 500 can include a system housing 510 with a touch screen interface 520 and, for example, a data port 530. A load/unload door 540 in the housing may be sized and configured to enable the introduction of a cartridge 400 into a receiving chamber 550 of the sensor system that contains, for example, an X-Y stage 560 for holding and positioning the cartridge relative to an optical detection system 570. A light source 580 is configured to transmit a light through a camera/detector 590. The camera is configured to be positioned over the cartridge during use, and to detect a change in an optical property in at least a first, a second, and/or a third series of nanostructures on the substrate 420 disposed in the cartridge. The light source 580 is configured to illuminate nanostructures, for example, nanostructures disposed on the wafer substrate of a cartridge. The system can include a computer 600 including a computer processor for implementing the algorithm for identifying an interface between first concentration ranges and/or second concentration ranges and/or third concentration ranges, and for quantifying analytes in samples. The sensor system may also include a control platform 610 for controlling the system. Accordingly, the system includes three major sub-assemblies: a control system, an imaging system, and a cartridge handling system. These sub-assemblies may employ commercially available components to minimize supply chain complexity and to reduce assembly time.

The imaging system includes the optical detection system 570, in which the light source 580 is configured to direct light through an illuminator assembly 620 and an objective 630 to impinge on a plurality of nanostructures disposed upon a substrate of the sensor. After interacting with the sensor, the reflected light passes through the objective 630 and is captured by the detector 590. A stop 640 is disposed above the objective 630. The stop is a dark field light stop, which controls illumination, including how illumination reaches the substrate and how the image is transmitted to the detector. The mechanical tube length of the microscope system is indicated as L1, and may range from 10 mm to 300 mm. A working distance of the objective is designated as L2, and may range from about 2 mm to about 5 mm. In certain embodiments, L1 is greater than L2.

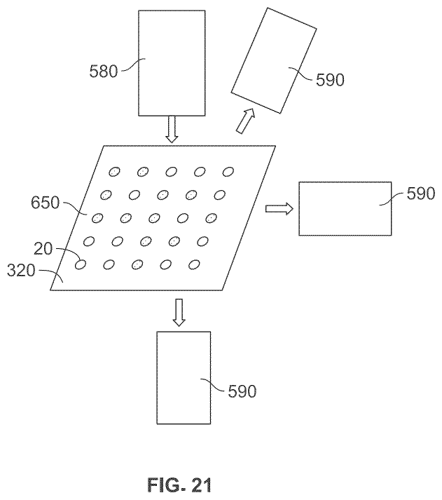

As illustrated in FIG. 21, the measurement can be an optical measurement. For example, light source 580 can be used to irradiate substrate 320 with nanostructures 20 and analytes 650 disposed thereon, and one or more detectors 590 is/are positioned to detect the light that impinges the substrate. The light that is deflected from the substrate can be in the same direction of the light source, in the opposite direction, at orthogonal direction or at an angle to the light source. The data present in the images obtained by use of the optical detection system can be processed to provide the concentration of analyte present in a sample.

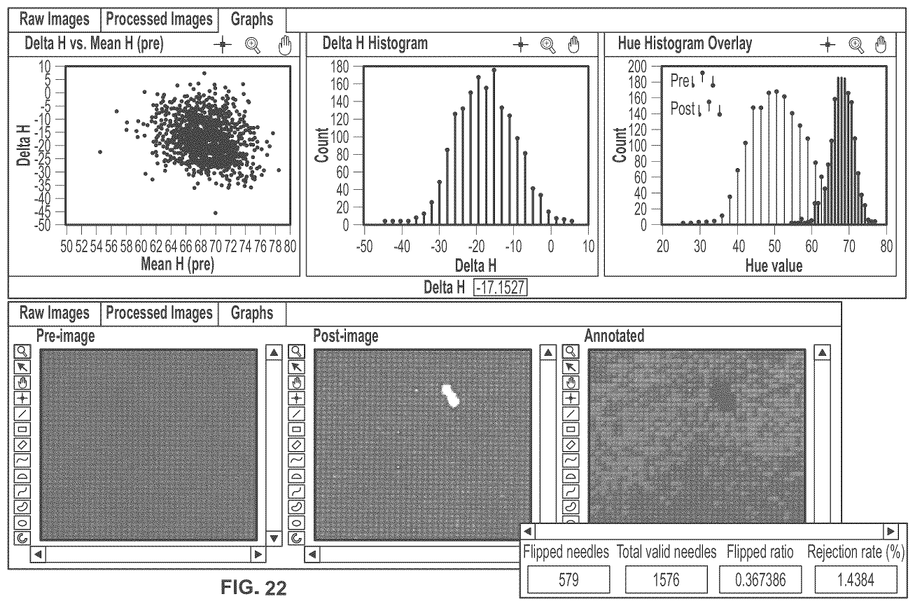

FIG. 22 shows one approach to informatics related to various embodiments of the sensor and related system. On average, all of the nanostructures in a given region are of substantially the same configuration and statistically have a substantially similar quantity or number of analyte binding sites. Accordingly, for a given concentration of analyte in the sample, each nanostructure in that region can be expected to bind the same number of molecules. In order for the sensor to have a wide dynamic range, a plurality of digital and analog regions with nanostructures of various configurations can be provided.

As the concentration of analyte in the samples range from the lowest detectable concentration to the highest detectable concentration in the digital regions of the sensor, the system is configured to detect the quantity or number of nanostructures evidencing an isolated color change corresponding to the binding of analyte above a threshold value (e.g., by flipping from one state to another). The higher the percentage of discrete nanostructures that exhibit a detectable color change or that have flipped, the higher the number of bound analytes and, accordingly, the higher the concentration of analyte in the sample. As depicted in FIG. 22, this flipping behavior can be presented visually in a variety of formats, including scatter plots that show data clustering, histograms that show data distribution, etc. Comparative images of each region can also be provided, showing a particular region of the sensor before exposure to the sample, as well as after exposure. A third annotated image can be provided depicting with greater clarity the results of the flipping determination. Numerical data is also advantageously presented, indicating absolute numbers of flipped and valid nanostructures, as well as the associated ratio value of the flipped to valid nanostructures. In particular, “flipped needles” denotes the number of sensors that have exceeded the threshold and are counted as positive. “Total valid needles” denotes the number of sensors that are counted as part of the total population. Sensors that behave outside of expected parameters are discarded and not included in subsequent analysis. Only the sensors that remain are considered “valid”. The flipped ratio is the calculated value of flipped needles divided by total valid needles. The rejection rate can also be depicted, i.e., the percentage of needles that are discarded from the pre-image. This is used as a measure of sensor quality/health. Sensors with rejection rate values of around 10% or higher are considered poor quality and generally do not provide reliable data.

At some higher threshold concentration, however, all of the digital region nanostructures have bound analyte. The digital regions of the sensor have effectively become saturated. All nanostructures have flipped and no local color change is readily evident. At this point, attention is shifted to the analog regions, that generally have larger nanostructures with more numerous binding sites.

The degree of color change of a given nanostructure can be related to the ratio of the total mass of bound molecules to the total mass of that nanostructure. Smaller analog region nanostructures (e.g., nanoneedles) that may only be able to bind less than 100 molecules can evidence a cool color hue initially (e.g., in the blue/green range). Larger analog region nanostructures (e.g., nanoneedles) that may be able to bind a few hundred molecules can evidence a warmer color hue initially (e.g., in the yellow/orange range). At the higher detectable concentrations in the analog regions, as more analytes bind to a given nanostructure, the detectable color hue shifts more warmly. Accordingly, an unexposed blue nanostructure exhibits a more greenish hue after binding for a particular analyte concentration in the sample. At higher analyte concentrations in the sample, the hue can shift to be more yellowish. Similarly, in an analog region with larger nanostructures and more binding sites configured to detect higher concentrations, the initial unexposed yellow nanostructure exhibits a more orange hue after binding for a particular analyte concentration in the sample. At higher analyte concentrations in the sample, the hue can shift to be more reddish.

While the color shift is detectable with solely a single analog nanostructure, regions of a series or array of similarly sized nanostructures are advantageously employed. By providing a large distribution of similarly sized nanostructures, an average readout can be provided to more reliably detect the analog region color shift and, accordingly, the detected analyte concentration.

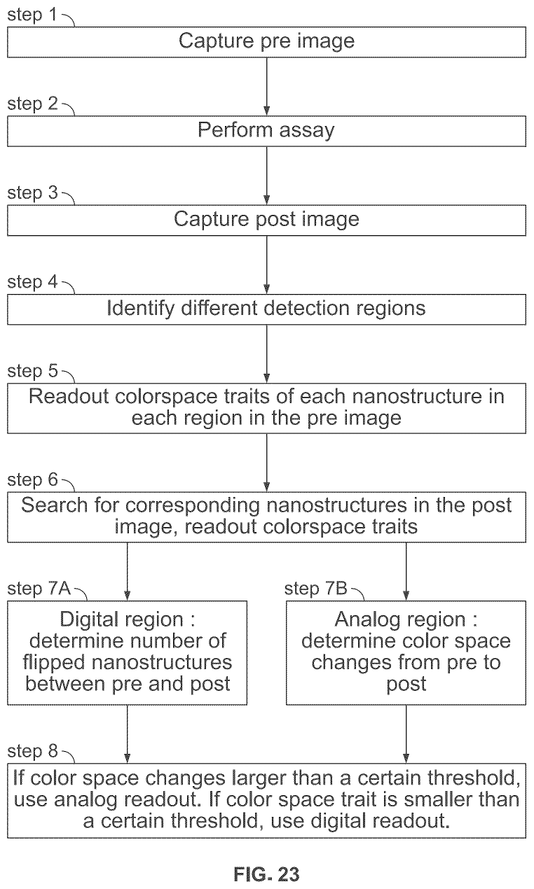

More specifically, FIG. 23 shows a flowchart of one approach for aggregating, at a system level, the detected output of the various digital and analog regions of one embodiment of a sensor, to reliably detect analyte concentration across the full dynamic range of the sensor. Use of this form of hybrid informatic engine algorithm permits the use of discrete digital and analog regions to reliably reject inaccurate higher concentration data from the digital regions and inaccurate lower concentration data from the analog regions.

In Step 1 of FIG. 23, the various digital and analog regions of a clean sensor are optically imaged as part of an overall image of the sensor, to provide a reliable baseline recording of the image status of each region and its associated nanostructures (e.g., presence or absence, initial color hue, etc.) for a particular sensor. In Step 2, the sensor is exposed to the sample, any analytes in the sample bind to associated sites on the nanostructures, and the sensor is subsequently conventionally prepared for subsequent imaging. In Step 3, the system captures the post exposure image of the sensor, that will be used to compare to the image of Step 1 to detect flipping in the digital regions and any color hue change in the analog regions. In Step 4, the algorithm identifies the different detection regions of the sensor (i.e., one or more digital regions and one or more analog regions) and their layout relative to the fiducial mark of the sensor. This permits the system to correlate and align the pre and post images to identify corresponding nanostructures in each image. Steps 5 and 6 entail individual, discrete analysis of the pre and post image data on a nanostructure-by-nanostructure basis in each corresponding region. For digital regions, Step 7A quantifies and counts the number of nanostructures with bound analyte by confirming a sufficiently large shift in the local image above a threshold to identify each nanostructure that has bound analyte. For analog regions, Step 7B detects color hue changes locally and across the analog region, evidencing a sufficiently large shift in the local image above the pre image color to deem the nanostructures locally and collectively to have bound analyte. In Step 8, assuming the color change in the analog region exceeds a predetermined threshold value, the analog region is deemed to have detected a concentration of analyte within its detectable range. The actual concentration of analyte corresponding to the color change is determined by comparison of the detected color change to a standard curve stored in system memory developed with known concentration control samples. If, however, the color change in the analog region fails to exceed a predetermined threshold value, the concentration of analyte is deemed to be below that reliably detectable by that analog region. If a lower concentration-configured analog region is available, a similar analysis can be performed. Otherwise, the system relies on the digital count of flipped nanostructures in the digital regions of the sensor. The actual concentration of analyte corresponding to the quantity or number of flipped nanostructures is determined by comparison of the number of flipped digital nanostructures to a standard curve stored in system memory developed with known concentration control samples.

In another embodiment, an exemplary algorithm for determining the transition between a digital quantification measurement and an analog comprises the steps of (a) measuring the nanostructures that have changed (flipped) from one state to another relative to the nanostructures in the first series upon application of the solution to be tested; (b) measuring the color space changes of nanostructures in the second series upon application of the solution to be tested; and (c) if the color space change of the second series is greater than a preselected threshold value then use the analog measurements identified in step (b) and if the color space changes of the second series is less than the preselected threshold value, then use the digital measurements identified in step (a).

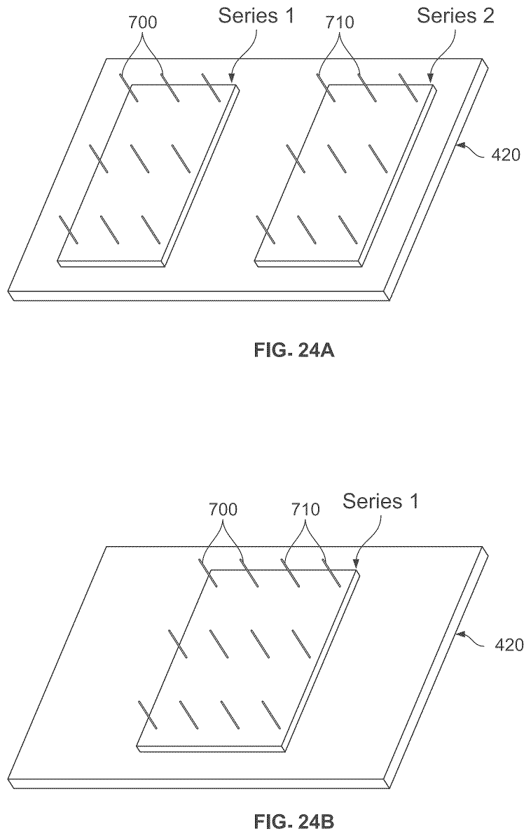

It is contemplated that, based on the choice of nanostructure (e.g., nanoneedle) and binding agent and other reagents, it is possible to detect and/or quantify multiple analytes at the same time. For example, as shown in FIG. 24A, a sensor can comprise a substrate 420 having disposed thereon a first series of nanostructures 700 and a second series of nanostructures 710 that can bind two separate and distinct analytes. It is contemplated that the substrate can contain a number of series of nanostructures, depending upon the number of analytes to be detected. Similarly, as shown in FIG. 24B, a sensor can comprise a substrate having disposed thereon a series of two different nanostructures 700, 710 that bind two separate and distinct analytes. It is contemplated that the series of nanostructures can contain nanostructures that bind to additional analytes.

### IV. Assays

Also described herein is a method of detecting the presence, or quantifying the amount, of an analyte, e.g., a protein, in a sample of interest. The method comprises: (a) applying at least a portion of the sample to any one or more of the foregoing sensors; and (b) detecting a change in an optical property of the first series and/or any second series and/or any third series of nanostructures thereby to detect the presence, or quantify the amount, of the analyte in the sample.

The sensor may detect the analyte is a variety of samples, for example, a body fluid, a tissue extract, and/or a cell supernatant. Exemplary body fluids include, for example, blood, serum, plasma, urine, cerebrospinal fluid, or interstitial fluid.

The method comprises combining at least a portion of a sample with a structure, sensor, cartridge, or system described herein, and detecting the presence and/or quantifying the amount of binding of the analyte to the structure, sensor, cartridge, or system. For example, following binding of an analyte to a nanostructure or a series of nanostructures described herein, the binding of the analyte may be detected by a change in an optically detectable property of the nanostructure or series of nanostructures. In certain embodiments, the optically detectable property is color, light scattering, refraction, or resonance (for example, surface plasmon resonance, electric resonance, electromagnetic resonance, and magnetic resonance). In certain embodiments, electromagnetic radiation may be applied to the nanostructure or a series of nanostructures, and the applied electromagnetic radiation may be altered as the nanostructure or series of nanostructures interacts with the sample suspected of containing an analyte. For example, the presence of the analyte may result in a change of intensity, color, or fluorescence.

In another embodiment, the method includes applying a portion of the sample to a sensor comprising a first region and a second region. The first region comprises a first series of nanostructures capable of binding the analyte and producing a detectable signal indicative of a concentration of the analyte in the sample within a first concentration range. The second region comprises a second series of different nanostructures capable of binding the analyte and producing a detectable signal indicative of a concentration of the analyte in the sample within a second, different concentration range. The regions are interrogated, for example, using electromagnetic radiation to detect detectable signals from the first and second series of nanostructures, the signals being indicative of the presence and/or amount of analyte in the sample. The presence and/or amount of the analyte can then be determined from the detectable signals thereby to detect the presence, or to quantify the amount of, the analyte in the sample across both the first concentration range and the second concentration range.

In another embodiment, the method includes applying a portion of the sample to a sensor comprising a first region and a second region. The first region comprises a first series of nanostructures capable of binding the analyte and producing a detectable signal indicative of a concentration of the analyte in the sample within a first concentration range, wherein individual nanostructures of the first series that bind the analyte are optically detected upon binding the analyte, whereupon the concentration of analyte in the sample, if within the first concentration range, is determined from a number of individual nanostructures in the first series that have bound molecules of analyte. The second region comprises a second series of different nanostructures capable of binding the analyte and producing a detectable signal indicative of a concentration of the analyte in the sample within a second, different concentration range, wherein the concentration of analyte in the sample, if within the second concentration range, is determined by analog detection of a substantially uniform change in an optically detectable property of the nanostructures in the second region as a function of the concentration of the analyte. The regions are interrogated, for example, using electromagnetic radiation to detect detectable signals from the first and second series of nanostructures, the signals being indicative of the presence and/or amount of analyte in the sample. The presence and/or amount of the analyte can then be determined from the detectable signals thereby to detect the presence, or to quantify the amount of, the analyte in the sample across both the first concentration range and the second concentration range.

In an exemplary assay, a nanostructure or series of nanostructures is functionalized with a binding agent (e.g., an antibody) that binds an analyte of interest. After functionalization, a sample (e.g., a fluid sample) including the target analyte is added to the nanostructure or series of nanostructures under conditions to permit the binding agent to form a binding agent-analyte complex, if the analyte is present in the sample. The binding of analyte to the antibody results in a change in an optically detectable property of the nanostructure or series of nanostructures. It is contemplated that, for certain assays, for example, a label free assay, formation of the binding agent-analyte complex alone results in a change in an optically detectable property of the nanostructure or series of nanostructures. For other assays, for example, label-based assays, the second binding agent that forms a complex with the analyte may also include a label that directly or indirectly in the complex results in, or increases the change in, an optically detectable property of the nanostructure or series of nanostructures. It is contemplated that nanostructures can detect the presence and/or amount of an analyte without having a particle or bead attached to or otherwise associated with the nanostructure.

In an exemplary sandwich immunoassay, a nanostructure or series of nanostructures is functionalized with a first binding agent (e.g., a first antibody) that binds the analyte of interest. After functionalization, a sample (e.g., a fluid sample) to be analyzed for the presence and/or amount of a target analyte is added to the nanostructure or series of nanostructures under conditions that permit the first binding agent to form a first binding agent-analyte complex, if the analyte is present in the sample. Then a second binding agent (e.g., a second antibody) that binds the analyte of interest is added to the nanostructure or series of nanostructures under conditions to permit the second binding agent to form a second binding agent-analyte complex. The binding of the analyte to the first and second binding agents results in a complex in a “sandwich” configuration. The formation of the sandwich complex can result in a change in an optically detectable property of the nanostructure or series of nanostructures. It is contemplated, however, that for certain assays for example, label-free assays, formation of the sandwich complex alone results in a change in an optically detectable property of the nanostructure or series of nanostructures. For other assays, for example, label-based assays, the second binding agent in the sandwich complex can include a label that either directly or indirectly results in or increases the change in an optically detectable property of the nanostructure or series of nanostructures.

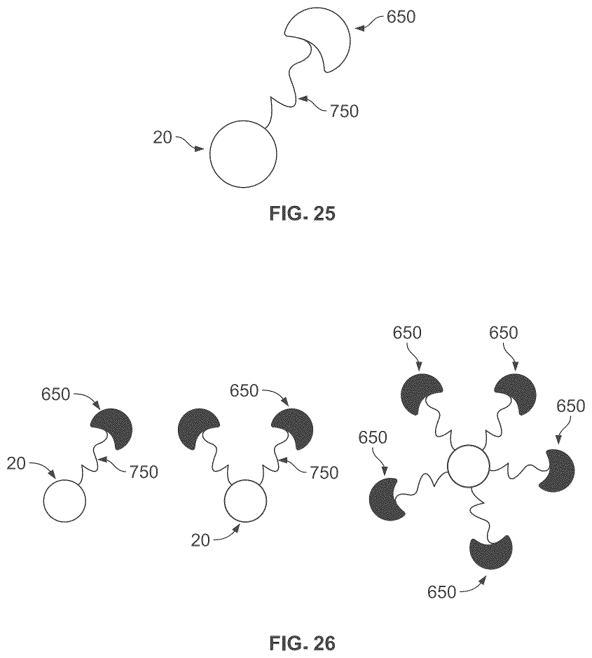

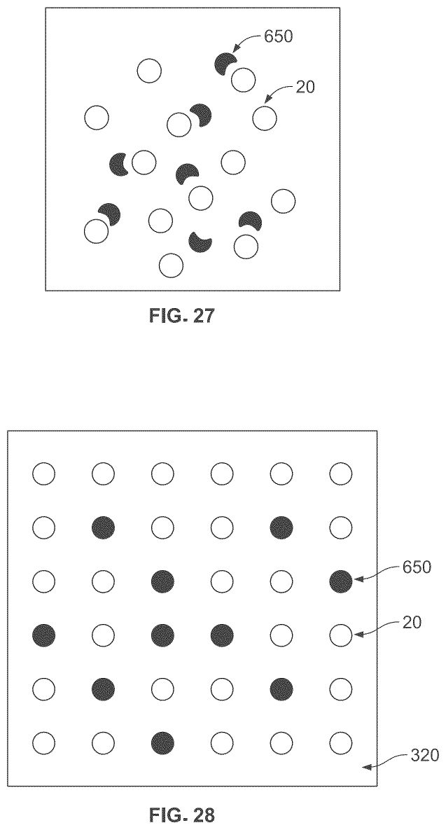

FIG. 25 depicts an exemplary assay whereby an analyte 650 interacts with a binding agent 750 immobilized on a nanostructure 20. The capturing capacity of the nanostructure is determined by both the dimensional relation between the nanostructure and the available capturing agent. FIG. 26 depicts an exemplary assay where there is a 1:1 ratio between nanostructure 20 and bound analyte 650 (left panel), a 1:2 ratio between nanostructure and bound analyte (center panel), and a 1:5 ratio between nanostructure and bound analyte (right panel). FIG. 27 depicts an exemplary assay where nanostructures 20 outnumber analytes 650, in which case, each nanostructure is likely to capture at most one analyte. The nanostructures 20 can be directly fabricated with nanofabrication technologies on a substrate, as discussed above. FIG. 28 depicts nanofabricated nanostructures 20 disposed on a silicon substrate 320, with analytes 650 bound to a portion of the nanostructures. The binding between analytes and nanostructures occur on a solid interface. The nanostructures may be measured to determine the number of binding analytes on its surface. FIGS. 25-28 depict examples of a label-free immunoassay wherein a single binding agent (e.g., antibody or aptamer) is used to bind a target analyte. This method can be used to measure or otherwise quantify binding affinities, binding kinetics (on and off rate), etc.

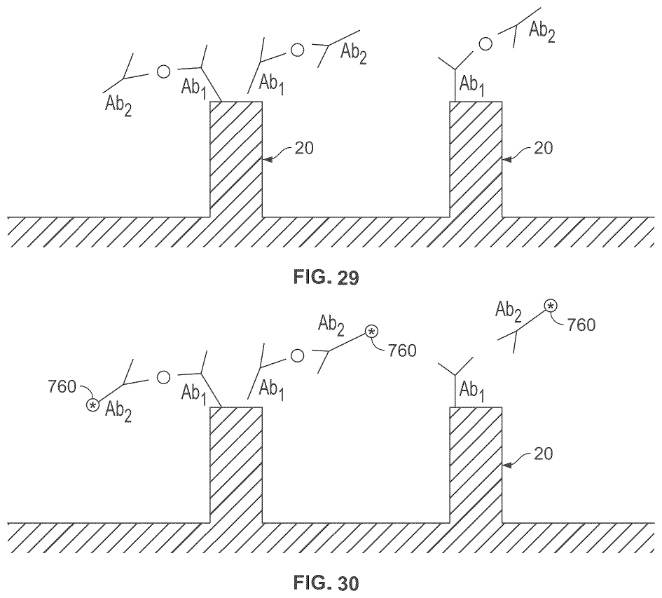

FIG. 29 depicts an exemplary label-free immunoassay wherein a plurality of first antibodies (Ab1) are immobilized upon the fluid exposed surface of a nanostructure 20. Thereafter, a sample including the analyte to be detected and/or quantified (0) is contacted with the nanostructures either alone or in combination with a second antibody (Ab1) that binds the analyte, preferably via a second, different epitope. The second antibody (Ab2) can be added after the analyte. The two antibodies (Ab1 and Ab2) and analyte (0), if present, form a complex that is immobilized on the surface of the nanostructure 20. The binding of the complex to the nanostructure may cause a change in a property of the nanostructures that can be detected with a detection system. FIG. 30 depicts an exemplary label-based immunoassay that is performed essentially as described above in connection with FIG. 29, except that, in this embodiment, the second antibody is labeled. The binding of the complex to the nanostructure 20 can be detected via the label 760, either directly (for example, via a gold label) or indirectly (for example, via an enzyme that creates a further product) to cause a change in a property of the nanostructures that can be detected with the detection system.

In an alternative assay, a sample (e.g., a fluid sample) to be analyzed for the presence and/or amount of a target analyte is incubated with (i) a first binding agent (e.g., an antibody) under conditions to permit the first binding agent to form a first binding agent-analyte complex, if the analyte is present in the sample, and (ii) a second binding agent (e.g., a second antibody) that binds the analyte of interest under conditions to permit the second binding agent to form a second binding agent-analyte complex. The binding of the analyte to the first and second binding agents results in a complex in a “sandwich” configuration, which occurs free in solution. Then, depending upon the assay, the first binding agent, second binding agent, and/or analyte, either complexed or uncomplexed, are added to a nanostructure or series of nanostructures, under conditions such that the complex or component thereof is bound by the nanostructure or series of nanostructures to create a change in a property (e.g., an optically detectable property) of the nanostructure or series of nanostructures. In certain embodiments, one or both of the antibodies is labeled with biotin, and the sandwich complex can become immobilized on the surface if any nanostructure or a series of nanostructures that have been functionalized with, for example, avidin or biotin.

Typically, when the binding agent is an antibody, then between each assay step, the nanostructure with bound analyte can be washed with a mild detergent solution. Typical protocols also include one or more blocking steps, which involve use of a non-specifically-binding protein such as bovine serum albumin or casein to block or reduce undesirable non-specific binding of protein reagents to the nanostructure.

Exemplary labels for use in label-based assays include a radiolabel, a fluorescent label, a visual label, an enzyme label, or other conventional detectable labels useful in diagnostic or prognostic assays, for example, particles, such as latex or gold particles, or such as latex or gold sol particles. Exemplary enzymatic labels include, for example, horseradish peroxidase (HRP), alkaline phosphatase (AP), β-galactosidase (β-Gal), and glucose oxidase (GO). When the label is an enzyme, the assay includes the addition of an appropriate enzyme substrate that produces a signal that results in a change in an optically detectable property of the nanostructure or series of nanostructures. The substrate can be, for example, a chromogenic substrate or a fluorogenic substrate. Exemplary substrates for HRP include OPD (o-phenylenediamine dihydrochloride; which turns amber after reaction with HRP), TMB (3,3′,5,5′-tetramethylbenzidine; which turns blue after reaction with HRP), ABTS (2,2′-azino-bis [3-ethylbenzothiazoline-6-sulfonic acid]-diammonium salt; which turns green after reaction with HRP), 2,2′-azino-bis(3-ethylbenzothiazoline-6-sulfonic acid (ABTS); 3-amino-9-ethylcarbazole (AEC); 3,3′Diaminobenzidine (DAB); StayYellow (AbCam™ product); and 4-chloro-1-napthol (4-CN, or CN). Exemplary substrates for alkaline phosphatase include PNPP (p-Nitrophenyl Phosphate, Disodium Salt; which turns yellow after reaction with alkaline phosphatase), 5-bromo-4-chloro-3-indolyl phosphate (BCIP) and p-nitroblue tetrazolium chloride (NBT); Stay Green (AbCam™ product); and 4-Chloro-2-methyl benzenediazonium (aka Fast Red). Exemplary substrates for β-Gal include o-nitrophenyl-β-D-galactopyranoside (ONPG) and 5-Bromo-4-Chloro-3-indolyl-B-D-Galactopyranoside (X-Gal). Exemplary substrates for GO include 2,2′,5-5′-tetra-p-nitrophenyl-3,3′-(3,3′-dimethoxy-4,4′-biphenylene)-di tetrazolium chloride (t-NBT). A preferred enzyme has a fast and steady turnover rate.

When desirable, a label and a binding agent may be linked, for example, covalently associated, by a linker, for example, a cleavable linker, e.g., a photocleavable linker, an enzyme cleavable linker. A photocleavable linker is a linker that can be cleaved by exposure to electromagnetic radiation (e.g., visible light, UV light, or infrared light). The wavelength of light necessary to photocleave the linker depends upon the structure of the photocleavable linker used. Exemplary photocleavable linkers include, but are not limited to, chemical molecules containing an o-nitrobenzyl moiety, a p-nitrobenzyl moiety, a m-nitrobenzyl moiety, a nitroindoline moiety, a bromo hydroxycoumarin moiety, a bromo hydroxyquinoline moiety, a hydroxyphenacyl moiety, a dimethoxybenzoin moiety, or any combinations thereof. Exemplary enzyme cleavable linkers include, but are not limited to, DNA, RNA, peptide linkers, β-glucuronide linkers, or any combinations thereof.

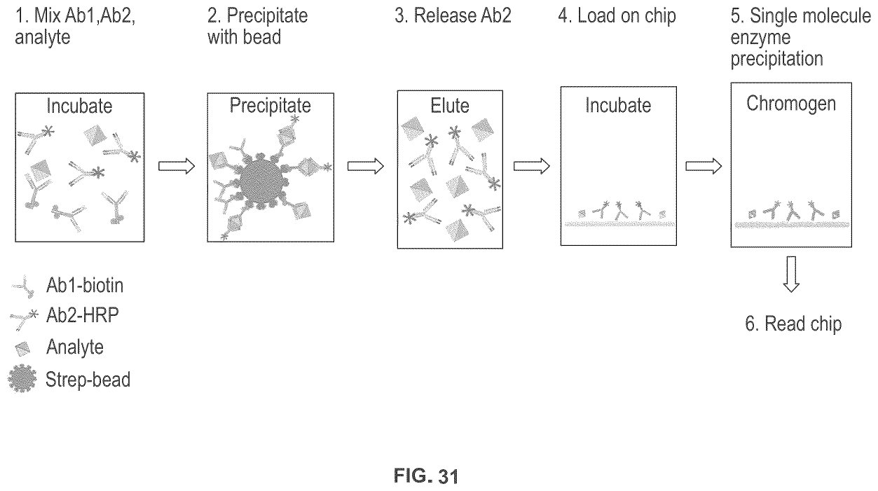

FIG. 31 illustrates an exemplary analyte quantification assay that includes a first antibody which is labeled with biotin (Ab1) and a second antibody that is labeled with HRP (Ab2). Neither antibody is immobilized on a nanostructure at this stage. Each antibody binds to the target analyte, for example, via separate epitopes on the analyte. Incubation of the first antibody, second antibody, and analyte results in the formation of a sandwich complex (see, Step 1). The sandwich complex is then captured by an avidin or streptavidin coated surface (e.g., streptavidin coated beads) that binds to the biotin conjugated to Ab1 (see, Step 2). It is contemplated that this capture strategy captures more analyte than would otherwise be captured by directly capturing the analyte with an antibody pre-immobilized (e.g., coated) on a solid surface. After a washing step, if desired, the Ab2 is eluted from the streptavidin surface (see, Step 3) by changing the solution conditions (e.g., by changing pH, salt concentration or temperature) and then applied to an activated (but not functionalized) nanostructure or series of activated nanostructures (see, Step 4) whereupon the eluted Ab2 molecules are captured by the activated nanostructures. A HRP substrate (e.g., TMB) then is applied to the nanostructure or series of nanostructures, which is then catalytically converted into product (e.g., a precipitate) formed on the nanostructure or series of nanostructures which creates a detectable signal (see, Step 5), which can then be detected by the system (see, Step 6).

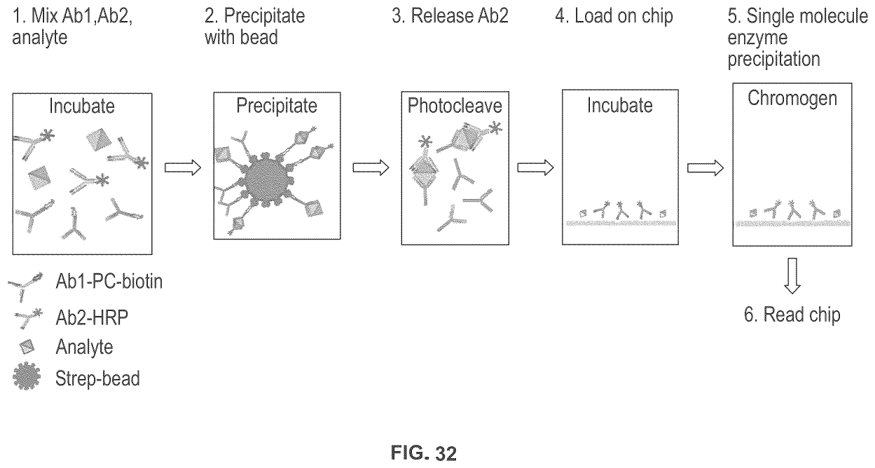

FIG. 32 illustrates another exemplary analyte quantification assay including a first antibody which is labeled with biotin (Ab1) and a second antibody which is labeled with HRP (Ab2). Ab1 is covalently linked to the biotin via a photocleavable linker. Each antibody binds to the target analyte. Incubation of the first antibody, second antibody, and analyte results in the formation of a sandwich complex (see, Step 1). The sandwich complex is then captured by an avidin or streptavidin coated surface (e.g., a streptavidin coated bead) that binds to the biotin on Ab1 (see, Step 2). After enrichment and washing, if desired, the photocleavable linker is then cleaved, removing the sandwich complex from the streptavidin surface (see, Step 3), and the complex is applied to an activated nanostructure or series of activated nanostructures (see, Step 4) whereupon the Ab2 or Ab2 containing complexes are captured by the activated nanostructure(s). A HRP substrate (e.g., TMB) then is applied to the nanostructure or series of nanostructures, which is then catalytically converted into product (e.g., a precipitate) formed on the nanostructure or series of nanostructures which creates a detectable signal (see, Step 5), which can then be detected by the system (see, Step 6).

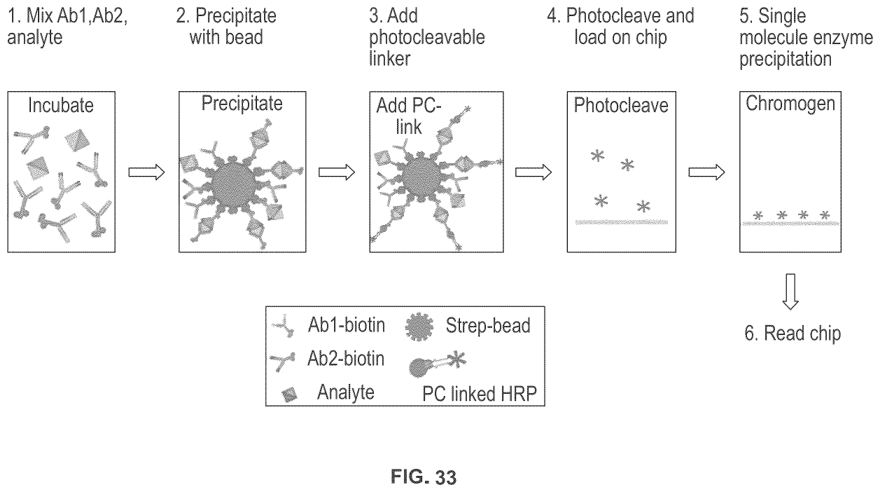

FIG. 33 illustrates another exemplary analyte quantification assay that includes a first antibody that is labeled with biotin (Ab1) and a second antibody which is labeled with biotin (Ab2). Each antibody binds to the target analyte. Incubation of the first antibody, second antibody, and analyte results in the formation of a sandwich complex (see, Step 1). The sandwich complex is then captured by an avidin or streptavidin coated surface (e.g., a streptavidin coated bead) that binds to the biotin on Ab1 or Ab2 (see, Step 2). Then, HRP covalently linked to streptavidin via a photocleavable linker is added (Step 3), which binds to the free biotin on Ab1 or Ab2. After enrichment and washing, if appropriate, the photocleavable linker is cleaved to release the HRP, which is then applied to and captured by an activated nanostructure or series of activated nanostructures (see, Step 4). The addition of a HRP substrate creates a product (e.g., a precipitate) on the surface of a nanostructure or series of nanostructures which creates a detectable signal (see, Step 5), which can then be detected by the system (see, Step 6).

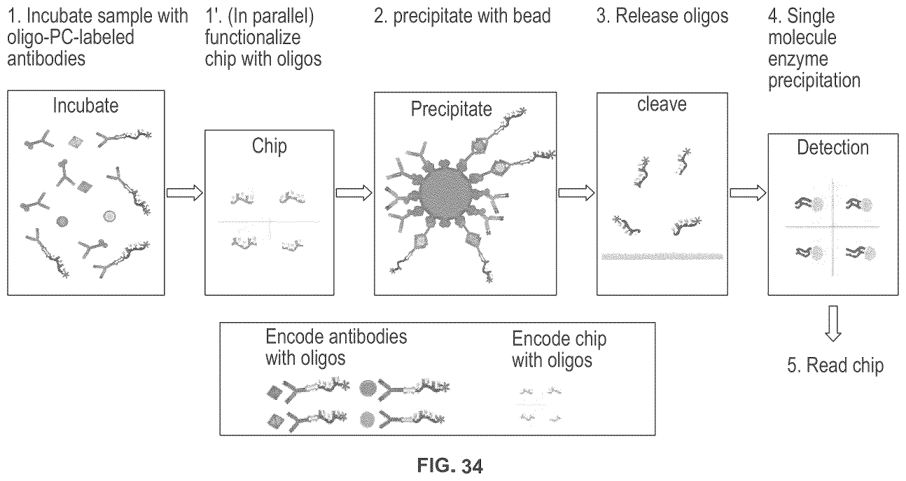

FIG. 34 illustrates another exemplary analyte quantification assay that includes a first antibody that is labeled with (for example, covalently coupled to) biotin and a second antibody that is labeled with (for example, covalently coupled to) an oligonucleotide. The oligonucleotide is linked to the antibody by a cleavable linker located at one end of e.g., a fluorophore or enzyme). The cleavable linker can be an uracil or a plural of uracil inserted at one end of the oligonucleotide. The oligonucleotide can serve as a bar code to the target analyte in Step 1. This can be performed with antibodies that bind to different analytes to facilitate a multiplexing reaction. Each antibody binds to the target analyte if present in the sample. Incubation of the first antibody, second antibody, and analyte results in the formation of a sandwich complex (see, Step 1). In parallel, the nanostructure or series of nanostructures can be functionalized with oligonucleotides complimentary to the oligonucleotides that act as a bar code for each analyte to be detected (see, Step 1′). The sandwich complex is then captured by a streptavidin coated surface (e.g., a streptavidin coated bead) that binds to the biotin on the first antibody (see, Step 2). After enrichment, and washing, as appropriate, the oligonucleotides in each complex can be released by cleavage of the cleavable linkers (see, Step 3), which are applied to and captured by the complementary oligonucleotides attached to the nanostructure or series of nanostructures (see, Step 4), which is then detected by the system (Step 5). The identity and/or concentration of the analyte can be determined from the bar code oligonucleotides captured by the complementary oligonucleotides disposed on the surface of the nanostructure.

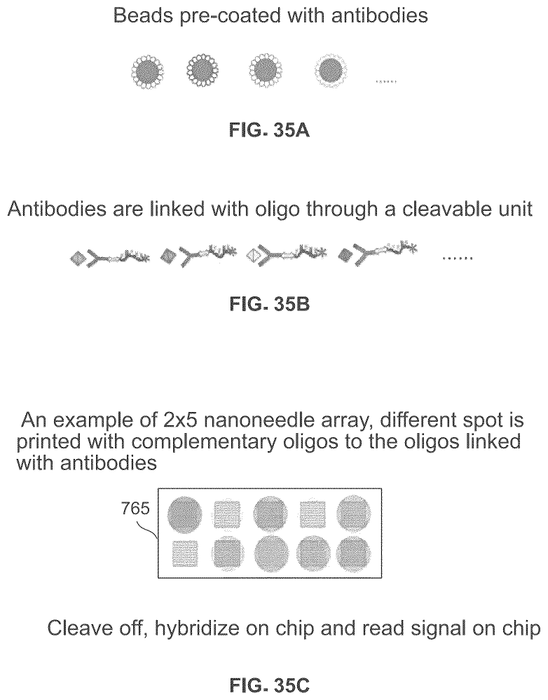

FIG. 35 illustrates reagents for an exemplary multiplex detection assay. For example, a plurality of individual beads are coated with a corresponding plurality of capture antibodies Ab1, Ab2, Ab3 etc. that bind to a corresponding plurality of target analytes (FIG. 35A). A corresponding plurality of detection antibodies labeled with oligonucleotides (bar code for analyte) via a cleavable (for example, a photocleavable) linker (see, FIG. 35B) and then combined with the particles. FIG. 35C represents a sensor 765 with 2×5 nanostructure array, where different regions contain capture oligonucleotides complementary to the corresponding bar code oligonucleotides. The beads are combined and mixed with sample. After the sandwich complexes are permitted to form, the beads are washed and the oligonucleotides are released by cleavage of the cleavable linker. The released bar code oligonucleotides (either with or without a label) are then applied to the sensor with the regions of the capture oligonucleotides (see, FIG. 35D), which are captured and detected as appropriate. The number of antibody coated beads, number of oligonucleotide labeled antibodies and number of oligonucleotide printed regions can be scaled depending upon the desired assay to be performed.

Throughout the description, where compositions (for example, sensors, cartridges or systems) are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition (for example, a sensor, cartridge or system) or a method described herein can be combined in a variety of ways without departing from the spirit and scope of the present invention, whether explicit or implicit herein. For example, where reference is made to a particular feature, that feature can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1—Construction of the Continuous Human Protein-Coding Genome & Unbiased Selection of 100-Plex Protein Panel for Sensor

This example describes the generation of an unbiased 100-protein panel spanning the human protein-coding genome.

For establishment of the human protein-coding genome, based on Piovesan's Gene Table (BMC Res Notes (2019) 12:315, Piovesan et. al., incorporated by reference herein), the Gene ID, Gene symbol, Chromosome accession number, start and end locations of all protein-coding genes were recorded and displayed in the order of their location in the human genome, from chromosome 1 to chromosomes X and Y. All the protein coding genes were then spliced together for continuous numbering of the exome, which resulted in a total length of 1,255,970,826 bp. A sample of the resultant protein-coding genome is shown in Table 4.

TABLE 4

| Gene | Gene | Chr | Chr | Chr | Chr | Gene | Cumu−Gene_ |
|---|---|---|---|---|---|---|---|
| ID | Symbol | Accession | Start | End | Strand | Length bp | Length |
| 729759 | OR4F29 | NC_000001.11 | 50740 | 451678 | − | 939 | 939 |
| 148398 | SAMD11 | NC_000001.11 | 25741 | 944581 | + | 18841 | 19780 |
| 26155 | NOC2L | NC_000001.11 | 44203 | 959299 | − | 15097 | 34498 |
| 339451 | KLHL17 | NC_000001.11 | 960103 | 965719 | + | 5617 | 94953 |
| 84069 | PLEKHN1 | NC_000001.11 | 965820 | 975108 | + | 9289 | 144357 |
| 84808 | PERM1 | NC_000001.11 | 975198 | 982117 | − | 6920 | 200681 |
| 57801 | HES4 | NC_000001.11 | 998962 | 1001052 | − | 2091 | 259096 |
| 9636 | ISG15 | NC_000001.11 | 1013467 | 1014540 | + | 1074 | 318585 |
| 375790 | AGRN | NC_000001.11 | 1020102 | 1056119 | + | 36018 | 414092 |
| 401934 | RNF223 | NC_000001.11 | 1071746 | 1074307 | − | 2562 | 512161 |
| 54991 | Clorf159 | NC_000001.11 | 1081811 | 1116361 | − | 34551 | 644781 |
| 254173 | TTLL10 | NC_000001.11 | 1173709 | 1197936 | + | 24228 | 801629 |
| 8784 | TNFRSF18 | NC_000001.11 | 1203508 | 1207901 | − | 4394 | 962871 |
| 7293 | TNFRSF4 | NC_000001.11 | 1211326 | 1216812 | − | 5487 | 1129600 |
| 51150 | SDF4 | NC_000001.11 | 1216908 | 1232067 | − | 15160 | 1311489 |
| 126792 | B3GALT6 | NC_000001.11 | 1232249 | 1235041 | + | 2793 | 1496171 |
| 388581 | C1QTNF12 | NC_000001.11 | 1242446 | 1247218 | − | 4773 | 1685626 |
| 118424 | UBE2J2 | NC_000001.11 | 1253912 | 1273854 | − | 19943 | 1895024 |
| 6339 | SCNN1D | NC_000001.11 | 1280436 | 1292029 | + | 11594 | 2116016 |
| 116983 | ACAP3 | NC_000001.11 | 1292384 | 1307925 | − | 15542 | 2352550 |
| 126789 | PUSL1 | NC_000001.11 | 1308563 | 1311677 | + | 3115 | 2592199 |
| 54973 | INTS11 | NC_000001.11 | 1311585 | 1324687 | − | 13103 | 2844765 |
| 80772 | CPTP | NC_000001.11 | 1324757 | 1328896 | + | 4140 | 3101564 |
| 83756 | TAS1R3 | NC_000001.11 | 1331324 | 1335320 | + | 3997 | 3362360 |
| 1855 | DVL1 | NC_000001.11 | 1335278 | 1349142 | − | 13865 | 3636935 |
| 54587 | MXRA8 | NC_000001.11 | 1352689 | 1363541 | − | 10853 | 3922406 |
| 54998 | AURKAIP1 | NC_000001.11 | 1373730 | 1375516 | − | 1787 | 4209664 |
| 81669 | CCNL2 | NC_000001.11 | 1385711 | 1399342 | − | 13632 | 4510554 |
| 55052 | MRPL20 | NC_000001.11 | 1401896 | 1407313 | − | 5418 | 4816862 |
| 441869 | ANKRD65 | NC_000001.11 | 1418420 | 1422471 | − | 4052 | 5127222 |
| 643965 | TMEM88B | NC_000001.11 | 1426128 | 1427787 | + | 1660 | 5439242 |
| 64856 | VWA1 | NC_000001.11 | 1435523 | 1442882 | + | 7360 | 5758622 |
| 219293 | ATAD3C | NC_000001.11 | 1449689 | 1470158 | + | 20470 | 6098472 |
| 83858 | ATAD3B | NC_000001.11 | 1471732 | 1509466 | + | 37735 | 6476057 |
| 55210 | ATAD3A | NC_000001.11 | 1512143 | 1534687 | + | 22545 | 6876187 |
| 339453 | TMEM240 | NC_000001.11 | 1534778 | 1540360 | − | 5583 | 7281900 |
| 29101 | SSU72 | NC_000001.11 | 1541673 | 1574882 | − | 33210 | 7720823 |
| 643988 | FNDC10 | NC_000001.11 | 1598008 | 1600096 | − | 2089 | 8161835 |
| 142678 | MIB2 | NC_000001.11 | 1613730 | 1630610 | + | 16881 | 8619728 |
| 8510 | MMP23B | NC_000001.11 | 1631681 | 1635638 | + | 3958 | 9081579 |
| 984 | CDK11B | NC_000001.11 | 1635226 | 1659619 | − | 24394 | 9566998 |
| 728661 | SLC35E2B | NC_000001.11 | 1659798 | 1692804 | − | 33007 | 10085837 |
| 728642 | CDK11A | NC_000001.11 | 1702383 | 1724565 | − | 22183 | 10626859 |
| 9906 | SLC35E2A | NC_000001.11 | 1724838 | 1745999 | − | 21162 | 11189043 |
| 65220 | NADK | NC_000001.11 | 1751232 | 1780509 | − | 29278 | 11780505 |
| 2782 | GNB1 | NC_000001.11 | 1785285 | 1891117 | − | 105833 | 12477800 |
| 163688 | CALML6 | NC_000001.11 | 1913751 | 1917294 | + | 3544 | 13178639 |
| 339456 | TMEM52 | NC_000001.11 | 1917590 | 1919301 | − | 1712 | 13881190 |
| 85452 | CFAP74 | NC_000001.11 | 1921951 | 2003837 | − | 81887 | 14665628 |
| 2563 | GABRD | NC_000001.11 | 2019329 | 2030753 | + | 11425 | 15461491 |
| 5590 | PRKCZ | NC_000001.11 | 2050470 | 2185399 | + | 134930 | 16392284 |
| 199990 | FAAP20 | NC_000001.11 | 2184457 | 2212720 | − | 28264 | 17349455 |
| 6497 | SKI | NC_000001.11 | 2228695 | 2310213 | + | 81519 | 18389088 |
| 79906 | MORN1 | NC_000001.11 | 2319202 | 2391751 | − | 72550 | 19501271 |
| 11079 | RER1 | NC_000001.11 | 2391292 | 2405444 | + | 14153 | 20626687 |
| 5192 | PEX10 | NC_000001.11 | 2404802 | 2412574 | − | 7773 | 21759050 |
| 9651 | PLCH2 | NC_000001.11 | 2412481 | 2505530 | + | 93050 | 22984918 |
| 55229 | PANK4 | NC_000001.11 | 2508531 | 2526628 | − | 18098 | 24228978 |
| 388585 | HES5 | NC_000001.11 | 2528745 | 2530554 | − | 1810 | 25474848 |
| 8764 | TNFRSF14 | NC_000001.11 | 2556365 | 2565622 | + | 9258 | 26729976 |
| 127281 | PRXL2B | NC_000001.11 | 2586460 | 2591469 | + | 5010 | 27990114 |
| 79258 | MMEL1 | NC_000001.11 | 2590639 | 2633042 | − | 42404 | 29290994 |
| 100287898 | TTC34 | NC_000001.11 | 2636988 | 2789737 | − | 152750 | 30745455 |
| 140625 | ACTRT2 | NC_000001.11 | 3021482 | 3022903 | + | 1422 | 32201338 |
| 63976 | PRDM16 | NC_000001.11 | 3068227 | 3438621 | + | 370395 | 34027616 |
| 27237 | ARHGEF16 | NC_000001.11 | 3454583 | 3481115 | + | 26533 | 35880427 |
| 1953 | MEGF6 | NC_000001.11 | 3487942 | 3624770 | − | 136829 | 37870067 |
| 127262 | TPRG1L | NC_000001.11 | 3624992 | 3630131 | + | 5140 | 39864847 |
| 49856 | WRAP73 | NC_000001.11 | 3630767 | 3650107 | − | 19341 | 41878968 |
| 7161 | TP73 | NC_000001.11 | 3652565 | 3736201 | + | 83637 | 43976726 |
| 148870 | CCDC27 | NC_000001.11 | 3752398 | 3771645 | + | 19248 | 46093732 |
| 388588 | SMIM1 | NC_000001.11 | 3772761 | 3775982 | + | 3222 | 48213960 |
| 57470 | LRRC47 | NC_000001.11 | 3780220 | 3796504 | − | 16285 | 50350473 |
| 9731 | CEP104 | NC_000001.11 | 3812081 | 3857233 | − | 45153 | 52532139 |
| 1677 | DFFB | NC_000001.11 | 3857267 | 3885429 | + | 28163 | 54741968 |
| 339448 | Clorf174 | NC_000001.11 | 3889133 | 3900293 | − | 11161 | 56962958 |
| 55966 | AJAP1 | NC_000001.11 | 4654775 | 4792518 | + | 137744 | 59321692 |
| 261734 | NPHP4 | NC_000001.11 | 5862808 | 5992473 | − | 129666 | 61810092 |
| 8514 | KCNAB2 | NC_000001.11 | 5992298 | 6101193 | + | 108896 | 64407036 |
| 26038 | CHD5 | NC_000001.11 | 6101787 | 6180134 | − | 78348 | 67082504 |
| 6146 | RPL22 | NC_000001.11 | 6185020 | 6199619 | − | 14600 | 69772572 |
| 388591 | RNF207 | NC_000001.11 | 6206004 | 6221299 | + | 15296 | 72477936 |
| 23463 | ICMT | NC_000001.11 | 6221193 | 6235984 | − | 14792 | 75197878 |
| 390992 | HES3 | NC_000001.11 | 6244192 | 6245578 | + | 1387 | 77919314 |
| 387509 | GPR153 | NC_000001.11 | 6247346 | 6260975 | − | 13630 | 80654380 |
| 442868 | BPY2C | NC_000024.10 | 25030901 | 25052104 | − | 21204 | 1,255,967,409 |
| 9085 | CDY1 | NC_000024.10 | 25622095 | 25625511 | + | 3417 | 1,255,970,826 |

To construct a 100-plex protein panel in a bias-free manner, 100 position markers were placed along the spliced genes, starting at 12,559,708 bp, with each marker located at 12,559,708*i, where i is the sequence of the marker. The spacing between the markers was 12,559,708 bp. For the i^{th} marker, using Single Nucleotide Polymorphism Database (dbSNP), a SNIP that is nearest to the position marker I was located. Then, the gene that contains the identified SNIP was located and included in the panel as the i^{th} protein. A panel of 100 proteins is listed in Table 5.

TABLE 5

| | | Chromosome | | | Gene Length |
|---|---|---|---|---|---|
| Gene ID | Gene Symbol | Accession | Chr Start | Chr End | bp |
| 249 | ALPL | NC_000001.11 | 21508982 | 21578412 | 69431 |
| 11004 | KIF2C | NC_000001.11 | 44739704 | 44767767 | 28064 |
| 9829 | DNAJC6 | NC_000001.11 | 65264694 | 65415869 | 151176 |
| 343099 | CCDC18 | NC_000001.11 | 93179802 | 93279037 | 99236 |
| 81611 | ANP32E | NC_000001.11 | 150218417 | 150236156 | 17740 |
| 27101 | CACYBP | NC_000001.11 | 174999435 | 175012027 | 12593 |
| 6900 | CNTN2 | NC_000001.11 | 205042937 | 205078272 | 35336 |
| 7257 | TSNAX | NC_000001.11 | 231528653 | 231566524 | 37872 |
| 3241 | HPCAL1 | NC_000002.12 | 10302124 | 10427617 | 125494 |
| 27436 | EML4 | NC_000002.12 | 42169338 | 42332548 | 163211 |
| 27332 | ZNF638 | NC_000002.12 | 71276593 | 71435061 | 158469 |
| 3987 | LIMS1 | NC_000002.12 | 108534355 | 108687246 | 152892 |
| 80731 | THSD7B | NC_000002.12 | 136765562 | 137677712 | 912151 |
| 79809 | TTC21B | NC_000002.12 | 165873362 | 165953838 | 80477 |
| 23671 | TMEFF2 | NC_000002.12 | 191948300 | 192194933 | 246634 |
| 3488 | IGFBP5 | NC_000002.12 | 216672105 | 216695549 | 23445 |
| 152330 | CNTN4 | NC_000003.12 | 2098803 | 3059080 | 960278 |
| 152110 | NEK10 | NC_000003.12 | 27107336 | 27369460 | 262125 |
| 1795 | DOCK3 | NC_000003.12 | 50674969 | 51384198 | 709230 |
| 9039 | UBA3 | NC_000003.12 | 69054730 | 69080373 | 25644 |
| 10225 | CD96 | NC_000003.12 | 111542079 | 111665996 | 123918 |
| 131034 | CPNE4 | NC_000003.12 | 131533560 | 132285696 | 752137 |
| 100505385 | IQCJ-SCHIP1 | NC_000003.12 | 159069252 | 159897366 | 828115 |
| 8626 | TP63 | NC_000003.12 | 189596746 | 189897279 | 300534 |
| 80333 | KCNIP4 | NC_000004.12 | 20728520 | 21948751 | 1220232 |
| 23284 | ADGRL3 | NC_000004.12 | 61201256 | 62078335 | 877080 |
| 401145 | CCSER1 | NC_000004.12 | 90127394 | 91602219 | 1474826 |
| 54532 | USP53 | NC_000004.12 | 119212583 | 119295518 | 82936 |
| 56884 | FSTL5 | NC_000004.12 | 161383892 | 162164034 | 780143 |
| 170690 | ADAMTS16 | NC_000005.10 | 5140330 | 5320304 | 179975 |
| 23530 | NNT | NC_000005.10 | 43601092 | 43705566 | 104475 |
| 10184 | LHFPL2 | NC_000005.10 | 78485215 | 78770256 | 285042 |
| 1176 | AP3S1 | NC_000005.10 | 115841606 | 115914081 | 72476 |
| 9752 | PCDHA9 | NC_000005.10 | 140847772 | 141012344 | 164573 |
| 2561 | GABRB2 | NC_000005.10 | 161288429 | 161548124 | 259696 |
| 84830 | ADTRP | NC_000006.12 | 11713523 | 11779628 | 66106 |
| 25862 | USP49 | NC_000006.12 | 41789896 | 41895361 | 105466 |
| 57579 | FAM135A | NC_000006.12 | 70413404 | 70561174 | 147771 |
| 221264 | AK9 | NC_000006.12 | 109492855 | 109691212 | 198358 |
| 57224 | NHSL1 | NC_000006.12 | 138422043 | 138692616 | 270574 |
| 5071 | PRKN | NC_000006.12 | 161347417 | 162727802 | 1380386 |
| 9734 | HDAC9 | NC_000007.14 | 18086942 | 19002416 | 915475 |
| 644 | BLVRA | NC_000007.14 | 43758122 | 43807342 | 49221 |
| 781 | CACNA2D1 | NC_000007.14 | 81946444 | 82443806 | 497363 |
| 100130771 | EFCAB10 | NC_000007.14 | 105565108 | 105581864 | 16757 |
| 60412 | EXOC4 | NC_000007.14 | 133253067 | 134067137 | 814071 |
| 155435 | RBM33 | NC_000007.14 | 155644494 | 155781485 | 136992 |
| 55806 | HR | NC_000008.11 | 22114419 | 22133384 | 18966 |
| 8601 | RGS20 | NC_000008.11 | 53851808 | 53959304 | 107497 |
| 168975 | CNBD1 | NC_000008.11 | 86866442 | 87428652 | 562211 |
| 401474 | SAMD12 | NC_000008.11 | 118131828 | 118621995 | 490168 |
| 57589 | RIC1 | NC_000009.12 | 5629030 | 5778633 | 149604 |
| 23349 | PHF24 | NC_000009.12 | 34810040 | 34982544 | 172505 |
| 65268 | WNK2 | NC_000009.12 | 93184156 | 93327581 | 143426 |
| 158135 | TTLL11 | NC_000009.12 | 121815674 | 122093606 | 277933 |
| 5588 | PRKCQ | NC_000010.11 | 6393038 | 6580646 | 187609 |
| 6840 | SVIL | NC_000010.11 | 29457338 | 29737001 | 279664 |
| 22891 | ZNF365 | NC_000010.11 | 62374157 | 62672011 | 297855 |
| 2894 | GRID1 | NC_000010.11 | 85599555 | 86366493 | 766939 |
| 114815 | SORCS1 | NC_000010.11 | 106573663 | 107181138 | 607476 |
| 3784 | KCNQ1 | NC_000011.10 | 2444991 | 2849110 | 404120 |
| 63982 | ANO3 | NC_000011.10 | 26189123 | 26667907 | 478785 |
| 28992 | MACROD1 | NC_000011.10 | 63998554 | 64166652 | 168099 |
| 51501 | HIKESHI | NC_000011.10 | 86302211 | 86345943 | 43733 |
| 23705 | CADM1 | NC_000011.10 | 115173625 | 115504523 | 330899 |
| 56341 | PRMT8 | NC_000012.12 | 3381349 | 3593973 | 212625 |
| 55297 | CCDC91 | NC_000012.12 | 28190427 | 28550166 | 359740 |
| 121227 | LRIG3 | NC_000012.12 | 58872155 | 58920538 | 48384 |
| 8411 | EEA1 | NC_000012.12 | 92772509 | 92975228 | 202720 |
| 23389 | MED13L | NC_000012.12 | 115958576 | 116277219 | 318644 |
| 51761 | ATP8A2 | NC_000013.11 | 25372011 | 26025851 | 653841 |
| 55901 | THSD1 | NC_000013.11 | 52377167 | 52406494 | 29328 |
| 2259 | FGF14 | NC_000013.11 | 101720855 | 102402428 | 681574 |
| 57697 | FANCM | NC_000014.9 | 45135939 | 45200890 | 64952 |
| 64093 | SMOC1 | NC_000014.9 | 69879388 | 70032366 | 152979 |
| 145270 | PRIMA1 | NC_000014.9 | 93718298 | 93789029 | 70732 |
| 56924 | PAK6 | NC_000015.10 | 40239091 | 40277487 | 38397 |
| 6095 | RORA | NC_000015.10 | 60488284 | 61229303 | 741020 |
| 79631 | EFL1 | NC_000015.10 | 82130220 | 82262763 | 132544 |
| 54715 | RBFOX1 | NC_000016.10 | 5239752 | 7713343 | 2473592 |
| 83985 | SPNS1 | NC_000016.10 | 28973999 | 28984769 | 10771 |
| 388289 | C16orf47 | NC_000016.10 | 73125865 | 73891931 | 766067 |
| 9135 | RABEP1 | NC_000017.11 | 5282263 | 5386340 | 104078 |
| 40 | ASIC2 | NC_000017.11 | 33013087 | 34156806 | 1143720 |
| 6827 | SUPT4H1 | NC_000017.11 | 58345175 | 58352238 | 7064 |
| 125058 | TBC1D16 | NC_000017.11 | 79932343 | 80035875 | 103533 |
| 1000 | CDH2 | NC_000018.10 | 27932878 | 28177446 | 244569 |
| 10892 | MALT1 | NC_000018.10 | 58671386 | 58753806 | 82421 |
| 1786 | DNMT1 | NC_000019.10 | 10133344 | 10195135 | 61792 |
| 6261 | RYR1 | NC_000019.10 | 38433700 | 38587564 | 153865 |
| 55968 | NSFLIC | NC_000020.11 | 1442162 | 1467793 | 25632 |
| 9139 | CBFA2T2 | NC_000020.11 | 33490070 | 33650031 | 159962 |
| 60437 | CDH26 | NC_000020.11 | 59957764 | 60037971 | 80208 |
| 54097 | FAM3B | NC_000021.9 | 41304229 | 41357727 | 53499 |
| 8224 | SYN3 | NC_000022.11 | 32507820 | 33058391 | 550572 |
| 3730 | ANOS1 | NC_000023.11 | 8528874 | 8732187 | 203314 |
| 347404 | LANCL3 | NC_000023.11 | 37571569 | 37684463 | 112895 |
| 546 | ATRX | NC_000023.11 | 77504878 | 77786269 | 281392 |
| 23157 | SEPT6 | NC_000023.11 | 119615724 | 119693370 | 77647 |
| 9085 | CDY1 | NC_000024.10 | 25622095 | 25625511 | 3417 |

### Example 2—Protein Analysis of a Patient Sample Using a 100-Plex Protein Panel for Sensor

This example describes the testing of a patient sample of an unbiased 100-protein panel of the human protein-coding genome.

An exemplary 100-plex protein panel (e.g., Table 5) is designed and antibodies specific to each protein are selected. A sensor plate layout is shown in FIG. 1E. The wells are placed in a SBS-96 format, and each well contains a 10 by 10 grid. Each grid has a nanostructure array. All wells are activated by glutaraldehyde and (3-aminopropyl)-trimethoxysilane (APTMS). Next, antibodies specific to each of the proteins in the 100-plex panel are functionalized on the respective sensor array in each grid using printing technologies. Each 96 plate contains 96 wells, which can run 48 samples in duplicate. Plasma or serum samples from a test group, for example, a group of subjects to be interrogated for protein associations to a phenotype (e.g., a disease group) and control group are added to the wells. Digital and analog signals from each of the sensor arrays are analyzed to cover a large dynamic range of protein concentrations. The protein concentrations from the control and test groups are compared. A set of biomarkers is thus identified to best differentiate the test group from the control group.

### Example 3—Construction of the Continuous Human Exome Excluding Introns & Unbiased Selection of 100-Plex Protein Panel for Sensor

This example describes the generation of an unbiased 100-protein panel spanning the human exome (which excludes intron sequences).

A protein panel was constructed from an exome (i.e., excluding the introns from the protein coding genes). One isoform of a protein was chosen from Piovesan's Gene Table (described above), and the start and end locations of the 3′ UTR3, CDS and 5′ UTR were noted to mark the exons. All exons were then spliced together, which resulted in a total exome length of 62,184,186 bp. A sample of the resultant exome is shown in Table 6

TABLE 6

| | | | | | | | | | | | UTR3′_ | UTR3′_ |
|---|---|---|---|---|---|---|---|---|---|---|---|---|
| Gene | Gene | Chr | Chr | Chr | CDS_ | CDS_ | UTR5′_ | UTR5′_ | UTR3′_ | UTR3′_ | Length_ | Length_ |
| ID | Symbol | Accession | Start | End | Start | End | Start | End | Start | End | bp | bp |
| 729759 | OR4F29 | NC_ | 50740 | 451678 | 450740 | 451678 | 451679 | 451678 | 450740 | 450739 | 0 | 1 |
| | | 000001.11 | | | | | | | | | | |
| 48398 | SAMD11 | NC_ | 25741 | 944581 | 925942 | 944153 | 925741 | 925941 | 944154 | 944581 | 428 | 429 |
| | | 000001.11 | | | | | | | | | | |
| 26155 | NOC2L | NC_ | 44203 | 959299 | 944694 | 959240 | 959241 | 959299 | 944203 | 944693 | 491 | 492 |
| | | 000001.11 | | | | | | | | | | |
| 339451 | KLHL17 | NC_ | 960103 | 965719 | 960694 | 965191 | 960587 | 960693 | 965192 | 965719 | 528 | 529 |
| | | 000001.11 | | | | | | | | | | |
| 84069 | PLEKHN1 | NC_ | 965820 | 975108 | 966532 | 974575 | 966497 | 966531 | 974576 | 975108 | 533 | 534 |
| | | 000001.11 | | | | | | | | | | |
| 84808 | PERM1 | NC_ | 975198 | 982117 | 976172 | 981029 | 981030 | 982117 | 975199 | 976171 | 973 | 974 |
| | | 000001.11 | | | | | | | | | | |
| 57801 | HES4 | NC_ | 998962 | 1001052 | | | | | | | | |
| | | 000001.11 | | | | | | | | | | |
| 9636 | ISG15 | NC_ | 1013467 | 1014540 | 1013574 | 1014478 | 1013467 | 1013573 | 1014479 | 1014540 | 62 | 63 |
| | | 000001.11 | | | | | | | | | | |
| 375790 | AGRN | NC_ | 1020102 | 1056119 | 1020173 | 1054981 | 1020123 | 1020172 | 1054982 | 1056119 | 1138 | 1139 |
| | | 000001.11 | | | | | | | | | | |
| 401934 | RNF223 | NC_ | 1071746 | 1074307 | 1071817 | 1072566 | 1072567 | 1074307 | 1071746 | 1071816 | 71 | 72 |
| | | 000001.11 | | | | | | | | | | |
| 54991 | Clorf159 | NC_ | 1081811 | 1116361 | 1082893 | 1091543 | 1091544 | 1116356 | 1081818 | 1082892 | 1075 | 1076 |
| | | 000001.11 | | | | | | | | | | |
| 254173 | TTLL 10 | NC_ | 1173709 | 1197936 | 1179216 | 1197847 | 1173906 | 1179215 | 1197848 | 1197933 | 86 | 87 |
| | | 000001.11 | | | | | | | | | | |
| 8784 | TNFRSF18 | NC_ | 1203508 | 1207901 | 1203591 | 1206571 | 1206572 | 1206709 | 1203508 | 1203590 | 83 | 84 |
| | | 000001.11 | | | | | | | | | | |
| 7293 | TNFRSF4 | NC_ | 1211326 | 1216812 | 1211555 | 1214127 | 1214128 | 1214168 | 1211326 | 1211554 | 229 | 230 |
| | | 000001.11 | | | | | | | | | | |
| 51150 | SDF4 | NC_ | 1216908 | 1232067 | 1217512 | 1228793 | 1228794 | 1232067 | 1216908 | 1217511 | 604 | 605 |
| | | 000001.11 | | | | | | | | | | |
| 126792 | B3GALT6 | NC_ | 1232249 | 1235041 | 1232279 | 1233268 | 1232249 | 1232278 | 1233269 | 1235041 | 1773 | 1774 |
| | | 000001.11 | | | | | | | | | | |
| 388581 | C1QTNF12 | NC__ | 1242446 | 1247218 | 1242548 | 1246690 | 1246691 | 1246722 | 1242446 | 1242547 | 102 | 103 |
| | | 000001.11 | | | | | | | | | | |
| 118424 | UBE2J2 | NC_ | 1253912 | 1273854 | 1255203 | 1263361 | 1263362 | 1273854 | 1253912 | 1255202 | 1291 | 1292 |
| | | 000001.11 | | | | | | | | | | |
| 6339 | SCNNID | NC_ | 1280436 | 1292029 | 1280662 | 1291610 | 1280436 | 1280661 | 1291611 | 1292029 | 419 | 420 |
| | | 000001.11 | | | | | | | | | | |
| 116983 | ACAP3 | NC_ | 1292384 | 1307925 | 1293564 | 1307815 | 1307816 | 1307889 | 1292384 | 1293563 | 1180 | 1181 |
| | | 000001.11 | | | | | | | | | | |
| 126789 | PUSL1 | NC_ | 1308563 | 1311677 | 1308644 | 1311379 | 1308580 | 1308643 | 1311380 | 1311677 | 298 | 299 |
| | | 000001.11 | | | | | | | | | | |
| 54973 | INTS11 | NC_ | 1311585 | 1324687 | 1311859 | 1324608 | 1324609 | 1324687 | 1311585 | 1311858 | 274 | 275 |
| | | 000001.11 | | | | | | | | | | |
| 80772 | CPTP | NC_ | 1324757 | 1328896 | 1326911 | 1327763 | 1324763 | 1326910 | 1327764 | 1328896 | 1133 | 1134 |
| | | 000001.11 | | | | | | | | | | |
| 83756 | TASIR3 | NC__ | 1331324 | 1335320 | 1331346 | 1334464 | 1331346 | 1331345 | 1334465 | 1335320 | 856 | 857 |
| | | 000001.11 | | | | | | | | | | |
| 1855 | DVL1 | NC_ | 1335278 | 1349142 | 1336142 | 1349065 | 1349066 | 1349112 | 1335278 | 1336141 | 864 | 865 |
| | | 000001.11 | | | | | | | | | | |
| 54587 | MXRA8 | NC_ | 1352689 | 1363541 | 1353604 | 1358504 | 1358505 | 1363541 | 1352689 | 1353603 | 915 | 916 |
| | | 000001.11 | | | | | | | | | | |
| 54998 | AURKAIP1 | NC_ | 1373730 | 1375516 | 1373801 | 1374756 | 1374757 | 1375438 | 1373730 | 1373800 | 71 | 72 |
| | | 000001.11 | | | | | | | | | | |
| 81669 | CCNL2 | NC_ | 1385711 | 1399342 | 1387231 | 1399306 | 1399307 | 1399342 | 1385711 | 1387230 | 1520 | 1521 |
| | | 000001.11 | | | | | | | | | | |
| 55052 | MRPL20 | NC_ | 1401896 | 1407313 | 1402083 | 1407217 | 1407218 | 1407313 | 1401896 | 1402082 | 187 | 188 |
| | | 000001.11 | | | | | | | | | | |
| 441869 | ANKRD65 | NC_ | 1418420 | 1422471 | 1419542 | 1421005 | 1421006 | 1421444 | 1418420 | 1419541 | 1122 | 1123 |
| | | 000001.11 | | | | | | | | | | |
| 643965 | TMEM88B | NC_ | 1426128 | 1427787 | 1426128 | 1427787 | 1426128 | 1426127 | 1427788 | 1427787 | 0 | 1 |
| | | 000001.11 | | | | | | | | | | |
| 64856 | VWA1 | NC_ | 1435523 | 1442882 | 1435749 | 1439787 | 1435523 | 1435748 | 1439788 | 1442882 | 3095 | 3096 |
| | | 000001.11 | | | | | | | | | | |
| 219293 | ATAD3C | NC_ | 1449689 | 1470158 | 1450684 | 1468530 | 1449689 | 1450683 | 1468531 | 1470158 | 1628 | 1629 |
| | | 000001.11 | | | | | | | | | | |
| 83858 | ATAD3B | NC_ | 1471732 | 1509466 | 1471885 | 1495817 | 1471755 | 1471884 | 1495818 | 1496204 | 387 | 388 |
| | | 000001.11 | | | | | | | | | | |
| 55210 | ATAD3A | NC_ | 1512143 | 1534687 | 1512269 | 1534072 | 1512143 | 1512268 | 1534073 | 1534687 | 615 | 616 |
| | | 000001.11 | | | | | | | | | | |
| 339453 | TMEM240 | NC_ | 1534778 | 1540360 | 1535359 | 1540346 | 1540347 | 1540360 | 1534778 | 1535358 | 581 | 582 |
| | | 000001.11 | | | | | | | | | | |
| 29101 | SSU72 | NC_ | 1541673 | 1574882 | 1542066 | 1574557 | 1574558 | 1574882 | 1541673 | 1542065 | 393 | 394 |
| | | 000001.11 | | | | | | | | | | |
| 643988 | FNDC10 | NC_ | 1598008 | 1600096 | 1599335 | 1600015 | 1600016 | 1600096 | 1598008 | 1599334 | 1327 | 1328 |
| | | 000001.11 | | | | | | | | | | |
| 142678 | MIB2 | NC_ | 1613730 | 1630610 | 1615460 | 1630530 | 1615415 | 1615459 | 1630531 | 1630610 | 80 | 81 |
| | | 000001.11 | | | | | | | | | | |
| 8510 | MMP23B | NC_ | 1631681 | 1635638 | 1632219 | 1634625 | 1632181 | 1632218 | 1634626 | 1634654 | 29 | 30 |
| | | 000001.11 | | | | | | | | | | |
| 984 | CDK11B | NC_ | 1635226 | 1659619 | 1635764 | 1657485 | 1657486 | 1659097 | 1635226 | 1635763 | 538 | 539 |
| | | 000001.11 | | | | | | | | | | |
| 728661 | SLC35E2B | NC_ | 1659798 | 1692804 | 1665782 | 1676699 | 1676700 | 1692804 | 1661478 | 1665781 | 4304 | 4305 |
| | | 000001.11 | | | | | | | | | | |
| 728642 | CDK11A | NC_ | 1702383 | 1724565 | 1702907 | 1722818 | 1722819 | 1724565 | 1702383 | 1702906 | 524 | 525 |
| | | 000001.11 | | | | | | | | | | |
| 9906 | SLC35E2A | NC_ | 1724838 | 1745999 | 1725414 | 1739557 | 1739558 | 1745999 | 1724838 | 1725413 | 576 | 577 |
| | | 000001.11 | | | | | | | | | | |
| 65220 | NADK | NC_ | 1751232 | 1780509 | 1752904 | 1765406 | 1765407 | 1780147 | 1751232 | 1752903 | 1672 | 1673 |
| | | 000001.11 | | | | | | | | | | |
| 2782 | GNB1 | NC_ | 1785285 | 1891117 | 1787331 | 1825453 | 1825454 | 1891117 | 1785285 | 1787330 | 1778 | 1779 |
| | | 000001.11 | | | | | | | | | | |
| 442868 | BPY2C | NC_ | 25030901 | 25052104 | 25038098 | 25044023 | 25044024 | 25052104 | 25030901 | 25038097 | 553 | 554 |
| | | 000024.10 | | | | | | | | | | |
| 9085 | CDY1 | NC_ | 25622095 | 25625511 | 25622443 | 25624527 | 25622117 | 25622442 | 25624528 | 25624902 | 375 | 376 |
| | | 000024.10 | | | | | | | | | | |

A 100-plex protein panel was generated in a bias-free manner from the above-described exome, by placing 100 position markers along the spliced genes, starting at 621,842 bp, with each marker located at 621,842*I, where I is the sequence of the marker. The spacing between the markers was 621,842 bp. For the i^{th} marker, using the Single Nucleotide Polymorphism Database (dbSNP), a SNP that was nearest to the position marker i was located. Then, the gene containing the identified SNP was located and included in the panel as the i^{th} protein. The resultant protein list generated from the above protocol is shown in Table 7.

TABLE 7

| | | Chromosome | | | Gene Length |
|---|---|---|---|---|---|
| Gene ID | Gene Symbol | Accession | Chr Start | Chr End | bp |
| 55672 | NBPF1 | NC_000001.11 | 16562427 | 16613605 | 51179 |
| 10657 | KHDRBS1 | NC_000001.11 | 32013694 | 32060859 | 47166 |
| 387338 | NSUN4 | NC_000001.11 | 46340177 | 46365152 | 24976 |
| 5567 | PRKACB | NC_000001.11 | 84077975 | 84238498 | 160524 |
| 9860 | LRIG2 | NC_000001.11 | 113073170 | 113132260 | 59091 |
| 2312 | FLG | NC_000001.11 | 152302175 | 152325203 | 23029 |
| 4921 | DDR2 | NC_000001.11 | 162631265 | 162786573 | 155309 |
| 343450 | KCNT2 | NC_000001.11 | 196225779 | 196608576 | 382798 |
| 149643 | SPATA45 | NC_000001.11 | 212830141 | 212847649 | 17509 |
| 64388 | GREM2 | NC_000001.11 | 240489573 | 240612372 | 122800 |
| 84226 | C2orf16 | NC_000002.12 | 27576522 | 27582722 | 6201 |
| 7840 | ALMS1 | NC_000002.12 | 73385758 | 73609919 | 224162 |
| 51263 | MRPL30 | NC_000002.12 | 99181079 | 99199557 | 18479 |
| 84083 | ZRANB3 | NC_000002.12 | 135164218 | 135531236 | 367019 |
| 3232 | HOXD3 | NC_000002.12 | 176157163 | 176173102 | 15940 |
| 940 | CD28 | NC_000002.12 | 203706475 | 203739756 | 33282 |
| 5757 | PTMA | NC_000002.12 | 231708525 | 231713541 | 5017 |
| 9779 | TBC1D5 | NC_000003.12 | 17157162 | 17742739 | 585578 |
| 10201 | NME6 | NC_000003.12 | 48288402 | 48302904 | 14503 |
| 317649 | EIF4E3 | NC_000003.12 | 71679289 | 71754773 | 75485 |
| 165631 | PARP15 | NC_000003.12 | 122575926 | 122639047 | 63122 |
| 116931 | MED12L | NC_000003.12 | 151085665 | 151436677 | 351013 |
| 4026 | LPP | NC_000003.12 | 188152152 | 188890671 | 738520 |
| 80306 | MED28 | NC_000004.12 | 17614628 | 17625628 | 11001 |
| 2926 | GRSF1 | NC_000004.12 | 70815782 | 70843274 | 27493 |
| 27123 | DKK2 | NC_000004.12 | 106921802 | 107036296 | 114495 |
| 1519 | CTSO | NC_000004.12 | 155924118 | 155953896 | 29779 |
| 1004 | CDH6 | NC_000005.10 | 31193655 | 31329146 | 135492 |
| 3842 | TNPO1 | NC_000005.10 | 72816591 | 72914388 | 97798 |
| 1657 | DMXL1 | NC_000005.10 | 119071002 | 119249432 | 178431 |
| 153768 | PRELID2 | NC_000005.10 | 145587325 | 145835369 | 248045 |
| 90249 | UNC5A | NC_000005.10 | 176810559 | 176880898 | 70340 |
| 10279 | PRSS16 | NC_000006.12 | 27247701 | 27256620 | 8920 |
| 60685 | ZFAND3 | NC_000006.12 | 37819531 | 38154624 | 335094 |
| 25821 | MTO1 | NC_000006.12 | 73461731 | 73501456 | 39726 |
| 29940 | DSE | NC_000006.12 | 116254152 | 116441261 | 187110 |
| 54516 | MTRF1L | NC_000006.12 | 152987366 | 153003439 | 16074 |
| 25928 | SOSTDC1 | NC_000007.14 | 16461481 | 16465849 | 4369 |
| 55915 | LANCL2 | NC_000007.14 | 55365448 | 55433742 | 68295 |
| 222865 | TMEM130 | NC_000007.14 | 98846488 | 98870050 | 23563 |
| 168850 | ZNF800 | NC_000007.14 | 127342871 | 127392798 | 49928 |
| 116988 | AGAP3 | NC_000007.14 | 151085831 | 151144436 | 58606 |
| 23087 | TRIM35 | NC_000008.11 | 27284886 | 27311319 | 26434 |
| 63978 | PRDM14 | NC_000008.11 | 70051613 | 70071327 | 19715 |
| 5168 | ENPP2 | NC_000008.11 | 119557077 | 119673576 | 116500 |
| 158358 | KIAA2026 | NC_000009.12 | 5860254 | 6008489 | 148236 |
| 320 | APBA1 | NC_000009.12 | 69427532 | 69673012 | 245481 |
| 83856 | FSD1L | NC_000009.12 | 105442183 | 105552433 | 110251 |
| 56262 | LRRC8A | NC_000009.12 | 128882112 | 128918042 | 35931 |
| 22944 | KIN | NC_000010.11 | 7750962 | 7788027 | 37066 |
| 1.01E+08 | TIMM23B | NC_000010.11 | 49942033 | 49988221 | 46189 |
| 1E+08 | KLLN | NC_000010.11 | 87859161 | 87863437 | 4277 |
| 85450 | ITPRIP | NC_000010.11 | 104309696 | 104338493 | 28798 |
| 977 | CD151 | NC_000011.10 | 832952 | 838835 | 5884 |
| 89797 | NAV2 | NC_000011.10 | 19345200 | 20121601 | 776402 |
| 931 | MS4A1 | NC_000011.10 | 60455809 | 60470752 | 14944 |
| 220064 | LTO1 | NC_000011.10 | 69665563 | 69675397 | 9835 |
| 7225 | TRPC6 | NC_000011.10 | 101451470 | 101583928 | 132459 |
| 90952 | ESAM | NC_000011.10 | 124753123 | 124762327 | 9205 |
| 259296 | TAS2R50 | NC_000012.12 | 10985913 | 10986912 | 1000 |
| 54407 | SLC38A2 | NC_000012.12 | 46358188 | 46372862 | 14675 |
| 11081 | KERA | NC_000012.12 | 91050491 | 91058354 | 7864 |
| 79794 | C12orf49 | NC_000012.12 | 116713320 | 116738100 | 24781 |
| 55504 | TNFRSF19 | NC_000013.11 | 23570248 | 23676105 | 105858 |
| 729240 | PRR20C | NC_000013.11 | 57154061 | 57157082 | 3022 |
| 54930 | HAUS4 | NC_000014.9 | 22946228 | 22957142 | 10915 |
| 145407 | ARMH4 | NC_000014.9 | 57993536 | 58152305 | 158770 |
| 85439 | STON2 | NC_000014.9 | 81260650 | 81436464 | 175815 |
| 7337 | UBE3A | NC_000015.10 | 25337234 | 25439381 | 102148 |
| 50506 | DUOX2 | NC_000015.10 | 45092648 | 45114161 | 21514 |
| 22801 | ITGA11 | NC_000015.10 | 68297433 | 68432312 | 134880 |
| 8826 | IQGAP1 | NC_000015.10 | 90388241 | 90502243 | 114003 |
| 146562 | C16orf71 | NC_000016.10 | 4734288 | 4749396 | 15109 |
| 23475 | QPRT | NC_000016.10 | 29670588 | 29698699 | 28112 |
| 55336 | FBXL8 | NC_000016.10 | 67159988 | 67164174 | 4187 |
| 54758 | KLHDC4 | NC_000016.10 | 87693537 | 87765992 | 72456 |
| 1949 | EFNB3 | NC_000017.11 | 7705202 | 7711375 | 6174 |
| 399687 | MYO18A | NC_000017.11 | 29073510 | 29180389 | 106880 |
| 9001 | HAP1 | NC_000017.11 | 41722639 | 41734646 | 12008 |
| 3131 | HLF | NC_000017.11 | 55264960 | 55325176 | 60217 |
| 80022 | MYO15B | NC_000017.11 | 75587545 | 75626851 | 39307 |
| 84617 | TUBB6 | NC_000018.10 | 12307669 | 12329826 | 22158 |
| 10892 | MALT1 | NC_000018.10 | 58671386 | 58753806 | 82421 |
| 55620 | STAP2 | NC_000019.10 | 4324043 | 4338877 | 14835 |
| 22983 | MAST1 | NC_000019.10 | 12833931 | 12874953 | 41023 |
| 342865 | VSTM2B | NC_000019.10 | 29525431 | 29564555 | 39125 |
| 11100 | HNRNPUL1 | NC_000019.10 | 41262476 | 41307783 | 45308 |
| 3661 | IRF3 | NC_000019.10 | 49659569 | 49665875 | 6307 |
| 284306 | ZNF547 | NC_000019.10 | 57363435 | 57379559 | 16125 |
| 64412 | GZF1 | NC_000020.11 | 23361585 | 23375399 | 13815 |
| 8202 | NCOA3 | NC_000020.11 | 47501857 | 47656877 | 155021 |
| 337978 | KRTAP21-2 | NC_000021.9 | 30746794 | 30747233 | 440 |
| 51586 | MED15 | NC_000022.11 | 20507542 | 20587632 | 80091 |
| 25829 | TMEM184B | NC_000022.11 | 38219291 | 38273034 | 53744 |
| 171483 | FAM9B | NC_000023.11 | 9024232 | 9034127 | 9896 |
| 2623 | GATA1 | NC_000023.11 | 48786540 | 48794311 | 7772 |
| 27330 | RPS6KA6 | NC_000023.11 | 84058346 | 84187935 | 129590 |
| 10495 | ENOX2 | NC_000023.11 | 130622330 | 130903317 | 280988 |
| 9085 | CDY1 | NC_000024.10 | 25622095 | 25625511 | 3417 |

### Example 4—Protein Analysis of a Patient Sample Using a 100-Plex Protein Panel for Sensor

This example describes the testing of a patient sample of an unbiased 100-protein panel of the human exome.

An exemplary 100-plex protein panel (e.g., Table 7) is designed and antibodies specific to each protein are selected. A sensor plate layout is shown in FIG. 1E. The wells are placed in a SBS-96 format, and each well contains a 10 by 10 grid. Each grid has a nanostructure array. All wells are activated by glutaraldehyde and (3-aminopropyl)-trimethoxysilane (APTMS). Next, antibodies specific to each of the proteins in the 100-plex panel are functionalized on the respective sensor array in each grid using printing technologies. Each 96 plate contains 96 wells, which can run 48 samples in duplicate. Plasma or serum samples from a test group, for example, a group of subjects to be interrogated for protein associations to a phenotype (e.g., a disease group) and control group are added to the wells. Digital and analog signals from each of the sensor arrays are analyzed to cover a large dynamic range of protein concentrations. The protein concentrations from the control and test groups are compared. A set of biomarkers is thus identified to best differentiate the test group from the control group.

### Example 5—Protein Analysis of a Patient Sample Using a 100-Plex Protein Panel for Sensor

This example describes the testing of a patient sample of an unbiased 100-protein panel using a sandwich immunoassay.

An exemplary 100-plex protein panel (e.g., Table 5 or Table 7) is designed and first antibodies specific to each protein are selected. A sensor plate layout is shown in FIG. 1E.

Plasma or serum samples from a test group, for example, a group of subjects to be interrogated for protein associations to a phenotype (e.g., a disease group) and a control group are added to the wells to be analyzed for the presence and/or amount of the target analyte. The sample is added to the well under conditions that permit the first antibody to form a first antibody-analyte complex, if the analyte is present in the sample. Then a second group of antibodies (secondary antibodies) that binds the analyte of interest is added under conditions to permit the second antibody to form a second antibody-analyte complex. The binding of the analyte to the first and second antibody results in a complex in a “sandwich” configuration (FIG. 14E). Digital and analog signals from each of the sensor arrays are analyzed to cover a large dynamic range of protein concentrations. The protein concentrations from the control and test groups are compared. A set of biomarkers is thus identified to best differentiate the test group from the control group.

### Example 6—Design of a Gasket-Approach Protein Panel for Sensor

This example describes an exemplary sensor using the gasket-approach for determination of protein levels.

A gasket approach was used, following the layout depicted in FIG. 15A, in a 96-well plate (“SBS 96”). Each well of the plate was divided into four small wells using a first gasket, and a single antibody was spotted in each small well to create a customized multiplex assay (e.g., 4-small wells/well of 96-well plate, for 384-wells in total). With reference to FIG. 15A antibodies specific to IL-1β, IL-2, IL-6, and IL-8 were deposited into each of the four small wells in locations A1, A3, A5, A7, A9 and A11 of the plate; antibodies specific to IL-10, IL-15, GM-CSF and IP-10 were deposited into each of the four small wells in A2, A4, A6, A8, A10 and A12 of the plate. The same patterns were repeated for rows B to H. The antibody solution in each well was incubated for 2 hours at a concentration of 5 μg/mL. Next, the first gasket layer was peeled off, the chip was dried with nitrogen gas, and stored at 4° C. for further use. Before applying the test sample, a second gasket layer covering two neighboring SBS 96 single wells (e.g., A1 and A2, thus, eight small wells from the first gasket layer) was applied to the chip.

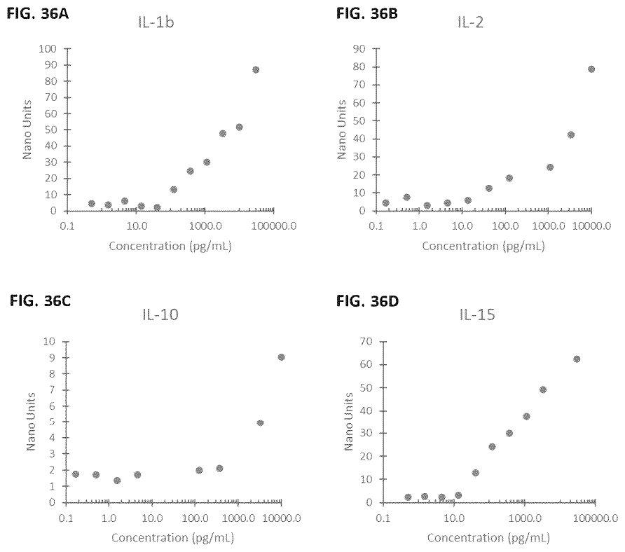

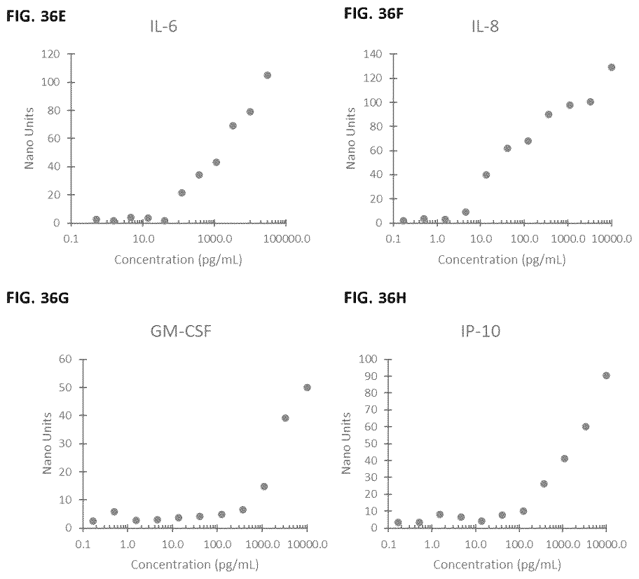

A mixture of recombinant proteins of IL-1β, IL-2, IL-6, IL-8, IL-10, IL-15, GM-CSF and IP-10, each at a concentration of 10 ng/mL, was spiked into the buffer, diluted in 3× series for a total of 12-dilution points, and applied to different wells of the second gasket. After 2 hours of incubation, a cocktail solution of biotinylated detection antibodies to IL-1β, IL-2, IL-6, IL-8, IL-10, IL-15, GM-CSF and IP-10 at 0.5 μg/mL concentration was applied to each well of the second gasket. After regular washing steps, 0.5 μg/ml streptavidin-HRP solution was applied to each well of the second gasket and incubated for 0.5 hours. Then, a reformulated TMB (3, 3′, 5, 5′-tetramethylbenzidine) solution was applied to each well of the second gasket to produce a non-soluble sediment on the nanoneedle sensors in each well. A dark field imaging instrument was used to capture all images of the nanoneedles. The needles that displayed color changes were counted and proportion to the total number of nanoneedles was used to determine the percentage shown as “Nano Unit” in FIG. 36. FIGS. 36A-36H are graphs showing the detection of IL-1b (FIG. 36A), IL-2 (FIG. 36B), IL-10 (FIG. 36C), IL-15 (FIG. 36D), IL-6 (FIG. 36E), IL-8 (FIG. 36F), GM-CSF (FIG. 36G), and IP-10 (FIG. 36H). As a whole, FIG. 36 shows that the number of nanoneedles that have the color change output increases as the concentrations of the proteins increase.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of determining a protein panel comprising a set of test proteins selected from a whole protein coding genome of a species to which a study subject belongs or is related to, the method comprising the steps of:
(a) splicing protein coding genes from a whole genome to construct a protein-coding genome;
(b) determining a plurality of marker locations substantially evenly spaced across the protein-coding genome; and
(c) identifying a protein associated with each marker location across the protein-coding genome to produce the set of test proteins, wherein each protein is encoded by a gene that includes a single nucleotide polymorphism (SNP) located close to each marker location in the protein-coding genome.

2. The method of claim 1, wherein the protein coding genes are exons, and the protein-coding genome is an exome.

3. The method of claim 1, wherein the protein coding genes are coding sequence (CDS) regions and the protein-coding genome is an exome-CDS.

4. The method of claim 1, wherein the SNPS are synonymous SNPS, non-synonymous SNPS, or a combination thereof.

5. (canceled)

6. The method of claim 1, wherein the SNP is the closest SNP to the marker location in the protein-coding genome, exome or exome-CDS.

7-8. (canceled)

9. A method of determining a protein panel comprising a set of test proteins selected from a whole protein coding genome of a species to which a study subject belongs or is related to, the method comprising the steps of:
(a) splicing protein coding genes from a whole genome to construct a protein-coding genome;
(b) determining a plurality of marker locations substantially evenly spaced across the protein-coding genome; and
(c) identifying a protein associated with each marker location across the protein-coding genome to produce the set of test proteins, wherein each protein is the protein encoded by a region of the protein coding genome in which the associated marker is located.

10. The method of claim 9, wherein the protein coding genes are exons, and the protein-coding genome is an exome.

11. The method of claim 9, wherein the protein coding genes are coding sequence (CDS) regions and the protein-coding genome is an exome-CDS.

12. (canceled)

13. A sensor for detecting presence, or quantifying the amount of a plurality of proteins in a sample harvested from a study subject thereby to conduct a bias-free proteome, exome or exome-CDS association study on the sample, the sensor comprising:
a plate defining a plurality of addressable wells, each well comprising a grid disposed therein, wherein (i) the grid comprises a plurality of nanostructure arrays with each nanostructure array comprising a plurality of nanostructures, (ii) each nanostructure array is functionalized with one or more binding moieties for binding one or more proteins of a set of test proteins for conducting a bias-free proteome, exome or exome-CDS association study, and (iii) each nanostructure is integral with at least one of a planar support or a flexible substrate.

14. The sensor of claim 46, wherein the SNPs are synonymous SNPs, non-synonymous SNPs, or a combination thereof.

15-16. (canceled)

17. The sensor of claim 46, wherein the SNP is the closest ASNP to the marker location in the protein-coding genome, exome or exome-CDS.

18-19. (canceled)

20. The sensor of claim 46, wherein the SNP is located less than 1,000 bases from a corresponding marker location.

21-23. (canceled)

24. The sensor of claim 13, wherein the binding moiety is an antibody, a nanobody, an aptamer, or an affinity probe.

25-26. (canceled)

27. A method of producing a sensor for detecting the presence, or quantifying the amount, of a plurality of proteins in a sample harvested from a study subject thereby to conduct a bias-free proteome, exome or exome-CDS association study on the sample, the method comprising the steps of:
(a) determining a plurality of marker locations substantially evenly spaced across an protein-coding genome, exome or exome-CDS of a species to which the study subject belongs or is related to;
(b) identifying a protein associated with each marker location across the protein-coding genome, exome or exome-CDS to produce a set of test proteins, wherein each protein is encoded by a gene that includes a single nucleotide polymorphism (SNP) located closely to each marker location in the exome; and
(c) functionalizing nanostructures of the sensor with a plurality of different binding moieties each capable of binding a protein in the set of test proteins thereby to detect the presence, or quantify the amount, of the test proteins if present in the sample, wherein each nanostructure is integral with at least one of a planar support or a flexible substrate.

28. The method of claim 27, comprising repeating steps (a)-(c) thereby to produce a series of sensors, wherein the marker locations used to create a second sensor are shifted by a predetermined distance from the marker locations used to create a first sensor.

29. The method of claim 27, wherein the SNPs are synonymous SNPs, non-synonymous SNPs, or a combination thereof.

30-31. (canceled)

32. The method of claim 27, wherein the SNP is the closest SNP to the marker location in the protein-coding genome, exome, or exome-CDS.

33-34. (canceled)

35. The method of claim 27, wherein the SNP is located less than 1,000 bases from a corresponding marker location.

36. (canceled)

37. The method of claim 27, wherein the binding moiety is an antibody, nanobody, aptamer or an affinity probe.

38. A sensor produced by the method of claim 27.

39. (canceled)

40. A method of conducting a bias-free proteome, exome or exome-CDS wide association study on a sample of interest, the method comprising:
(a) applying a portion of the sample to a sensor of claim 13;
(b) detecting detectable signals from the nanostructures of the sensor; and
(c) determining from the detectable signals the presence and/or amount of the test proteins in the sample.

41. The method of claim 40, further comprising repeating steps (a)-(c) with at least one additional sensor to screen a protein panel of the sample of interest.

42. The method of claim 40, wherein detecting detectable signals comprises detecting a change in a property of at least a portion of the nanostructures.

43. (canceled)

44. The method of claim 40, wherein the sample is not diluted prior to application to the sensor.

45. (canceled)

46. The sensor of claim 13, wherein the set of test proteins has previously been determined by:
(a) determining a plurality of marker locations substantially evenly spaced across a protein-coding genome, exome or exome-CDS of a species to which the study subject belongs or is related to; and
(b) identifying a protein associated with each marker location across the protein-coding genome, exome or exome-CDS to produce the set of test proteins, wherein each protein is encoded by a gene that includes a single nucleotide polymorphism (SNP) located close to each marker location in the exome.
